(19) United States
(12) Patent Application Publication
(10) Pub. No.: US 2023/0130766 A1
(43) Pub. Date: Apr. 27, 2023

(54) MONO AND COMBINATION THERAPIES WITH ULK1/2 INHIBITORS

(71) Applicants: SALK INSTITUTE FOR BIOLOGICAL STUDIES, La Jolla, CA (US); SANFORD BURNHAM PREBYS MEDICAL DISCOVERY INSTITUTE, La Jolla, CA (US)

(72) Inventors: Nicholas D.P. COSFORD, La Jolla, CA (US); Reuben J. SHAW, La Jolla, CA (US); Nicole A. BAKAS, La Jolla, CA (US); Allison S. LIMPERT, La Jolla, CA (US); Sonja N. BRUN, La Jolla, CA (US); Mitchell VAMOS, La Jolla, CA (US)

(21) Appl. No.: 17/799,641

(22) PCT Filed: Feb. 12, 2021
(86) PCT No.: PCT/US21/18044

§ 371 Date: Aug. 12, 2022

Related U.S. Application Data
(60) Provisional application No. 62/977,041, filed on Feb. 14, 2020.

(51) Int. Cl. A61K 31/506; A61P 35/00; A61K 45/06

(52) U.S. Cl. CPC A61K 31/506; A61P 35/00; A61K 45/06

(57) ABSTRACT

Provided herein are methods of treating diseases, including cancer, with ULK inhibitors, both as monotherapies and in combination with other therapeutic agents.

### CROSS-REFERENCE TO RELATED APPLICATIONS

This application claims priority to U.S. Provisional Application No. 62/977,041 filed Feb. 14, 2020, entitled “Mono and Combination Therapies with ULK1/2 Inhibitors,” the disclosure of which is hereby incorporated by reference in its entirety.

### STATEMENT AS TO FEDERALLY SPONSORED RESEARCH

This invention was made with government support under T32 grant number 1T32CA211036 awarded by NIH/NCI. The government has certain rights in the invention.

### BACKGROUND

Autophagy is a central cellular mechanism for the elimination of damaged proteins, protein complexes, and organelles. This conserved process plays crucial roles in the cellular response to nutrient deprivation and other stresses, in addition to being required for proper cellular and tissue homeostasis during embryonic development and in defense against pathogens. Defects in autophagy pathways are associated with certain human pathologies, including infectious diseases, neurodegenerative disorders, and cancer. In spite of these highly conserved fundamental cellular functions, the molecular and biochemical details of how autophagy is initiated for different cargoes, and the coordination of steps starting from autophagosome initiation to ultimate fusion with the lysosome remain poorly understood.

### SUMMARY

Provided herein are inhibitors of unc-51 like autophagy activating kinase (ULK) proteins. In some embodiments, the inhibitors inhibit ULK1. In some embodiments, the inhibitors are specific for ULK1. In some embodiments, the inhibitors inhibit both ULK1 and ULK2. In some instances, the inhibitors provided herein are useful for the treatment of various diseases, including cancer.

In many instances, ULK1 and ULK2 are important proteins that regulate autophagy in mammalian cells. In certain instances, ULK1 and ULK2 are activated under conditions of nutrient deprivation by several upstream signals, which is followed by the initiation of autophagy. The requirement for ULK1 and ULK2 in autophagy initiation has been studied in the context of nutrient deprivation. While ULK1 appears to be the most essential for autophagy, in some instances, ULK1 and ULK2 show high functional redundancy. The kinase domains of ULK1 and ULK2 share 78% sequence homology, suggesting ULK2 may compensate for the loss of ULK1 in some instances. In some instances, nutrient dependent autophagy may only be eliminated if both ULK1 and ULK2 are inhibited. In some instances, inhibition of ULK1 alone is sufficient, e.g. for providing a therapeutic benefit, such as in any method provided herein, for normalizing autophagy in a cancer cell, or other beneficial result. In other instances, inhibition of ULK1 and ULK2 results in a therapeutic benefit, such as tumor shrinkage, tumor cell death, or slowed rate of tumor growth.

In some embodiments, the compounds provided herein are inhibitors of ULK. In some embodiments, the compounds inhibit ULK1. In some embodiments, the compounds are specific for ULK1. In some embodiments, the compounds inhibit both ULK1 and ULK2. In some embodiments, the diseases provided herein are treatable with an inhibitor specific for ULK1. In some instances, ULK2 may compensate for loss of ULK1 function. In some embodiments, the diseases provided herein require treatment with a compound that inhibits both ULK1 and ULK2.

Provided herein are ULK inhibitors which are useful in the treatment of diseases. Also provided herein are methods of using ULK inhibitors as a monotherapy for the treatment of diseases, including cancer. In some aspects, the ULK inhibitors are useful for the treatment of diseases, including cancer, in combination with other therapeutic agents, including therapeutic agents that make up current standard of care therapies. In some embodiments, the compound is specific for ULK1. In some embodiments, the compound inhibits both ULK1 and ULK2.

In one aspect, provided herein, is a method for treating cancer in a subject in need thereof, the method comprising: administering to the subject a therapeutically effective amount of a ULK inhibitor having a structure of Formula A:

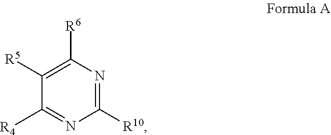

wherein in Formula A:

R¹⁰ is halogen; —OR¹¹, wherein R¹¹ is selected from the group consisting of H, optionally substituted aryl and optionally substituted heteroaryl; —NR¹R², wherein R¹ is H, —C(═O)R¹², where R¹² is C₁-C₆ alkyl or C₁-C₆ cycloalkyl, or optionally substituted alkyl and R² is H, optionally substituted aryl, optionally substituted heteroaryl, optionally substituted cycloalkyl, or optionally substituted alkyl, or NR¹R² together form a heterocycle;

R⁴ is optionally substituted amino, optionally substituted aryloxy, optionally substituted heteroaryloxy, optionally substituted alkoxy, N-heterocyclic, optionally substituted thiol, optionally substituted alkyl, hydroxyl or halogen; or R⁴ and R¹⁰ together form a cyclic structure;

R⁵ is H, hydroxyl, optionally substituted alkyl (e.g., fluoroalkyl), halogen, optionally substituted alkoxy, or optionally substituted aryl, optionally substituted carboxyl, cyano, or nitro; or R⁵ and R⁶ together form a cyclic structure; and

R⁶ is H, halogen, or haloalkyl.

In another aspect, provided herein, is a method for treating cancer in a subject in need thereof, the method comprising administering to the subject a therapeutically effective amount of a ULK inhibitor. In some embodiments, the ULK inhibitor is specific for ULK1. In some embodiments, the ULK inhibitor inhibits ULK1 and ULK2.

In some embodiments, the ULK inhibitor is administered as a monotherapy. In some embodiments, the subject is not simultaneously administered a mechanistic target of rapamycin (mTOR) inhibitor.

In some embodiments, the method further comprises administering to the subject an additional therapeutic agent. In some embodiments, the additional therapeutic agent is a standard of care therapy. In some embodiments, the additional therapeutic agent is not an mTOR inhibitor. In some embodiments, the additional therapeutic agent comprises carboplatin. In some embodiments, the additional therapeutic agent comprises a carboplatin analog. In some embodiments, the additional therapeutic agent comprises a mitogen-activated protein kinase (MEK) inhibitor. In some embodiments, the additional therapeutic agent comprises trametinib. In some embodiments, the additional therapeutic agent is gemcitabine. In some embodiments, the additional therapeutic agent is a nucleoside analog. In some embodiments, the additional therapeutic agent is a poly (ADP-ribose) polymerase (PARP) inhibitor. In some embodiments, the additional therapeutic agent is olaparib.

In some embodiments, the cancer is lung cancer, breast cancer, or pancreatic cancer. In some embodiments, the cancer is lung cancer. In some embodiments, the cancer is non-small cell lung cancer (NSCLC). In some embodiments, the cancer is pancreatic cancer. In some embodiments, the pancreatic cancer is pancreatic ductal adenocarcinoma (PDAC). In some embodiments, the cancer is breast cancer. In some embodiments, the cancer is breast cancer. In some embodiments, the breast cancer is triple-negative breast cancer (TNBC).

In some embodiments, the cancer is refractory to a prior treatment. In some embodiments, the cancer is refractory to carboplatin or a carboplatin analog. In some embodiments, the cancer is refractory to an MEK inhibitor. In some embodiments, the cancer is refractory to trametinib, cobimetinib, binimetinib, or selumetinib. In some embodiments, the cancer is refractory to erlotinib, gefitinib, osimertinib, crizotinib, pemetrexed, docetaxol, or pembroluzimab. In some embodiments, the cancer is refractory to a PARP inhibitor. In some embodiments, the cancer is refractory to FOLFIRINOX (5-fluorouracil, leucovorin, irinotecan, and oxaliplatin), gemcitabine, gemcitabine/abraxane, everolimus, erlotinib, or sunitinib. In some embodiments, the cancer is refractory to anastrozole, exemestane, letrozole, or tamoxifen.

In some embodiments, the subject was treated with at least one additional therapeutic agent prior to administration of the ULK inhibitor. In some embodiments, the compound and the at least one additional therapeutic agent are administered concomitantly. In some embodiments, the ULK inhibitor and the at least one additional therapeutic agent are administered concomitantly at the start of treatment with the ULK inhibitor. In some embodiments, the prior treatment is ceased prior to administration of the ULK inhibitor.

In some embodiments, treatment with the additional therapeutic agent induces a cytostatic response. In some embodiments, treatment with the additional therapeutic agent induces a cytostatic response in disease tissue. In some embodiments, treatment with the additional therapeutic agent without a ULK inhibitor induces a cytostatic response. In some embodiments, administering a ULK inhibitor enhances the efficacy of the additional therapeutic agent. In some embodiments, administering a ULK inhibitor enhances the efficacy of the additional therapeutic agent by at least 10%, at least 20%, at least 30%, at least 40%, at least 50%, at least 60%, at least 70%, at least 80%, at least 90%, or at least 95%. In some embodiments, efficacy is measured by reduction in tumor volume, slowed tumor growth, or inhibition of tumor growth.

In another aspect, provided herein, is a method for treating refractory cancer in a subject in need thereof, the method comprising administering to the subject a ULK inhibitor. In some embodiments, the cancer is refractory to a standard of care therapy. In some embodiments, the refractory cancer is lung cancer. In some embodiments, the refractory cancer is non-small cell lung cancer. In some embodiments, the cancer is refractory to treatment with carboplatin. In some embodiments, the method further comprises co-administering to the subject carboplatin in combination with the ULK inhibitor.

In some embodiments, the refractory cancer is pancreatic cancer. In some embodiments, the cancer is refractory to an MEK inhibitor. In some embodiments, the cancer is refractory to trametinib. In some embodiments, the method further comprises co-administering to the subject trametinib in combination with the ULK inhibitor.

In some embodiments, the disease or disorder is characterized by abnormal autophagy. In some embodiments, the abnormal autophagy is therapeutically induced. In some embodiments, the disease or disorder is refractory. In some embodiments, the disease or disorder is refractory to treatment with an additional therapeutic agent. In some embodiments, the disease or disorder is resistant to treatment with an additional therapeutic agent.

In one aspect, provided herein, is a method for treating a disease in a subject in need thereof, the method comprising administering to the subject a therapeutically effective amount of a ULK inhibitor. In some embodiments, the disease is characterized by abnormal autophagy. In some embodiments, the disease is Tuberous Sclerosis Complex (TSC). In some embodiments, the disease is lymphangioleiomyomatosis (LAM). In some embodiments, the subject is not administered an mTOR inhibitor.

In some embodiments, the ULK inhibitor has a structure of Formula I:

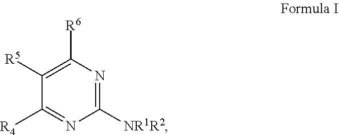

wherein in Formula I:

R¹ and R² are each individually selected from the group consisting of H, optionally substituted aryl, optionally substituted heteroaryl, optionally substituted cycloalkyl, and optionally substituted alkyl, or NR¹R² together form a heterocycle;

R⁴ is selected from the group consisting of optionally substituted amino, optionally substituted aryloxy, optionally substituted heteroaryloxy, optionally substituted alkoxy, N-heterocyclic, optionally substituted thiol, and optionally substituted alkyl;

R⁵ is selected from the group consisting of H, hydroxyl, optionally substituted alkyl (e.g., fluoroalkyl such as trifluoromethyl), halogen, optionally substituted alkoxy, and optionally substituted aryl; and

R⁶ is H.

In some embodiments, the ULK inhibitor is selected from the group consisting of a 2-(substituted)amino-4-(substituted)amino-5-halo-pyrimidine, 2-(substituted)amino-4-(substituted)amino-5-(halo)alkyl-pyrimidine, 2-(substituted)amino-4-(substituted)oxo-5-halo-pyrimidine, 2-(substituted)amino-4-(substituted)oxo-5-(halo)alkyl-pyrimidine, 2-(substituted)amino-4-(substituted)thio-5-halo-pyrimidine, and 2-(substituted)amino-4-(substituted)thio-5-(halo)alkyl-pyrimidine.

In some embodiments, the method comprises inhibiting ULK. In some embodiments, the method further comprises decreasing phosphorylation of autophagy-related protein 13 (ATG13) in the subject. In some embodiments, the method further comprises decreasing the amount of ATG13 in the subject. In some embodiments, the method further comprises degrading ATG13 in diseased tissue of the subject.

In some embodiments, the subject comprises a mutation in at least one of KRAS, PTEN, TSC1, TSC2, PIk3CA, P53, STK11 (a.k.a. LKB1), KEAP1, NRF2, or EGFR. In some embodiments, the subject comprises a mutation in at least one of KRAS, PTEN, TSC1, TSC2, PIk3CA, P53, STK11 (a.k.a. LKB1), KEAP1, NRF2, ALK4, GNAS or EGFR. In some embodiments, the subject comprises a mutation in at least one of SMAD4, p16/CDKM2A, or BRCA2. In some embodiments, the subject comprises a mutation in at least one of KRAS, PTEN, TSC1, TSC2, PIk3CA, P53, STK11 (a.k.a. LKB1), KEAP1, NRF2, EGFR, SMAD4, p16/CDKM2A, BRCA2, ALK4, or GNAS.

In some embodiments, the compound is administered as a pharmaceutical composition comprising the compound and a pharmaceutically acceptable carrier. In some embodiments, the pharmaceutical composition is a liquid formulation. In some embodiments, the pharmaceutical composition is formulated for intravenous or intraperitoneal administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows Western blot analysis of clustered regularly interspaced short palindromic repeat (CRISPR) knockout H460 polyclonal cell lines.

FIG. 1B shows Western blot analysis of CRISPR knockout A549 polyclonal cell lines.

FIG. 2A shows differences in H460 tumor growth in tumors containing genes knocked out with CRISPR.

FIG. 2B shows differences in A549 tumor growth in tumors containing genes knocked out with CRISPR.

FIG. 3A shows tumor volume in H358 xenografts in mice treated with vehicle or a ULK inhibitor over time.

FIG. 3B shows percent tumor growth in H358 xenografts in mice treated with vehicle or a ULK inhibitor over time.

FIG. 3C shows tumor mass in H358 xenografts in mice treated with vehicle or a ULK inhibitor after 35 days.

FIG. 4A shows tumor volume in H460 xenografts in mice treated with vehicle or a ULK inhibitor over time.

FIG. 4B shows percent tumor growth in H460 xenografts in mice treated with vehicle or a ULK inhibitor over time.

FIG. 4C shows tumor mass in H460 xenografts in mice treated with vehicle or a ULK inhibitor after 9 days.

FIG. 5A shows tumor volume in A549 xenografts in mice treated with vehicle, a ULK inhibitor, carboplatin, or a combination thereof over time.

FIG. 5B shows tumor mass in A549 xenografts in mice treated with vehicle, a ULK inhibitor, carboplatin, or a combination thereof after 42 days.

FIG. 6 shows a Western blot of A549 xenograft tumors following treatment with vehicle or a ULK inhibitor.

FIG. 7A shows tumor volume in A549 xenografts in mice treated with vehicle, a ULK inhibitor, or carboplatin over time.

FIG. 7B shows tumor mass in A549 xenografts in mice treated with vehicle, a ULK inhibitor, or carboplatin after 32 days.

FIG. 8 shows Western blot analysis of CRISPR knockout MIA PaCa-2 polyclonal cell lines.

FIG. 9A shows differences in MIA PaCa-2 tumor growth in tumors containing genes knocked out with CRISPR over time.

FIG. 9B shows differences in MIA PaCa-2 tumor growth in tumors containing genes knocked out with CRISPR after 49 days.

FIG. 10 shows tumor volume in MIA PaCa-2 xenografts in mice treated with vehicle, a ULK inhibitor, or gemcitabine over time.

FIG. 11 shows a Western blot of MIA PaCa-2 xenograft tumors following treatment with vehicle or a ULK inhibitor.

FIG. 12A shows tumor volume in MIA PaCa-2 xenografts in mice treated with vehicle, a ULK inhibitor, trametinib, or a combination thereof over time.

FIG. 12B shows percent tumor growth in MIA PaCa-2 xenografts in mice treated with vehicle, a ULK inhibitor, trametinib, or a combination thereof over time.

FIG. 12C shows tumor mass MIA PaCa-2 xenografts in mice treated with vehicle, a ULK inhibitor, trametinib, or a combination thereof after 25 days.

FIG. 13A is a matrix showing the combined effects of the concentration of Compound 349 (horizontal direction) and the concentration of olaparib (vertical direction) on the inhibition of ULK1/2 and PARP. Darker shading indicates synergy, whereas lighter shading indicates antagonism.

FIG. 13B is a bar graph showing percent cell viability after exposure to the indicated compounds, or combinations thereof.

FIG. 14 is a graph showing the percentage of cells exhibiting high, intermediate, and low autophagic flux as a function of exposure to the indicated compounds, or combinations thereof.

### DETAILED DESCRIPTION

Provided herein are methods of treating a disease with a ULK inhibitor as a monotherapy. Also provided herein are methods of treating a disease with a ULK inhibitor and an additional therapeutic agent. Further provided herein are compounds useful as ULK inhibitors. In some instances, the ULK inhibitor is a ULK1 specific inhibitor. In some instances, the ULK inhibitor inhibits both ULK1 and ULK2.

Autophagy

In certain instances, autophagy is a cellular response to loss of nutrients in which cells catabolize various proteins and organelles to provide building blocks and critical metabolites needed for cell survival. In some instances, autophagy plays an important homeostatic role in many tissues by removing protein aggregates and defective organelles that accumulate with cellular damage over time. While genetics first defined the core components of autophagy conserved across all eukaryotes, the molecular details of how the different autophagy complexes regulate one another and the precise temporal and spatial ordering of biochemical events involved in autophagy induction are typically considered to be poorly understood currently.

In healthy individuals, normal autophagy is, in certain instances, an important process for balancing sources of energy at critical times in development and in response to nutrient stress. In certain instances, autophagy also plays a housekeeping role in removing misfolded or aggregated proteins, clearing damaged organelles, such as mitochondria, endoplasmic reticulum and peroxisomes, as well as eliminating intracellular pathogens. Thus, autophagy is often thought of as a survival mechanism. In various instances, autophagy is either non-selective or selective in the removal of specific organelles, ribosomes and protein aggregates. In addition to elimination of intracellular aggregates and damaged organelles, in certain instances, autophagy promotes cellular senescence and cell surface antigen presentation, protects against genome instability and prevents or inhibits necrosis, giving it an important role in preventing, treating, or inhibiting diseases such as cancer, neurodegeneration, cardiomyopathy, diabetes, liver disease, autoimmune diseases and infections.

In some instances, defects in autophagy pathways are associated with a number of human pathologies, including infectious diseases, neurodegenerative disorders, and cancer. In some instances, the role of autophagy differs in different stages of cancer development; for example, in some instances, initially, autophagy has a preventive effect against cancer, but once a tumor develops, the cancer cells, in certain instances, utilize autophagy for their own cytoprotection. In some cancers, the mutations that cause uncontrolled cell growth which results in the formation of tumors or other cancerous tissue also effectuates changes in autophagy. In some instances, these changes in the autophagic pathways in the cancer cells results in increased survivability and durability of cancer cells. In some instances, this leads to the cells resisting apoptosis and cell death in response to standard cancer treatments, thus reducing the efficacy of cancer therapeutics. In certain instances, rather than killing the cancer cells, the therapeutics merely have the effect of arresting cancer tissue growth, with the cancer tissue entering a cystostatic phase upon treatment. Consequently, in some instances, the cancerous tissue is not killed during treatment, the growth is simply arrested. Upon cessation of treatment, the cancerous tissue is able to resume growth, thus increasing symptoms and complications for the patient. In light of this, in some instances, the addition of a therapeutic that disrupts autophagy has the effect of converting the cytostatic response of the cancer cells to cancer cell death.

In certain cancers, the changes in autophagy caused by the cancer are important for the survival of the cancer cells. As the mutations that cause cancer result in uncontrolled cell growth, in some instances, these cells rely on autophagy to properly regulate the consumption of nutrients to ensure the survival of the cells in conditions that would cause the death of a healthy cell. Thus, methods of inhibiting autophagy in cells present, in certain instances, a method of treating cancer without the need of an additional cancer therapeutic.

ULK1 and ULK2

In many instances, ULK1 and/or ULK2 are important proteins in regulating autophagy in mammalian cells. In certain instances, ULK1 and/or ULK2 are activated under conditions of nutrient deprivation by several upstream signals, which is followed by the initiation of autophagy. The requirement for ULK1 and/or ULK2 in autophagy initiation has been studied in the context of nutrient deprivation.

In certain instances, the ULK1 complex, combining ULK1, ATG (autophagy-related protein) 13 (ATG13), FIP200 (focal adhesion kinase family interacting protein of 200 kDa), and ATG101 is one of the first protein complexes that comes in to play in the initiation and formation of autophagosomes when an autophagic response is initiated. Additionally, ULK1 is considered to be unique as a core conserved component of the autophagy pathway because it is a serine/threonine kinase, making it a particularly unique target of opportunity for development of compounds to control autophagy. Equally importantly for a clinical therapeutic index for agents inhibiting ULK1, mice genetically engineered to completely lack ULK1 are viable without significant pathology. Thus, in many instances, a ULK1 selective kinase inhibitor is well tolerated by normal tissues, but not by tumor cells that have become reliant on ULK1 mediated autophagy for survival.

In some instances, ULK2 takes over the functional role of ULK1 when ULK1 function has been inhibited. Thus, in some cases, an inhibitor that is effective for both ULK1 and ULK2 is desirable to mitigate this effect.

### I. METHODS OF TREATMENT

In some instances, ULK inhibitors are used and/or useful in the treatment of cancer and/or ULK mediated disorders. Surprisingly, in certain instances, ULK inhibitors are efficacious as a monotherapy. In other instances, it is also surprising that ULK inhibitors are used/useful in augmenting or improving standard of care therapies. In some instances, the standard of care therapies do not involve mTOR inhibitors. In some instances, the cancer and ULK mediated disorders do not implicate mTOR. In some instances, the ULK inhibitor inhibits ULK1. In some instances, the ULK inhibitor is a ULK1 specific inhibitor. In some instances, the ULK inhibitor inhibits both ULK1 and ULK2.

Monotherapy

In one aspect, provided herein, is a method of treating a disease or disorder with a ULK inhibitor. In various embodiments, the ULK inhibitor is administered alone to treat a disease or disorder. In some embodiments, the method comprises administering to a subject in need thereof a therapeutically effective amount of a ULK inhibitor.

In some embodiments, the ULK inhibitor is administered as a monotherapy. In some embodiments, the ULK inhibitor is the sole therapeutic agent administered to the patient for the treatment of the disease or disorder. In some embodiments, the ULK inhibitor is the sole anti-cancer agent administered to the patient. In some embodiments, the ULK inhibitor is administered as a monotherapy with additional inactive ingredients as part of a pharmaceutical formulation. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, the disease or disorder is characterized by abnormal autophagy. In some embodiments, the abnormal autophagy is therapeutically induced. In some embodiments, the disease or disorder is refractory. In some embodiments, the disease or disorder is refractory to treatment with an additional therapeutic agent. In some embodiments, the disease or disorder is resistant to treatment with an additional therapeutic agent. In some embodiments, the additional therapeutic agent is a standard of care therapy.

In some embodiments, the disease or disorder treated with a ULK inhibitor as a monotherapy is cancer. In some embodiments, the cancer is lung cancer, breast cancer, or pancreatic cancer. In some embodiments, the cancer is refractory. In some embodiments, the cancer is refractory to a standard of care therapy.

In some embodiments, the cancer is lung cancer. In specific embodiments, the lung cancer is non-small cell lung cancer. In some embodiments, the cancer is an advanced stage non-small cell lung cancer. In some embodiments, the cancer comprises a tumor. In some embodiments, the non-small cell lung cancer comprises a tumor. In some embodiments, the non-small cell lung cancer is characterized by abnormal autophagy. In some embodiments, the lung cancer is refractory. In some embodiments, the lung cancer is refractory to treatment with carboplatin. In some embodiments, the non-small cell lung cancer is refractory. In some embodiments, the non-small cell lung cancer is refractory to treatment with carboplatin. In some embodiments, the lung cancer is refractory to treatment with erlotinib, gefitinib, osimertinib, or crizotinib. In some embodiments, the lung cancer is refractory to treatment with pemetrexed, docetaxol, or pembroluzimab. In some embodiments, the lung cancer is refractory to erlotinib, gefitinib, osimertinib, crizotinib, pemetrexed, docetaxol, or pembroluzimab. In some embodiments, the non-small cell lung cancer is refractory to treatment with erlotinib, gefitinib, osimertinib, or crizotinib. In some embodiments, the non-small cell lung cancer is refractory to treatment with pemetrexed, docetaxol, or pembroluzimab. In some embodiments, the non-small cell lung cancer is refractory to erlotinib, gefitinib, osimertinib, crizotinib, pemetrexed, docetaxol, or pembroluzimab. In some embodiments, the lung cancer is refractory to gemcitabine, bortexomib, trastuzumab, vinorelbine, doxorubicin, irinotecan, temsirolimus, sunitinib, nivolumab, or bevacizumab. In some embodiments, the lung cancer is refractory to carboplatin/gemcitabine, carboplatin/paclitaxel/cetuximua, cisplatin/pemetrexed, cisplatin/docetaxel, cisplatin/docetaxel/bevacizumab, everolimus/nab-paclitaxel, or tremelimumab/durvalumab. In some embodiments, the non-small cell lung cancer is refractory to gemcitabine, bortexomib, trastuzumab, vinorelbine, doxorubicin, irinotecan, temsirolimus, sunitinib, nivolumab, or bevacizumab. In some embodiments, the non-small cell lung cancer is refractory to carboplatin/gemcitabine, carboplatin/paclitaxel/cetuximua, cisplatin/pemetrexed, cisplatin/docetaxel, cisplatin/docetaxel/bevacizumab, everolimus/nab-paclitaxel, or tremelimumab/durvalumab. In some embodiments, the subject with lung cancer comprises a mutation in KRAS, PTEN, TSC1, TSC2, PIk3CA, P53, STK11 (a.k.a. LKB1), KEAP1, NRF2, ALK4, GNAS or EGFR.

In some embodiments, the cancer is breast cancer. In some embodiments, the breast cancer comprises a tumor. In some embodiments, the breast cancer is characterized by abnormal autophagy. In some embodiments, the breast cancer is refractory. In some embodiments, the breast cancer is refractory to anastrozole, exemestane, letrozole, or tamoxifen. In some embodiments, the breast cancer is refractory to PARP inhibitor. In some embodiments, the breast cancer is refractory to anastrozole, exemestane, letrozole, tamoxifen, or a PARP inhibitor. In some embodiments, the PARP inhibitor is olaparib, rucaparib, niraparib, or talazoparib. In some embodiments, the breast cancer is refractory to olaparib, rucaparib, niraparib, or talazoparib. In some embodiments, the breast cancer is TNBC.

In some embodiments, the cancer is pancreatic cancer. In some embodiments, the pancreatic cancer comprises a tumor. In some embodiments, the pancreatic cancer is characterized by abnormal autophagy. In some embodiments, the pancreatic cancer is refractory. In some embodiments, the pancreatic cancer is refractory to FOLFIRINOX, gemcitabine, or gemcitabine/abraxane. In some embodiments, the pancreatic cancer is refractory. In some embodiments, the pancreatic cancer is refractory to FOLFIRINOX, gemcitabine, gemcitabine/abraxane, everolimus, erlotinib, or sunitinib. In some embodiments, the pancreatic cancer is refractory to gemcitabine. In some embodiments, the pancreatic cancer is refractory to capeditabine, leucovorin, nab-paclitaxel, nanoliposomal irinotecan, gemcitabine/nab-paclitaxel, pembrolizumab, or cisplatin. In some embodiments, the pancreatic cancer is PDAC. In some embodiments, the subject with pancreatic cancer comprises a mutation in at least one of SMAD4, p16/CDKM2A, or BRCA2.

In some embodiments, the disease or disorder treated with a ULK inhibitor as a monotherapy is LAM. In some embodiments, the disease or disorder treated with a ULK inhibitor as a monotherapy is TSC.

In some embodiments, administering a ULK inhibitor slows progression of the disease or disorder. In some embodiments, administering a ULK inhibitor slows progression of the disease or disorder by at least 10%, at least 20%, at least 30%, at least 40%, at least 50%, at least 60%, at least 70%, at least 80%, at least 90%, or at least 95%. In some embodiments, progression is measured by tumor growth. In some embodiments, administering a ULK inhibitor arrests cancer cell growth. In some embodiments, administering a ULK inhibitor reduces tumor volume. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, the method of treatment comprises decreasing phosphorylation of ATG13 in the subject. In some embodiments, the method comprises degrading ATG13 in diseased tissue of the subject.

In some embodiments, the subject comprises a mutation in KRAS, PTEN, TSC1, TSC2, PIk3CA, P53, STK11 (a.k.a. LKB1), KEAP1, NRF2, or EGFR. In some embodiments, the subject comprises a mutation in KRAS, PTEN, TSC1, TSC2, PIk3CA, P53, STK11 (a.k.a. LKB1), KEAP1, NRF2, ALK4, GNAS or EGFR. In some embodiments, the subject comprises a mutation in at least one of SMAD4, p16/CDKM2A, or BRCA2. In some embodiments, the subject comprises a mutation in at least one of ALK4 or GNAS. In some embodiments, the subject comprises a mutation in at least one of KRAS, PTEN, TSC1, TSC2, PIk3CA, P53, STK11 (a.k.a. LKB1), KEAP1, NRF2, EGFR, SMAD4, p16/CDKM2A, BRCA2, ALK4, or GNAS.

Combination Therapy

Also described herein are combination therapies. In some instances, the combination therapies of the present invention comprise a ULK inhibitor and an additional therapeutic agent. In some embodiments, the ULK inhibitor inhibits ULK1. In some embodiments, the ULK inhibitor is a ULK1 specific inhibitor. In some embodiments, the ULK inhibitor inhibits both ULK1 and ULK2. In some embodiments, there is an additional therapeutic benefit when compared to treatment with the additional therapeutic agent alone. In some instances, the combination of the ULK inhibitor and the additional therapeutic agent shut down pathways of autophagy. In some instances, this allows for enhanced cell death in diseased tissue, as the diseased cells will not be able to rely on autophagic processes for survival once the pathway is shut off with a ULK inhibitor. In some embodiments, the addition of a ULK inhibitor allows for successful treatment of a disease that is otherwise refractory to treatment of the additional therapeutic agent by itself. In some embodiments, the addition of the ULK inhibitor enhances the efficacy of the additional therapeutic agent. In some embodiments, the addition of the ULK inhibitor has a synergistic effect with the additional therapeutic agent. In some embodiments, the additional therapeutic agent is a standard of care therapy.

In one aspect, provided herein, is a method of treating a disease or disorder with a ULK inhibitor and an additional therapeutic agent. In some embodiments, the method comprises administering to a subject in need thereof a therapeutically effective amount of a ULK inhibitor. In some embodiments, the method comprises administering to a subject in need thereof a therapeutically effective amount of a ULK inhibitor and a therapeutically effective amount of an additional therapeutic agent. In some embodiments, the additional therapeutic agent is not an mTOR inhibitor. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, the disease or disorder is cancer. In some embodiments, the disease or disorder is refractory cancer. In some embodiments, the cancer comprises a tumor. In some embodiments, the cancer is refractory to treatment with carboplatin. In some embodiments, the cancer is refractory to trametinib. In some embodiments, the cancer is refractory to an MEK inhibitor. In some embodiments, the cancer is refractory to a standard of care therapy.

In some embodiments, cancer is pancreatic cancer. In some embodiments, the cancer is lung cancer. In some embodiments, the lung cancer is non-small cell lung cancer. In some embodiments, the cancer is breast cancer.

In some embodiments, the additional therapeutic agent is carboplatin. In some embodiments, the additional therapeutic agent is a carboplatin analog. In some embodiments, the carboplatin analog is cisplatin or dicycloplatin.

In some embodiments, the additional therapeutic agent is an MEK inhibitor. In some embodiments, the additional therapeutic agent is trametinib. In some embodiments, the MEK inhibitor is trametinib, cobimetinib, binimetinib, or selumetinib.

In some embodiments, the additional therapeutic agent is gemcitabine. In some embodiments, the additional therapeutic agent is a nucleoside analog.

In some embodiments, the additional therapeutic agent is a PARP inhibitor. In some embodiments, the PARP inhibitor is olaparib, rucaparib, niraparib, or talazoparib. In some embodiments, the additional therapeutic agent is olaparib, rucaparib, niraparib, or talazoparib.

In some embodiments, the additional therapeutic agent is erlotinib, gefitinib, osimertinib, or crizotinib. In some embodiments, the additional therapeutic agent is anastrozole, exemestane, letrozole, or tamoxifen. In some embodiments, the additional therapeutic agent is gemcitabine, everolimus, erlotinib, or sunitinib. In some embodiments, the additional therapeutic agent is erlotinib, gefitinib, osimertinib, crizotinib, pemetrexed, docetaxol, or pembroluzimab.

In some embodiments, the cancer is pancreatic cancer and the additional therapeutic agent is trametinb. In some embodiments, the cancer is pancreatic cancer and the additional therapeutic agent is an MEK inhibitor. In some embodiments, the MEK inhibitor is trametinib, cobimetinib, binimetinib, or selumetinib. In some embodiments, the cancer is pancreatic cancer and the additional therapeutic agent is gemcitabine. In some embodiments, the cancer is pancreatic cancer and the additional therapeutic agent is a nucleoside analog. In some embodiments, the cancer is pancreatic cancer and the additional therapeutic agent is gemcitabine, everolimus, erlotinib, or sunitinib. In some embodiments, the additional therapeutic agent is FOLFIRINOX, gemcitabine, or gemcitabine/abraxane. In some embodiments, the additional therapeutic agent is capeditabine, leucovorin, nab-paclitaxel, nanoliposomal irinotecan, gemcitabine/nab-paclitaxel, pembrolizumab, or cisplatin. In some embodiments, the additional therapeutic agent is capeditabine, leucovorin, nab-paclitaxel, nanoliposomal irinotecan, gemcitabine/nab-paclitaxel, pembrolizumab, or cisplatin. In some embodiments, the pancreatic cancer is PDAC. In some embodiments, the subject with pancreatic cancer comprises a mutation in at least one of SMAD4, p16/CDKM2A, or BRCA2.

In some embodiments, the cancer is breast cancer. In some embodiments, the cancer is breast cancer and the additional therapeutic agent is anastrozole, exemestane, letrozole, or tamoxifen. In some embodiments, the cancer is breast cancer and the additional therapeutic agent is a PARP inhibitor. In some embodiments, the PARP inhibitor is olaparib, rucaparib, niraparib, or talazoparib. In some embodiments, the breast cancer is TNBC.

In some embodiments, the cancer is lung cancer and the additional therapeutic agent is carboplatin. In some embodiments, the cancer is lung cancer and the additional therapeutic agent is a carboplatin analog. In some embodiments, the cancer is non-small cell lung cancer and the additional therapeutic agent is carboplatin. In some embodiments, the cancer is non-small cell lung cancer and the additional therapeutic agent is a carboplatin analog. In some embodiments, the carboplatin analog is cisplatin or dicycloplatin. In some embodiments, the cancer is lung cancer and the additional therapeutic agent is erlotinib, gefitinib, osimertinib, or crizotinib. In some embodiments, the cancer is non-small cell lung cancer and the additional therapeutic agent is erlotinib, gefitinib, osimertinib, or crizotinib. In some embodiments, the cancer is lung cancer and the additional therapeutic agent is pemetrexed, docetaxol, or pembroluzimab. In some embodiments, the cancer is non-small cell lung cancer and the additional therapeutic agent is pemetrexed, docetaxol, or pembroluzimab. In some embodiments, the cancer is lung cancer and the additional therapeutic agent is gemcitabine, bortexomib, trastuzumab, vinorelbine, doxorubicin, irinotecan, temsirolimus, sunitinib, nivolumab, or bevacizumab. In some embodiments, the cancer is lung cancer and the additional therapeutic agent is carboplatin/gemcitabine, carboplatin/paclitaxel/cetuximua, cisplatin/pemetrexed, cisplatin/docetaxel, cisplatin/docetaxel/bevacizumab, everolimus/nab-paclitaxel, or tremelimumab/durvalumab. In some embodiments, the cancer is non-small cell lung cancer and the additional therapeutic agent is gemcitabine, bortexomib, trastuzumab, vinorelbine, doxorubicin, irinotecan, temsirolimus, sunitinib, nivolumab, or bevacizumab. In some embodiments, the cancer is non-small cell lung cancer and the additional therapeutic agent is carboplatin/gemcitabine, carboplatin/paclitaxel/cetuximua, cisplatin/pemetrexed, cisplatin/docetaxel, cisplatin/docetaxel/bevacizumab, everolimus/nab-paclitaxel, or tremelimumab/durvalumab. In some embodiments, the subject with lung cancer comprises a mutation in KRAS, PTEN, TSC1, TSC2, PIk3CA, P53, STK11 (a.k.a. LKB1), KEAP1, NRF2, ALK4, GNAS or EGFR

In some embodiments, the additional therapeutic agent was previously administered to the subject without a ULK inhibitor. In some embodiments, the additional therapeutic agent induces a cytostatic response. In some embodiments, the additional therapeutic agent induces a cytostatic response when administered without a ULK inhibitor. In some embodiments, the additional therapeutic agent induces a cytostatic response in disease tissue. In some embodiments, the additional therapeutic agent induces a cytostatic response in the diseased tissue when the additional therapeutic agent was administered without a ULK inhibitor.

In some embodiments, the subject is treated with the additional therapeutic agent prior to treatment with the ULK inhibitor. In some embodiments, treatment with the additional therapeutic agent is ceased prior to administration of the ULK inhibitor. In some embodiments, treatment with the additional therapeutic agent produces a cytostatic response in diseased tissue.

In some embodiments, the ULK inhibitor and the additional therapeutic agent are administered concomitantly. In some embodiments, the ULK inhibitor and the additional therapeutic agent are administered together at the start of treatment.

In some embodiments, the disease or disorder is characterized by abnormal autophagy. In some embodiments, the abnormal autophagy is therapeutically induced. In some embodiments, the disease or disorder characterized by abnormal autophagy is refractory. In some embodiments, the disease or disorder characterized by abnormal autophagy is refractory to a standard of care therapy. In some embodiments, the disease or disorder characterized by abnormal autophagy is resistant to a standard of care therapy.

In some embodiments, administering a ULK inhibitor slows progression of the disease or disorder. In some embodiments, administering a ULK inhibitor slows progression of the disease or disorder when compared to administration of the additional therapeutic agent without the ULK inhibitor. In some embodiments, administering a ULK inhibitor slows progression of the disease or disorder by at least 10%, at least 20%, at least 30%, at least 40%, at least 50%, at least 60%, at least 70%, at least 80%, at least 90%, or at least 95%. In some embodiments, administering a ULK inhibitor slows the progression of the disease or disorder by at least 10%, at least 20%, at least 30%, at least 40%, at least 50%, at least 60%, at least 70%, at least 80%, at least 90%, or at least 95% when compared to administration of the additional therapeutic agent without the ULK inhibitor. In some embodiments, progression of the disease or disorder comprises growth of a tumor.

In some embodiments, administering a ULK inhibitor enhances the efficacy of the additional therapeutic agent by at least 10%, at least 20%, at least 30%, at least 40%, at least 50%, at least 60%, at least 70%, at least 80%, at least 90%, or at least 95%. In some embodiments, administering a ULK inhibitor enhances the efficacy of the additional therapeutic agent by at least 10%, at least 20%, at least 30%, at least 40%, at least 50%, at least 60%, at least 70%, at least 80%, at least 90%, or at least 95% when compared to administration of the additional therapeutic agent without the ULK inhibitor. In some embodiments, the efficacy is measured by a change in the rate of tumor growth. In some embodiments, efficacy is measured by reduction of tumor volume. In some embodiments, progression is measured by tumor growth. In some embodiments, administering a ULK inhibitor arrests cancer cell growth. In some embodiments, administering a ULK inhibitor reduces tumor volume.

In some embodiments, the method of treatment comprises decreasing phosphorylation of ATG13 in the subject. In some embodiments, the method comprises degrading ATG13 in diseased tissue of the subject. In some embodiments, administering a ULK inhibitor causes degradation of ATG13.

In some embodiments, the subject comprises a mutation in KRAS, PTEN, TSC1, TSC2, PIk3CA, P53, STK11 (a.k.a. LKB1), KEAP1, NRF2, or EGFR. In some embodiments, the subject comprises a mutation in at least one of SMAD4, p16/CDKM2A, or BRCA2. In some embodiments, the subject comprises a mutation in at least one of ALK4 or GNAS. In some embodiments, the subject comprises a mutation in at least one of KRAS, PTEN, TSC1, TSC2, PIk3CA, P53, STK11 (a.k.a. LKB1), KEAP1, NRF2, EGFR, SMAD4, p16/CDKM2A, BRCA2, ALK4, or GNAS.

### Compounds

In one aspect, disclosed herein are compounds that function as ULK inhibitors.

In certain embodiments, the ULK inhibitor is at least one selected from the group consisting of a 2-(substituted)amino-4-(substituted)amino-5-halo-pyrimidine; 2-(substituted)amino-4-(substituted) amino-5-(halo)alkyl-pyrimidine; 2-(substituted)amino-4-(substituted)oxo-5-halo-pyrimidine; 2-(substituted)amino-4-(substituted)oxo-5-(halo)alkyl-pyrimidine; 2-(substituted)amino-4-(substituted)thio-5-halo-pyrimidine; and 2-(substituted)amino-4-(substituted)thio-5-(halo)alkyl-pyrimidine; or a pharmaceutically acceptable salt thereof.

Also disclosed herein are ULK inhibitors, or pharmaceutically acceptable salts thereof, having a structure of:

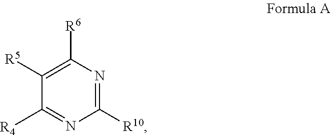

wherein in Formula A:
R¹⁰ is selected from the group consisting of: halogen; —OR¹¹ wherein R¹¹ is H, optionally substituted aryl, or optionally substituted heteroaryl; —NR¹R² wherein R¹ and R² are each individually selected from the group consisting of H, —C(═O)R¹², where R¹² is C₁-C₆ alkyl or C₁-C₆ cycloalkyl, optionally substituted aryl, optionally substituted heteroaryl, optionally substituted cycloalkyl, and optionally substituted alkyl, or NR¹R² together form a heterocycle; or R⁴ and R¹⁰ together form a cyclic structure;
R⁴ is selected from the group consisting of optionally substituted amino, optionally substituted aryloxy, optionally substituted heteroaryloxy, optionally substituted alkoxy, N-heterocyclic, optionally substituted thiol, optionally substituted alkyl, hydroxyl and halogen;
R⁵ is selected from the group consisting of H, hydroxyl, optionally substituted alkyl (e.g., fluoroalkyl), halogen, optionally substituted alkoxy, or optionally substituted aryl, optionally substituted carboxyl, cyano, and nitro, or R⁵ and R⁶ together form a cyclic structure; and
R⁶ is H, halogen, or haloalkyl.

In some embodiments, R¹⁰ is —OR¹¹. In some embodiments, R¹¹ is optionally substituted aryl or optionally substituted heteroaryl. In some embodiments, R¹¹ is an optionally substituted phenyl ring fused with a 5- or 6-membered cycloalkyl, heterocycloalkyl, aryl, or heteroaryl ring, wherein the 5- or 6-membered ring is independently optionally substituted. In some embodiments, R¹¹ is optionally substituted napthyl, optionally substituted tetrahydronapthyl, optionally substituted quinolyl, optionally substituted indolyl, or optionally substituted tetrahydroquinolyl. In some embodiments, R¹¹ is optionally substituted napthyl, optionally substituted tetrahydronapthyl, optionally substituted quinolyl, optionally substituted indolyl, or optionally substituted tetrahydroquinolyl, wherein the napthyl, tetrahydronapthyl, quinolyl, indolyl, or tetrahydroquinolyl is optionally substituted with —OH, —NH₂, alkyl, halogen, or alkoxy. In some embodiments, R¹¹ is napthyl optionally substituted with —OH, —NH₂, alkyl, halogen, or alkoxy. R¹¹ is unsubstituted napthyl, unsubstituted tetrahydronapthyl, unsubstituted quinolyl, unsubstituted indolyl, or unsubstituted tetrahydroquinolyl. In some embodiments, R¹¹ is optionally substituted phenyl. In some embodiments, R¹¹ is phenyl optionally substituted with —OH, —NH₂, alkyl, halogen, or alkoxy.

In some embodiments, R¹⁰ is —NR¹R². In some embodiments, R¹ and R² together form a heterocycle. R¹ and R² together form an unsubstituted 4-8 membered heterocycle.

In some embodiments, R¹ is H or —C₁-C₆ alkyl. In some embodiments, R¹ is H or —CH₃. In some embodiments, R¹ is H.

In some embodiments, R² is optionally substituted alkyl or optionally substituted cycloalkyl. In some embodiments, R² is optionally substituted alkyl. In some embodiments, R² is optionally substituted cycloalkyl. In some embodiments, R² is unsubstituted cycloalkyl. In some embodiments, R² is cyclopropyl, cyclobutyl, or cyclopentyl. In some embodiments, R² is unsubstituted cyclopropyl, unsubstituted cyclobutyl, or unsubstituted cyclopentyl.

In some embodiments, R² is optionally substituted aryl or heteroaryl. R² is optionally substituted phenyl. In some embodiments, R² is phenyl optionally substituted with one or more substituents selected from alkyl, alkoxy, haloalkoxy, halogen, —S-alkyl, phenoxy, hydroxy, morpholinyl. R² is alkoxy substituted phenyl. R² is optionally substituted heteroaryl. In some embodiments, R² is optionally substituted pyridyl, optionally substituted pyrazinyl, optionally substituted pyrimidinyl, optionally substituted pyridazinyl, optionally substituted indolyl, optionally substituted benzimdazolyl, optionally substituted benzotriazolyl, or optionally substituted 7-azaindolyl. In some embodiments, R² is optionally substituted pyridyl, optionally substituted pyrazinyl, optionally substituted pyrimidinyl, optionally substituted pyridazinyl, optionally substituted indolyl, optionally substituted benzimdazolyl, optionally substituted benzotriazolyl, or optionally substituted 7-azaindolyl, wherein the pyridyl, pyrazinyl, pyrmidinyl, pyridazinyl, indolyl, benzimidazolyl, benzotriazolyl, or 7-azaindolyl is optionally substituted with one more substituent selected from —OH, —NH₂, alkyl, halogen, or alkoxy. In some embodiments, R² is optionally substituted 5- or 6-membered heteroaryl. In some embodiments, R² is an optionally substituted fused heteroaryl. In some embodiments, R² is an optionally substituted bicyclic fused ring system that contains at least one nitrogen atom. In some embodiments, R² is selected from the group consisting of

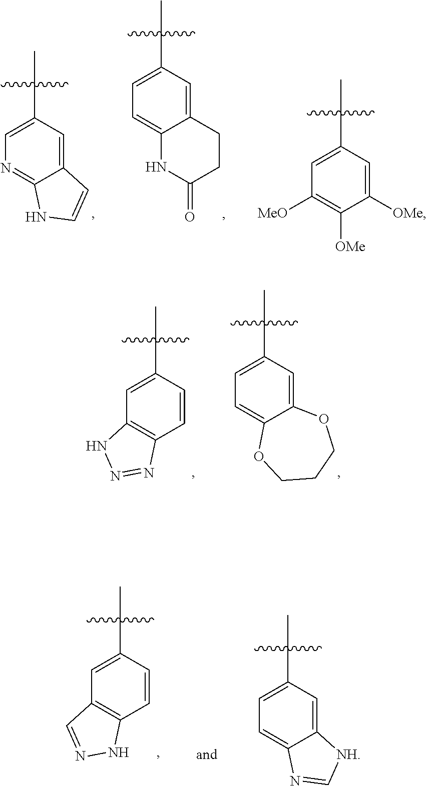

In some embodiments, R⁴ is selected from the group consisting of optionally substituted amino, optionally substituted aryloxy, optionally substituted heteroaryloxy, and optionally substituted alkoxy.

In some embodiments, R⁴ is optionally substituted aryloxy or optionally substituted heteroaryloxy. In some embodiments, R⁴ is aryloxy or heteroarylxy, wherein the aryloxy or heteroaryloxy is optionally substituted with one or more substituents selected from —C(═O)NH(C₁-C₆ alkyl), alkoxy, halogen, —NH₂, NH(C₁-C₆ alkyl), —NH—[(C═O)C₁-C₆ alkyl], nitrile, —S—C₁-C₆ alykl, morpholino, C₁-C₆ alkyl, —SO₂—(C₁-C₆ alkyl), or haloalkyl. In some embodiments, R⁴ is selected from the group consisting of phenoxy, (C₁-C₆)alkoxy, and —O—(N-alkylbenzamide), particularly —O—(N—(C₁-C₆)alkylbenzamide). In some embodiments, R⁴ is:

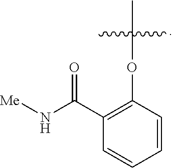 [SMILES: *Oc1ccccc1C(=O)NC]

In some embodiments, R⁴ is —S(C₁-C₆)alkyl, —O(C₁-C₆ alkyl), or —O(C₃-C₈ cycloalkyl). In some embodiments, R⁴ is —S(C₁-C₆)alkyl. In some embodiments, R⁴ is —O(C₁-C₆ alkyl). In some embodiments, R⁴ is —O(C₁-C₆ alkyl).

In some embodiments, R⁴ is —NR⁷R⁸, wherein R⁷ and R⁸ are each individually selected from the group consisting of H, optionally substituted aryl, optionally substituted heteroaryl, cycloalkyl, and optionally substituted alkyl, or NR⁷R⁸ together form a heterocycle. In some embodiments, R⁷ and R⁸ together form an unsubstituted 4-8 membered heterocycle. In some embodiments, R⁷ and R⁸ together form a heterocycle.

In some embodiments, R⁷ and R⁸ are each independently selected from H and C₁-C₆ alkyl with one or two substituents selected from —OH, —OMe, —C(═O)OMe, —C(═O)OH, —NH₂, —NHMe, —N(Me)₂, —NHCH₂CH₂OH, and cyclopropyl.

In some embodiments, R⁷ is H or —CH₃. In some embodiments, R⁷ is H.

In some embodiments, R⁸ is optionally substituted aryl or optionally substituted heteroaryl. In some embodiments, R⁸ is optionally substituted phenyl or optionally substituted pyridyl. In some embodiments, R⁸ is optionally substituted phenyl or optionally substituted pyridyl, wherein the phenyl or pyridyl is optionally substituted with —C(═O)NH(C₁-C₆ alkyl), alkoxy, halogen, —NH₂, NH(C₁-C₆ alkyl), —NH—[(C═O)C₁-C₆ alkyl], nitrile, —S—C₁-C₆ alykl, morpholino, C₁-C₆ alkyl, —SO₂—(C₁-C₆ alkyl), or haloalkyl.

In some embodiments, R⁸ is phenyl optionally substituted with —C(═O)NH(C₁-C₆ alkyl), alkoxy, halogen, —NH₂, NH(C₁-C₆ alkyl), —NH(C═O)C₁-C₆ alkyl, nitrile, —S—C₁-C₆ alkyl, morpholinyl, C₁-C₆ alkyl, —SO₂—(C₁-C₆ alkyl), or haloalkyl. In some embodiments, R⁸ is phenyl optionally substituted with —C(═O)NH(C₁-C₆ alkyl), alkoxy, or halogen. In some embodiments, R⁸ is phenyl optionally substituted with —C(═O)NHMe or —OMe.

In some embodiments, R⁸ is pyridyl is optionally substituted with —C(═O)NH(C₁-C₆ alkyl), alkoxy, halogen, —NH₂, NH(C₁-C₆ alkyl), —NH(C═O)C₁-C₆ alkyl, nitrile, —S—C₁-C₆ alkyl, morpholinyl, C₁-C₆ alkyl, —SO₂—(C₁-C₆ alkyl), or haloalkyl. In some embodiments, R⁸ is pyridyl optionally substituted with —C(═O)NH(C₁-C₆ alkyl), alkoxy, or halogen. In some embodiments, R⁸ is pyridyl optionally substituted with —C(═O)NHMe or —OMe.

In some embodiments, R⁸ is cycloalkyl. In some embodiments, R⁸ is optionally substituted C₃-C₈ cycloalkyl. In some embodiments, R⁸ is unsubstituted C₃-C₈ cycloalkyl. In some embodiments, R⁸ is unsubstituted C₃-C₆ cycloalkyl. In some embodiments, R⁸ is cyclopropyl or cyclobutyl.

In some embodiments, R⁵ is H, halogen, C₁-C₃ fluoroalkyl, or cyano. In some embodiments, R⁵ is Br, Cl, or —CF₃. In some embodiments, R⁵ is Cl. In some embodiments, R⁵ is Br. In some embodiments, R⁵ is —CF₃.

In some embodiments, R⁶ is H, —CF₃, or F. In some embodiments, R⁶ is H or F. In some embodiments, R⁶ is H. In some embodiments, R⁶ is F.

Also disclosed herein are ULK inhibitors or pharmaceutically acceptable salts thereof, having a structure of:

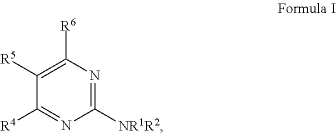

wherein in Formula I;
R¹ and R² are each individually selected from the group consisting of H, optionally substituted aryl, optionally substituted heteroaryl, optionally substituted cycloalkyl, and optionally substituted alkyl, or NR¹R² together form a heterocycle;
R⁴ is selected from the group consisting of optionally substituted amino, optionally substituted aryloxy, optionally substituted heteroaryloxy, optionally substituted alkoxy, N-heterocyclic, optionally substituted thiol, and optionally substituted alkyl;
R⁵ is selected from the group consisting of H, hydroxyl, optionally substituted alkyl (e.g., fluoroalkyl such as trifluoromethyl), halogen, optionally substituted alkoxy, and optionally substituted aryl; and
R⁶ is H or fluorine; or a pharmaceutically acceptable salt thereof.

In some embodiments, R¹ and R² together form a heterocycle. R¹ and R² together form an unsubstituted 4-8 membered heterocycle.

In some embodiments, R¹ is H or —C₁-C₆ alkyl. In some embodiments, R¹ is H or —CH₃. In some embodiments, R¹ is H.

In some embodiments, R² is optionally substituted alkyl or optionally substituted cycloalkyl. In some embodiments, R² is optionally substituted alkyl. In some embodiments, R² is optionally substituted cycloalkyl. In some embodiments, R² is unsubstituted cycloalkyl. In some embodiments, R² is cyclopropyl, cyclobutyl, or cyclopentyl. In some embodiments, R² is unsubstituted cyclopropyl, unsubstituted cyclobutyl, or unsubstituted cyclopentyl.

In some embodiments, R² is optionally substituted aryl or heteroaryl. R² is optionally substituted phenyl. In some embodiments, R² is phenyl optionally substituted with one or more substituents selected from alkyl, alkoxy, haloalkoxy, halogen, —S-alkyl, phenoxy, hydroxy, morpholinyl. R² is alkoxy substituted phenyl. R² is optionally substituted heteroaryl. In some embodiments, R² is optionally substituted pyridyl, optionally substituted pyrazinyl, optionally substituted pyrimidinyl, optionally substituted pyridazinyl, optionally substituted indolyl, optionally substituted benzimidazolyl, optionally substituted benzotriazolyl, or optionally substituted 7-azaindolyl. In some embodiments, R² is optionally substituted pyridyl, optionally substituted pyrazinyl, optionally substituted pyrimidinyl, optionally substituted pyridazinyl, optionally substituted indolyl, optionally substituted benzimidazolyl, optionally substituted benzotriazolyl, or optionally substituted 7-azaindolyl, wherein the pyridyl, pyrazinyl, pyrmidinyl, pyridazinyl, indolyl, benzimidazolyl, benzotriazolyl, or 7-azaindolyl is optionally substituted with one more substituent selected from —OH, —NH₂, alkyl, halogen, or alkoxy. In some embodiments, R² is optionally substituted 5- or 6-membered heteroaryl. In some embodiments, R² is an optionally substituted fused heteroaryl. In some embodiments, R² is an optionally substituted bicyclic fused ring system that contains at least one nitrogen atom. In some embodiments, R² is selected from the group consisting of

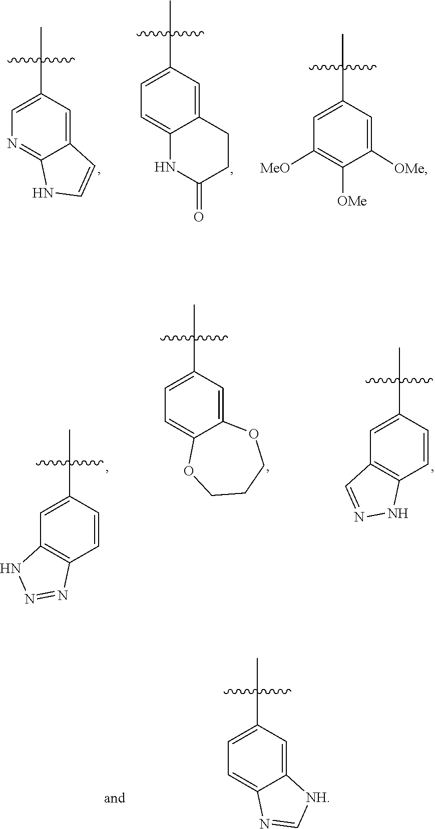

In some embodiments, R⁴ is selected from the group consisting of optionally substituted amino, optionally substituted aryloxy, optionally substituted heteroaryloxy, and optionally substituted alkoxy.

In some embodiments, R⁴ is optionally substituted aryloxy or optionally substituted heteroaryloxy. In some embodiments, R⁴ is aryloxy or heteroaryloxy, wherein the aryloxy or heteroaryloxy is optionally substituted with one or more substituents selected from —C(═O)NH(C₁-C₆ alkyl), alkoxy, halogen, —NH₂, NH(C₁-C₆ alkyl), —NH—[(C═O)C₁-C₆ alkyl], nitrile, —S—C₁-C₆ alykl, morpholino, C₁-C₆ alkyl, —SO₂—(C₁-C₆ alkyl), or haloalkyl. In some embodiments, R⁴ is selected from the group consisting of phenoxy, (C₁-C₆)alkoxy, and —O—(N-alkylbenzamide), particularly —O—(N—(C₁-C₆)alkylbenzamide). In some embodiments, R⁴ is:

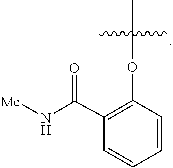

In some embodiments, R⁴ is —S(C₁-C₆)alkyl, —O(C₁-C₆ alkyl), or —O(C₃-C₈ cycloalkyl). In some embodiments, R⁴ is —S(C₁-C₆)alkyl. In some embodiments, R⁴ is —O(C₁-C₆ alkyl). In some embodiments, R⁴ is —O(C₁-C₆ alkyl).

In some embodiments, R⁴ is —NR⁷R⁸, wherein R⁷ and R⁸ are each individually selected from the group consisting of H, optionally substituted aryl, optionally substituted heteroaryl, cycloalkyl, and optionally substituted alkyl, or NR⁷R⁸ together form a heterocycle. In some embodiments, R⁷ and R⁸ together form an unsubstituted 4-8 membered heterocycle. In some embodiments, R⁷ and R⁸ together form a heterocycle.

In some embodiments, R⁷ and R⁸ are each independently selected from H and C₁-C₆ alkyl with one or two substituents selected from —OH, —OMe, —C(═O)OMe, —C(═O)OH, —NH₂, —NHMe, —N(Me)₂, —NHCH₂CH₂OH, and cyclopropyl.

In some embodiments, R⁷ is H or —CH₃. In some embodiments, R⁷ is H.

In some embodiments, R⁸ is optionally substituted aryl or optionally substituted heteroaryl. In some embodiments, R⁸ is optionally substituted phenyl or optionally substituted pyridyl. In some embodiments, R⁸ is optionally substituted phenyl or optionally substituted pyridyl, wherein the phenyl or pyridyl is optionally substituted with —C(═O)NH(C₁-C₆ alkyl), alkoxy, halogen, —NH₂, NH(C₁-C₆ alkyl), —NH—[(C═O)C₁-C₆ alkyl], nitrile, —S—C₁-C₆ alykl, morpholino, C₁-C₆ alkyl, —SO₂—(C₁-C₆ alkyl), or haloalkyl.

In some embodiments, R⁸ is phenyl optionally substituted with —C(═O)NH(C₁-C₆ alkyl), alkoxy, halogen, —NH₂, NH(C₁-C₆ alkyl), —NH(C═O)C₁-C₆ alkyl, nitrile, —S—C₁-C₆ alkyl, morpholinyl, C₁-C₆ alkyl, —SO₂—(C₁-C₆ alkyl), or haloalkyl. In some embodiments, R⁸ is phenyl optionally substituted with —C(═O)NH(C₁-C₆ alkyl), alkoxy, or halogen. In some embodiments, R⁸ is phenyl optionally substituted with —C(═O)NHMe or —OMe.

In some embodiments, R⁸ is pyridyl is optionally substituted with —C(═O)NH(C₁-C₆alkyl), alkoxy, halogen, —NH₂, NH(C₁-C₆ alkyl), —NH(C═O)C₁-C₆ alkyl, nitrile, —S—C₁-C₆ alkyl, morpholinyl, C₁-C₆ alkyl, —SO₂—(C₁-C₆ alkyl), or haloalkyl. In some embodiments, R⁸ is pyridyl optionally substituted with —C(═O)NH(C₁-C₆ alkyl), alkoxy, or halogen. In some embodiments, R⁸ is pyridyl optionally substituted with —C(═O)NHMe or —OMe.

In some embodiments, R⁸ is cycloalkyl. In some embodiments, R⁸ is optionally substituted C₃-C₈ cycloalkyl. In some embodiments, R⁸ is unsubstituted C₃-C₈ cycloalkyl. In some embodiments, R⁸ is unsubstituted C₃-C₆ cycloalkyl. In some embodiments, R⁸ is cyclopropyl or cyclobutyl.

In some embodiments, R⁵ is H, halogen, C₁-C₃ fluoroalkyl, or cyano. In some embodiments, R⁵ is Br, Cl, or —CF₃. In some embodiments, R⁵ is Cl. In some embodiments, R⁵ is Br. In some embodiments, R⁵ is —CF₃.

In some embodiments, R⁶ is H, —CF₃, or F. In some embodiments, R⁶ is H or F. In some embodiments, R⁶ is H. In some embodiments, R⁶ is F.

In some embodiments, R¹ is H and R² is not H. In other embodiments, R¹ is H and R² is an optionally substituted fused heteroaryl or an optionally substituted aryl. The optionally substituted fused heteroaryl, for example, may be a bicyclic fused ring system that include at least one nitrogen heteroatom. In some embodiments, R¹ is H and R² is an optionally substituted bicyclic fused ring system that includes at least one heteroatom. In some embodiments, R¹ is H and R² is an optionally substituted bicyclic fused ring system that includes at least one nitrogen heteroatoms. In some embodiments, R¹ is H and R² is an optionally substituted bicyclic fused ring system that includes at least two nitrogen heteroatoms. In some embodiments, R¹ is H and R² is an optionally substituted bicyclic fused ring system that includes at least two oxygen heteroatoms. The optionally substituted aryl, for example, may be a substituted or unsubstituted phenyl. The phenyl, for example, may be substituted with at least one alkoxy, preferably (C₁-C₆)alkoxy.

In some embodiments, R¹ is H and R² is selected from the group consisting of:

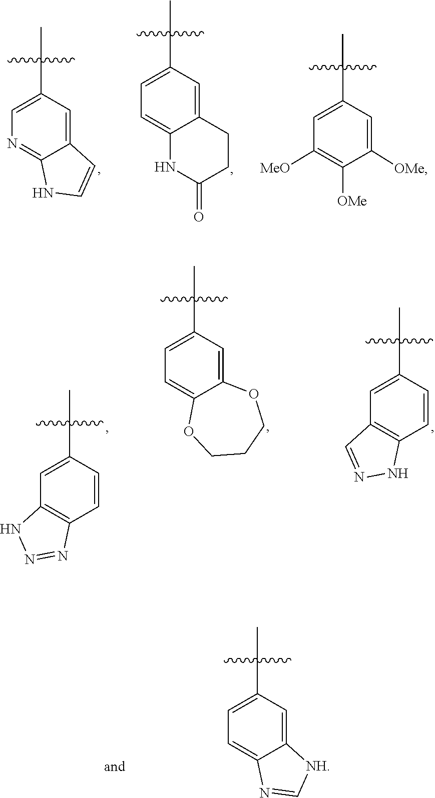

In some embodiments, R⁴ is selected from the group consisting of optionally substituted amino, optionally substituted aryloxy, optionally substituted heteroaryloxy, and optionally substituted alkoxy.

In some embodiments, R⁴ is selected from the group consisting of optionally substituted aryloxy, optionally substituted heteroaryloxy, and optionally substituted alkoxy. In particular embodiments, R⁴ is selected from the group consisting of optionally substituted phenoxy and optionally substituted alkoxy. In particular embodiments, R⁴ is selected from the group consisting of phenoxy, (C₁-C₆)alkoxy, and —O—(N-alkylbenzamide), particularly —O—(N—(C₁-C₆)alkylbenzamide). In particular embodiments, R⁴ is

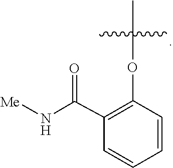

In some embodiments, R⁴ is —NR⁷R⁸, wherein R⁷ and R⁸ are each individually selected from the group consisting of H, optionally substituted aryl, optionally substituted heteroaryl, cycloalkyl, and optionally substituted alkyl, or NR⁷R⁸ together form a heterocycle. In some embodiments, R⁷ is H and R⁸ is N-alkylbenzamide, particularly N—(C₁-C₆)alkylbenzamide. In some embodiments, R⁷ is H and R⁸ is phenyl. In some embodiments, R⁷ is H and R⁸ is alkoxy-substituted phenyl, particularly (C₁-C₆)alkoxy. In some embodiments, R⁷ is H and R⁸ is cyclopropyl. In some embodiments, R⁷ is H and R⁸ is cyclobutyl. In some embodiments, R⁷ is H and R⁸ is alkoxyalkyl, particularly (C₁-C₆)alkoxy(C₁-C₆)alkyl. In some embodiments, R⁷ is H and R⁸ is haloalkyl. In some embodiments, R⁷ is H and R⁸ is optionally substituted acyl. In some embodiments, R⁴ is —NH₂. In some embodiments, R⁴ —OH.

In some embodiments, R⁵ is haloalkyl, particularly —CF₃. In some embodiments, R⁵ is Br. In some embodiments, R⁵ is Cl.

In some embodiments, R² is a fused heteroaryl ring and R⁴ is —NR⁷R⁸, wherein R⁷ is H and R⁸ is a fused heteroaryl ring. In particular embodiments, R² is selected from the group consisting of:

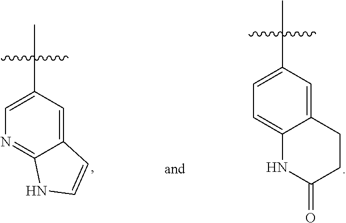 [SMILES: *c1cnc2[nH]ccc2c1 ; *c1ccc2c(c1)CCC(=O)N2]

In particular embodiments, R⁸ is:

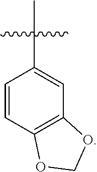 [SMILES: *c1ccc2c(c1)OCO2]

In some embodiments, R¹ is H or —CH₃; R² is alkoxy substituted phenyl; R⁴ is —NR⁷R⁸, wherein, R⁷ is H or —CH₃ and R⁸ is R⁸ is phenyl optionally substituted with —C(═O)NHMe or —OMe; R⁵ is Br, Cl, or —CF₃, and R⁶ is H or F.

In some embodiments, R¹ is H or —CH₃; R² is selected from the group consisting of

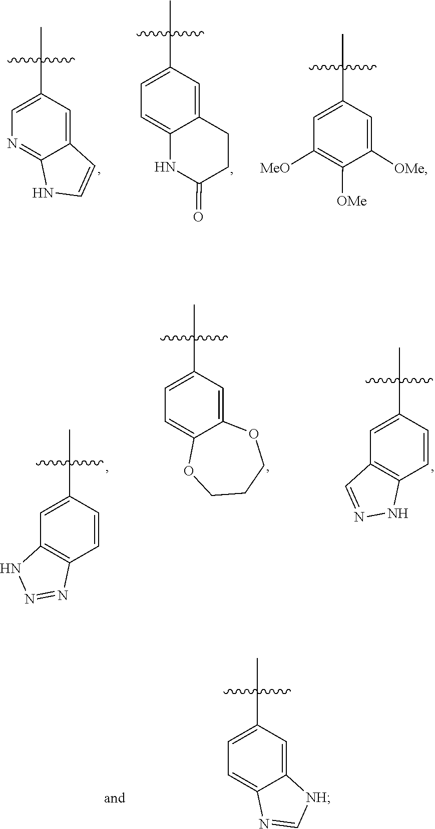

R⁴ is —NR⁷R⁸ wherein R⁷ is H or —CH₃ and R⁸ is phenyl optionally substituted with —C(═O)NHMe or —OMe; R⁵ is Br, Cl, or —CF₃, and R⁶ is H or F.

Any combination of the groups described above for the various variables is contemplated herein. Throughout the specification, groups and substituents thereof are chosen by one skilled in the field to provide stable moieties and compounds.

Illustrative compounds are shown in Table 1 (along with their respective IC₅₀ values for ULK1 inhibition assay). IC₅₀s are presented in μM, with A representing IC₅₀<0.2 μM, B representing 0.2 μM<IC₅₀<2 μM, and C representing IC₅₀>2 μM. An * notes tested as a mixture of regioisomers.

TABLE 1

| Example | Structure | IC₅₀ |
|---|---|---|
| 12 | 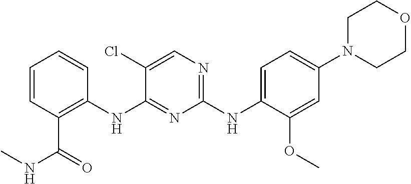 [SMILES: CNC(=O)c1ccccc1Nc1nc(Nc2ccc(N3CCOCC3)cc2OC)ncc1Cl] | A |
| 13 | 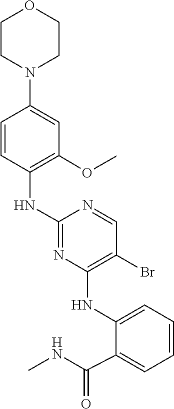 [SMILES: CNC(=O)c1ccccc1Nc1nc(Nc2ccc(N3CCOCC3)cc2OC)ncc1Br] | B |
| 14 | 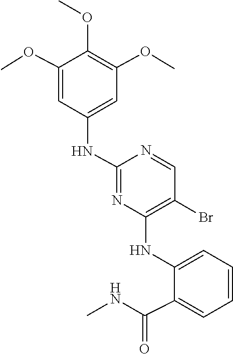 [SMILES: CNC(=O)c1ccccc1Nc1nc(Nc2cc(OC)c(OC)c(OC)c2)ncc1Br] | A |
| 15 | 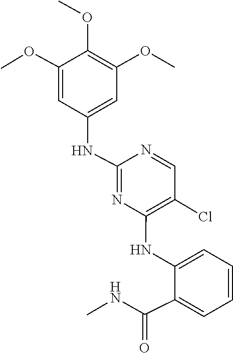 [SMILES: CNC(=O)c1ccccc1Nc1nc(Nc2cc(OC)c(OC)c(OC)c2)ncc1Cl] | A |
| 16 | 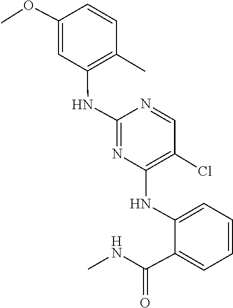 [SMILES: CNC(=O)c1ccccc1Nc1nc(Nc2cc(OC)ccc2C)ncc1Cl] | A |
| 17 | 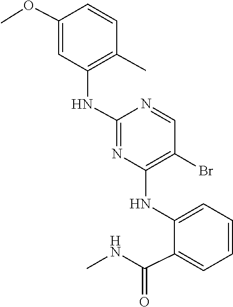 [SMILES: CNC(=O)c1ccccc1Nc1nc(Nc2cc(OC)ccc2C)ncc1Br] | A |
| 18 | 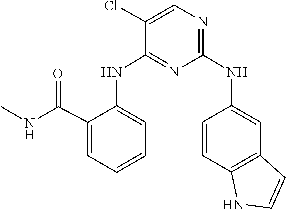 [SMILES: CNC(=O)c1ccccc1Nc1nc(Nc2ccc3[nH]ccc3c2)ncc1Cl] | B |
| 19 | 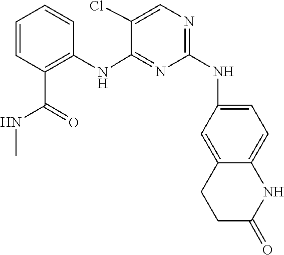 [SMILES: CNC(=O)c1ccccc1Nc1nc(Nc2ccc3c(c2)CCC(=O)N3)ncc1Cl] | A |
| 20 | 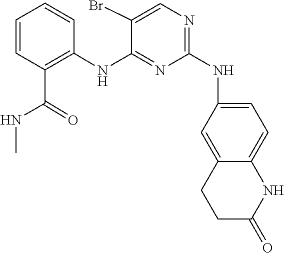 [SMILES: CNC(=O)c1ccccc1Nc1nc(Nc2ccc3c(c2)CCC(=O)N3)ncc1Br] | A |
| 21 | 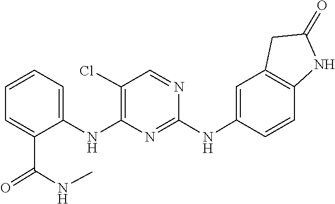 [SMILES: CNC(=O)c1ccccc1Nc1nc(Nc2ccc3c(c2)CC(=O)N3)ncc1Cl] | A |
| 22 | 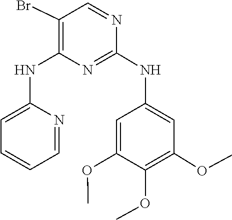 [SMILES: COc1cc(Nc2ncc(Br)c(Nc3ccccn3)n2)cc(OC)c1OC] | A |
| 23 | 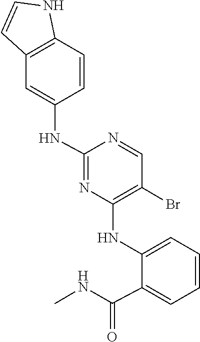 [SMILES: CNC(=O)c1ccccc1Nc1nc(Nc2ccc3[nH]ccc3c2)ncc1Br] | A |
| 24 | 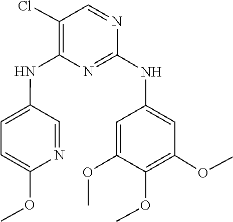 | A |
| 25 | 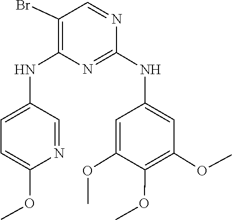 | A |
| 26 | 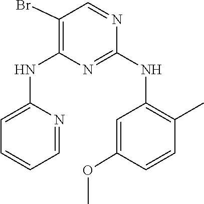 [SMILES: COc1ccc(C)c(Nc2ncc(Br)c(Nc3ccccn3)n2)c1] | B |
| 27 | 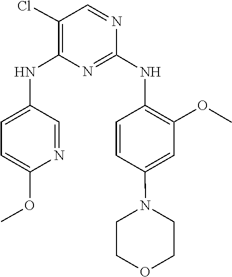 | C |
| 28 | 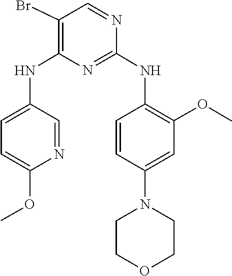 | B |
| 29 | 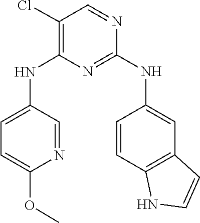 [SMILES: COc1ccc(Nc2nc(Nc3ccc4[nH]ccc4c3)ncc2Cl)cn1] | B |
| 30 | 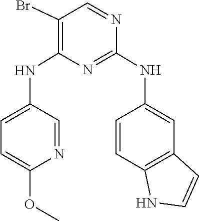 [SMILES: COc1ccc(Nc2nc(Nc3ccc4[nH]ccc4c3)ncc2Br)cn1] | A |
| 31 | 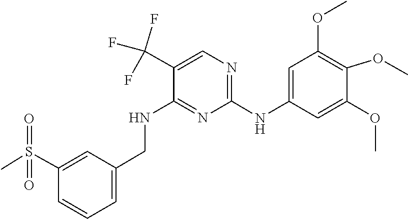 | A |
| 32 | 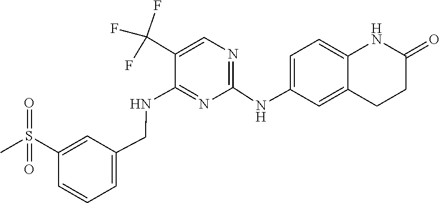 [SMILES: CS(=O)(=O)c1cccc(CNc2nc(Nc3ccc4c(c3)CCC(=O)N4)ncc2C(F)(F)F)c1] | B |
| 33 | 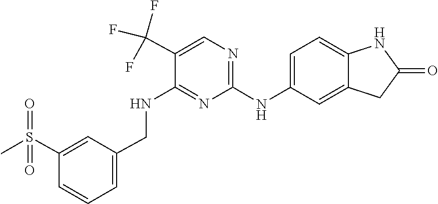 [SMILES: CS(=O)(=O)c1cccc(CNc2nc(Nc3ccc4c(c3)CC(=O)N4)ncc2C(F)(F)F)c1] | B |
| 34 | 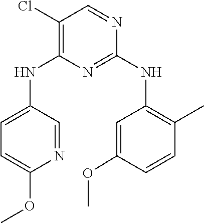 | C |
| 35 | 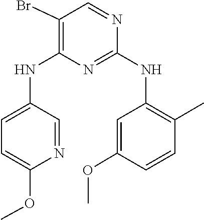 | C |
| 36 | 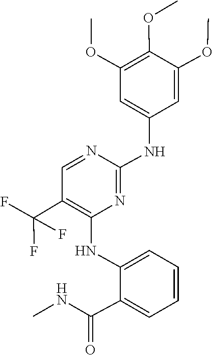 [SMILES: CNC(=O)c1ccccc1Nc1nc(Nc2cc(OC)c(OC)c(OC)c2)ncc1C(F)(F)F] | C |
| 37 | 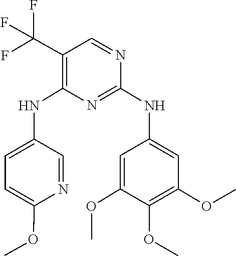 | A |
| 38 | 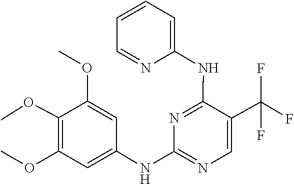 [SMILES: COc1cc(Nc2ncc(C(F)(F)F)c(Nc3ccccn3)n2)cc(OC)c1OC] | B |
| 39 | 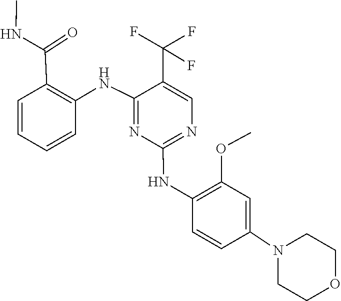 [SMILES: CNC(=O)c1ccccc1Nc1nc(Nc2ccc(N3CCOCC3)cc2OC)ncc1C(F)(F)F] | A |
| 40 | 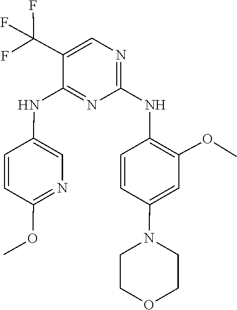 | C |
| 41 | 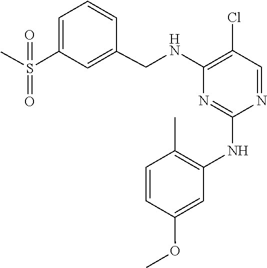 [SMILES: COc1ccc(C)c(Nc2ncc(Cl)c(NCc3cccc(S(C)(=O)=O)c3)n2)c1] | C |
| 42 | 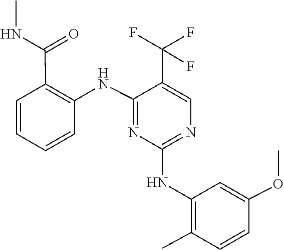 | A |
| 43 | 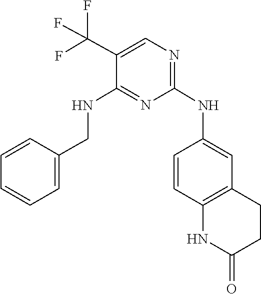 [SMILES: O=C1CCc2cc(Nc3ncc(C(F)(F)F)c(NCc4ccccc4)n3)ccc2N1] | A |
| 44 | 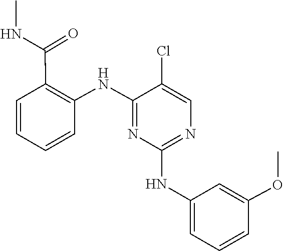 [SMILES: CNC(=O)c1ccccc1Nc1nc(Nc2cccc(OC)c2)ncc1Cl] | A |
| 45 | 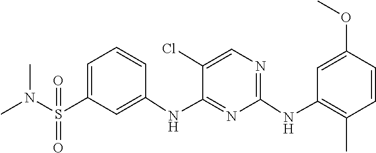 [SMILES: COc1ccc(C)c(Nc2ncc(Cl)c(Nc3cccc(S(=O)(=O)N(C)C)c3)n2)c1] | C |
| 46 | 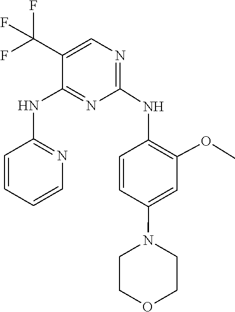 [SMILES: COc1cc(N2CCOCC2)ccc1Nc1ncc(C(F)(F)F)c(Nc2ccccn2)n1] | B |
| 48 | 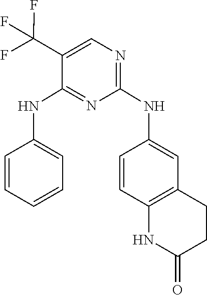 [SMILES: O=C1CCc2cc(Nc3ncc(C(F)(F)F)c(Nc4ccccc4)n3)ccc2N1] | A |
| 49 | 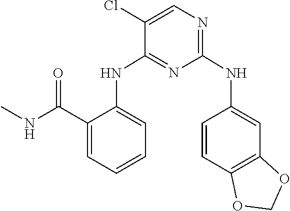 [SMILES: CNC(=O)c1ccccc1Nc1nc(Nc2ccc3c(c2)OCO3)ncc1Cl] | A |
| 50 | 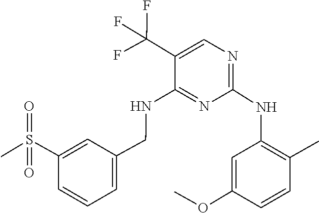 | C |
| 51 | 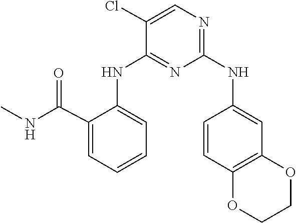 [SMILES: CNC(=O)c1ccccc1Nc1nc(Nc2ccc3c(c2)OCCO3)ncc1Cl] | A |
| 52 | 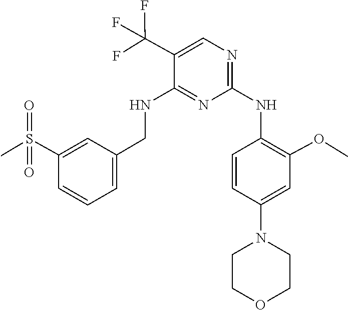 | C |
| 53 | 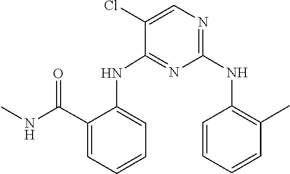 | C |
| 54 | 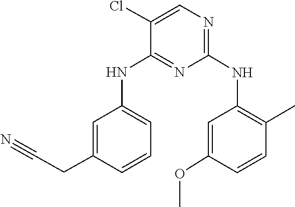 [SMILES: COc1ccc(C)c(Nc2ncc(Cl)c(Nc3cccc(CC#N)c3)n2)c1] | C |
| 55 | 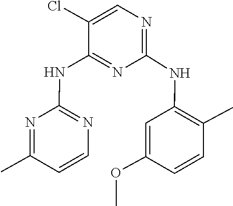 | C |
| 56 | 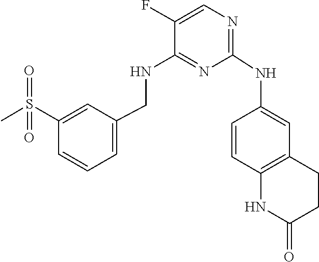 [SMILES: CS(=O)(=O)c1cccc(CNc2nc(Nc3ccc4c(c3)CCC(=O)N4)ncc2F)c1] | C |
| 57 | 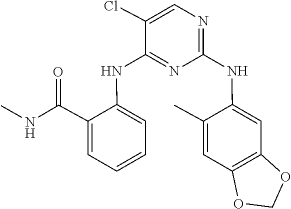 [SMILES: CNC(=O)c1ccccc1Nc1nc(Nc2cc3c(cc2C)OCO3)ncc1Cl] | A |
| 58 | 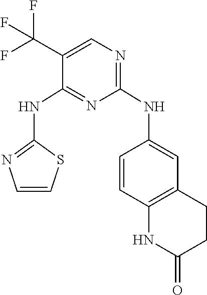 [SMILES: O=C1CCc2cc(Nc3ncc(C(F)(F)F)c(Nc4nccs4)n3)ccc2N1] | C |
| 60 | 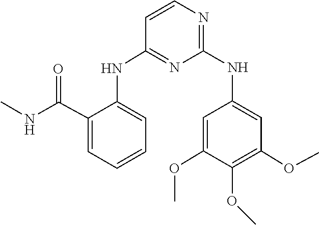 | A |
| 61 | 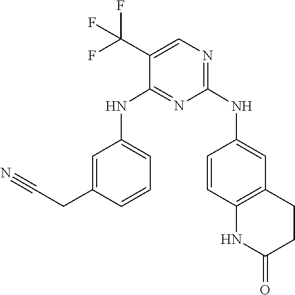 [SMILES: N#CCc1cccc(Nc2nc(Nc3ccc4c(c3)CCC(=O)N4)ncc2C(F)(F)F)c1] | A |
| 62 | 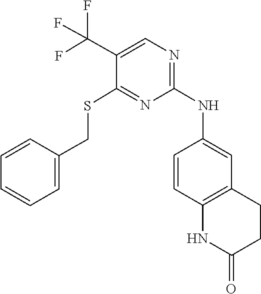 [SMILES: O=C1CCc2cc(Nc3ncc(C(F)(F)F)c(SCc4ccccc4)n3)ccc2N1] | A |
| 64 | 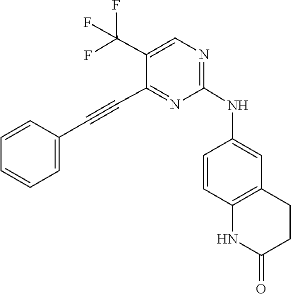 [SMILES: O=C1CCc2cc(Nc3ncc(C(F)(F)F)c(C#Cc4ccccc4)n3)ccc2N1] | C |
| 66 | 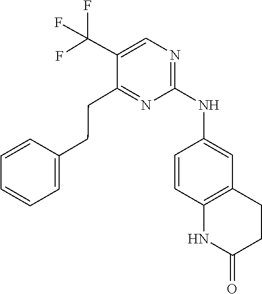 [SMILES: O=C1CCc2cc(Nc3ncc(C(F)(F)F)c(CCc4ccccc4)n3)ccc2N1] | B |
| 67 | 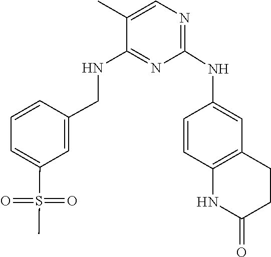 [SMILES: Cc1cnc(Nc2ccc3c(c2)CCC(=O)N3)nc1NCc1cccc(S(C)(=O)=O)c1] | C |
| 69 | 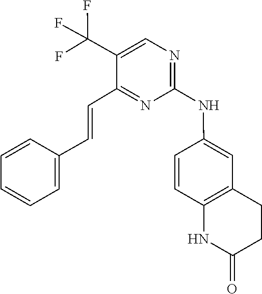 [SMILES: O=C1CCc2cc(Nc3ncc(C(F)(F)F)c(/C=C/c4ccccc4)n3)ccc2N1] | A |
| 71 | 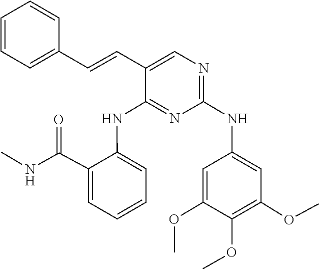 [SMILES: CNC(=O)c1ccccc1Nc1nc(Nc2cc(OC)c(OC)c(OC)c2)ncc1/C=C/c1ccccc1] | A |
| 72 | 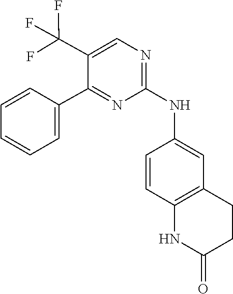 [SMILES: O=C1CCc2cc(Nc3ncc(C(F)(F)F)c(-c4ccccc4)n3)ccc2N1] | C |
| 73 | 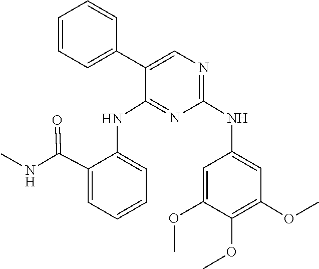 | A |
| 74 | 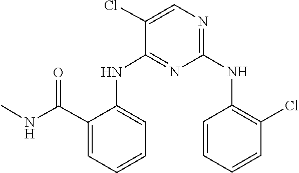 [SMILES: CNC(=O)c1ccccc1Nc1nc(Nc2ccccc2Cl)ncc1Cl] | B |
| 75 | 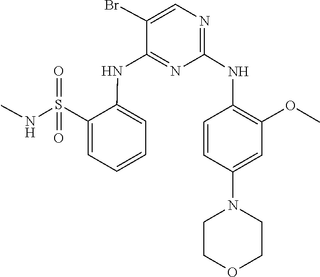 | B |
| 76 | 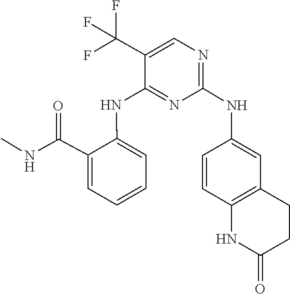 [SMILES: CNC(=O)c1ccccc1Nc1nc(Nc2ccc3c(c2)CCC(=O)N3)ncc1C(F)(F)F] | A |
| 78 | 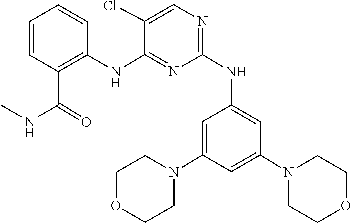 [SMILES: CNC(=O)c1ccccc1Nc1nc(Nc2cc(N3CCOCC3)cc(N3CCOCC3)c2)ncc1Cl] | B |
| 80 | 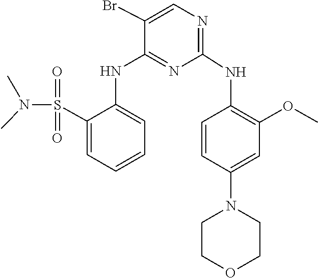 | A |
| 81 | 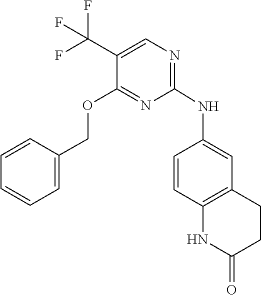 [SMILES: O=C1CCc2cc(Nc3ncc(C(F)(F)F)c(OCc4ccccc4)n3)ccc2N1] | A |
| 82 | 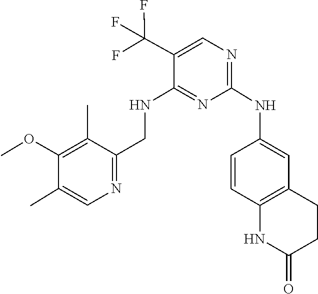 [SMILES: COc1c(C)cnc(CNc2nc(Nc3ccc4c(c3)CCC(=O)N4)ncc2C(F)(F)F)c1C] | A |
| 83 | 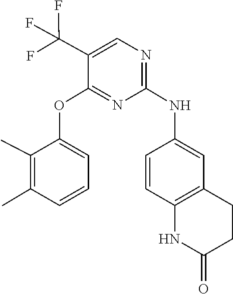 [SMILES: Cc1cccc(Oc2nc(Nc3ccc4c(c3)CCC(=O)N4)ncc2C(F)(F)F)c1C] | A |
| 87 | 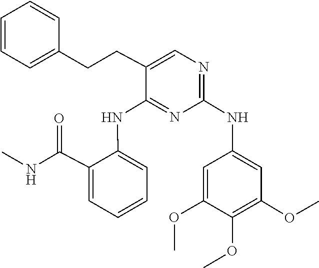 [SMILES: CNC(=O)c1ccccc1Nc1nc(Nc2cc(OC)c(OC)c(OC)c2)ncc1CCc1ccccc1] | B |
| 88 | 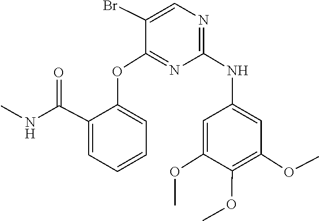 | A |
| 89 | 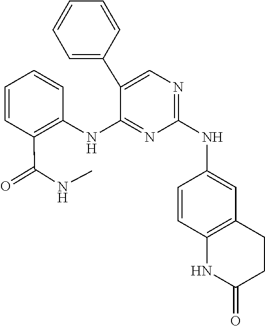 [SMILES: CNC(=O)c1ccccc1Nc1nc(Nc2ccc3c(c2)CCC(=O)N3)ncc1-c1ccccc1] | A |
| 90 | 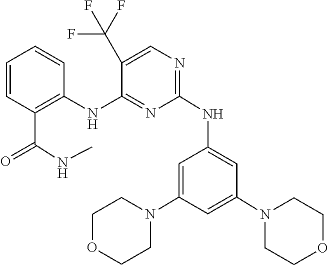 [SMILES: CNC(=O)c1ccccc1Nc1nc(Nc2cc(N3CCOCC3)cc(N3CCOCC3)c2)ncc1C(F)(F)F] | A |
| 92 | 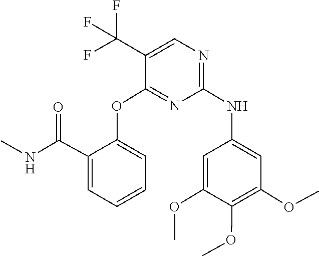 | A |
| 99 | 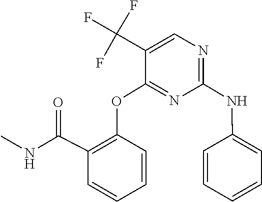 [SMILES: CNC(=O)c1ccccc1Oc1nc(Nc2ccccc2)ncc1C(F)(F)F] | C |
| 100 | 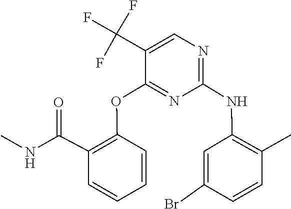 | C |
| 101 | 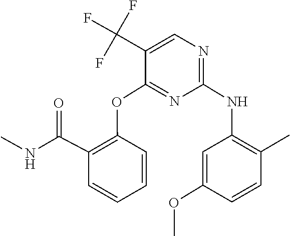 | C |
| 102 | 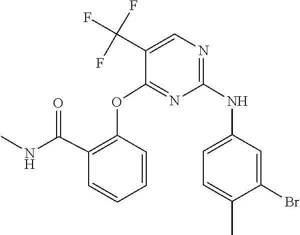 [SMILES: CNC(=O)c1ccccc1Oc1nc(Nc2ccc(C)c(Br)c2)ncc1C(F)(F)F] | C |
| 103 | 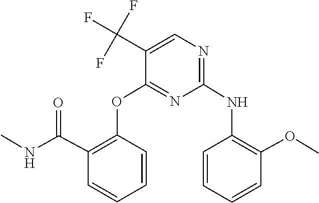 | C |
| 104 | 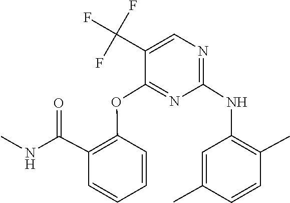 | C |
| 105a | 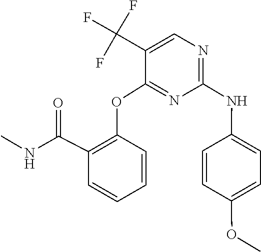 | C |
| 105b | 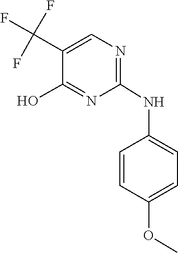 [SMILES: COc1ccc(Nc2ncc(C(F)(F)F)c(O)n2)cc1] | C |
| 106 | 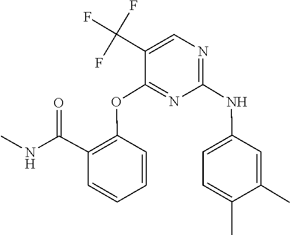 | B |
| 107 | 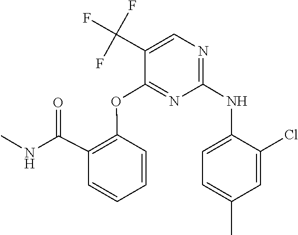 [SMILES: CNC(=O)c1ccccc1Oc1nc(Nc2ccc(C)cc2Cl)ncc1C(F)(F)F] | C |
| 108a | 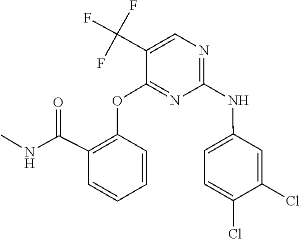 [SMILES: CNC(=O)c1ccccc1Oc1nc(Nc2ccc(Cl)c(Cl)c2)ncc1C(F)(F)F] | C |
| 108b | 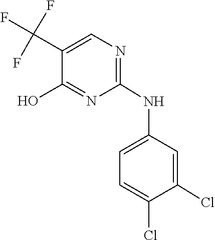 [SMILES: Oc1nc(Nc2ccc(Cl)c(Cl)c2)ncc1C(F)(F)F] | C |
| 109a | 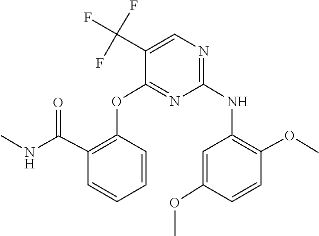 | C |
| 109b | 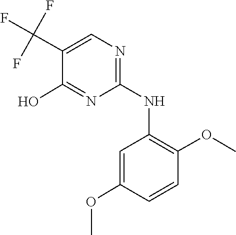 [SMILES: COc1ccc(OC)c(Nc2ncc(C(F)(F)F)c(O)n2)c1] | C |
| 110 | 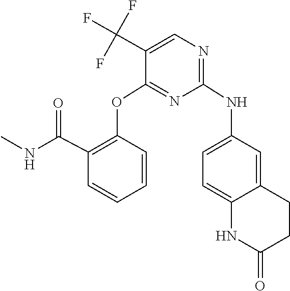 [SMILES: CNC(=O)c1ccccc1Oc1nc(Nc2ccc3c(c2)CCC(=O)N3)ncc1C(F)(F)F] | B |
| 111 | 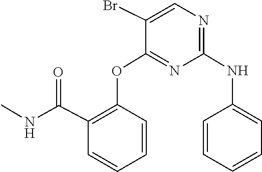 [SMILES: CNC(=O)c1ccccc1Oc1nc(Nc2ccccc2)ncc1Br] | C |
| 112 | 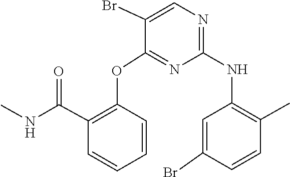 | C |
| 113 | 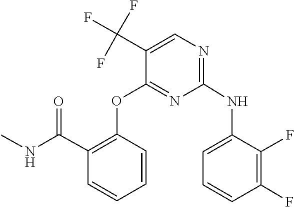 [SMILES: CNC(=O)c1ccccc1Oc1nc(Nc2cccc(F)c2F)ncc1C(F)(F)F] | C |
| 114 | 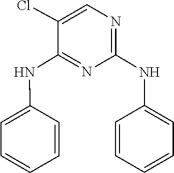 [SMILES: Clc1cnc(Nc2ccccc2)nc1Nc1ccccc1] | C |
| 115a | 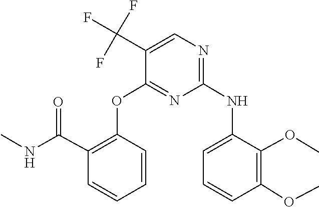 | C |
| 115b | 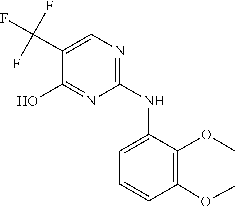 [SMILES: COc1cccc(Nc2ncc(C(F)(F)F)c(O)n2)c1OC] | C |
| 116a | 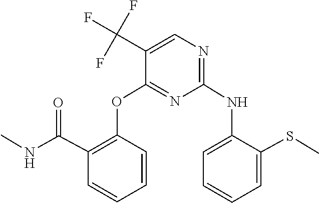 | C |
| 116b | 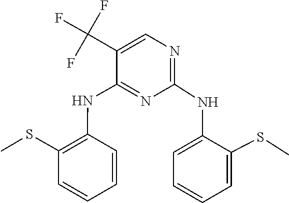 | C |
| 117 | 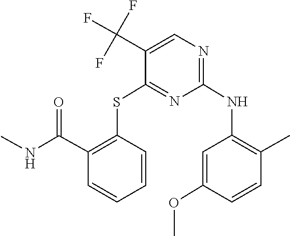 | C |
| 118a | 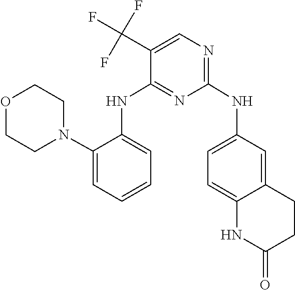 [SMILES: O=C1CCc2cc(Nc3ncc(C(F)(F)F)c(Nc4ccccc4N4CCOCC4)n3)ccc2N1] | A |
| 118b | 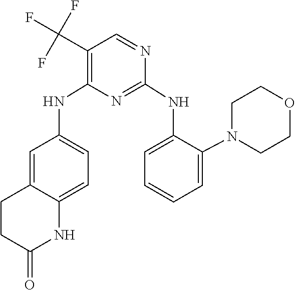 [SMILES: O=C1CCc2cc(Nc3nc(Nc4ccccc4N4CCOCC4)ncc3C(F)(F)F)ccc2N1] | C |
| 118c | 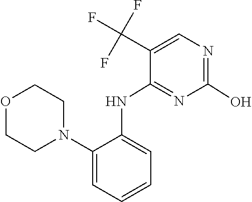 [SMILES: Oc1ncc(C(F)(F)F)c(Nc2ccccc2N2CCOCC2)n1] | C |
| 119 | 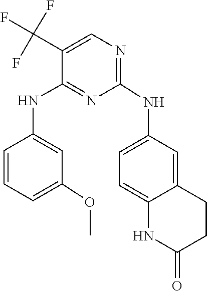 [SMILES: COc1cccc(Nc2nc(Nc3ccc4c(c3)CCC(=O)N4)ncc2C(F)(F)F)c1] | A |
| 120a | 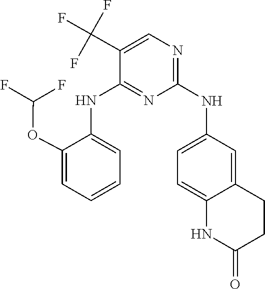 [SMILES: O=C1CCc2cc(Nc3ncc(C(F)(F)F)c(Nc4ccccc4OC(F)F)n3)ccc2N1] | A |
| 120b | 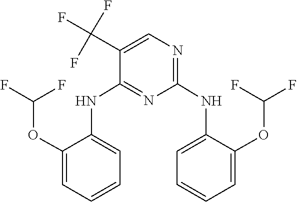 [SMILES: FC(F)Oc1ccccc1Nc1ncc(C(F)(F)F)c(Nc2ccccc2OC(F)F)n1] | B |
| 120c | 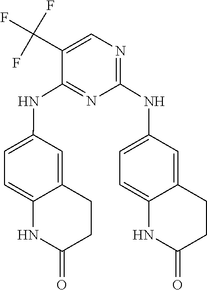 [SMILES: O=C1CCc2cc(Nc3ncc(C(F)(F)F)c(Nc4ccc5c(c4)CCC(=O)N5)n3)ccc2N1] | A |
| 121 | 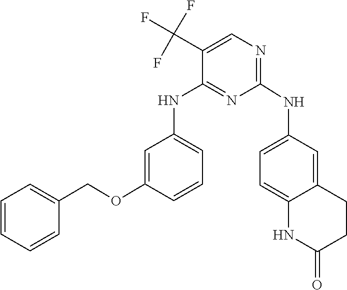 [SMILES: O=C1CCc2cc(Nc3ncc(C(F)(F)F)c(Nc4cccc(OCc5ccccc5)c4)n3)ccc2N1] | B |
| 122 | 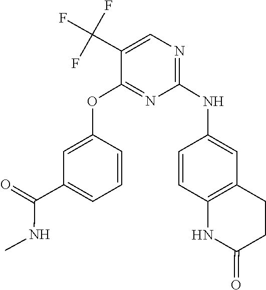 [SMILES: CNC(=O)c1cccc(Oc2nc(Nc3ccc4c(c3)CCC(=O)N4)ncc2C(F)(F)F)c1] | B |
| 123a | 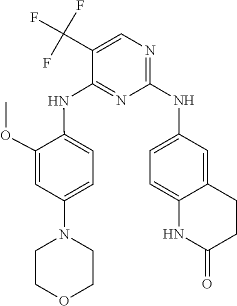 [SMILES: COc1cc(N2CCOCC2)ccc1Nc1nc(Nc2ccc3c(c2)CCC(=O)N3)ncc1C(F)(F)F] | A |
| 123b | 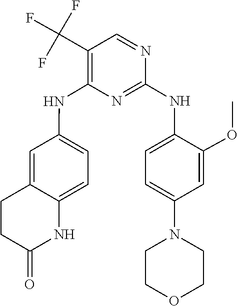 [SMILES: COc1cc(N2CCOCC2)ccc1Nc1ncc(C(F)(F)F)c(Nc2ccc3c(c2)CCC(=O)N3)n1] | A |
| 124* | 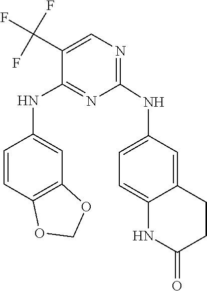 [SMILES: O=C1CCc2cc(Nc3ncc(C(F)(F)F)c(Nc4ccc5c(c4)OCO5)n3)ccc2N1] | A |
| 124c | 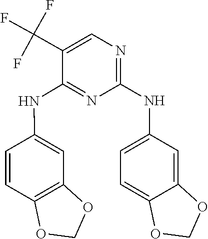 [SMILES: FC(F)(F)c1cnc(Nc2ccc3c(c2)OCO3)nc1Nc1ccc2c(c1)OCO2] | A |
| 125 | 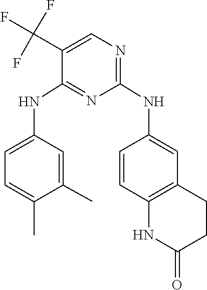 [SMILES: Cc1ccc(Nc2nc(Nc3ccc4c(c3)CCC(=O)N4)ncc2C(F)(F)F)cc1C] | A |
| 126a | 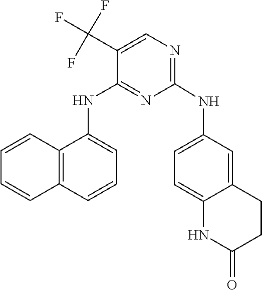 [SMILES: O=C1CCc2cc(Nc3ncc(C(F)(F)F)c(Nc4cccc5ccccc45)n3)ccc2N1] | B |
| 126b | 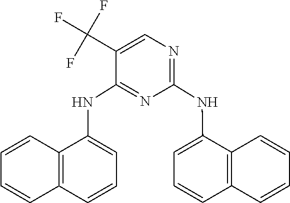 [SMILES: FC(F)(F)c1cnc(Nc2cccc3ccccc23)nc1Nc1cccc2ccccc12] | B |
| 127 | 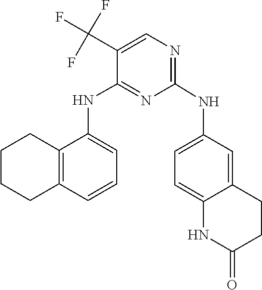 [SMILES: O=C1CCc2cc(Nc3ncc(C(F)(F)F)c(Nc4cccc5c4CCCC5)n3)ccc2N1] | B |
| 128 | 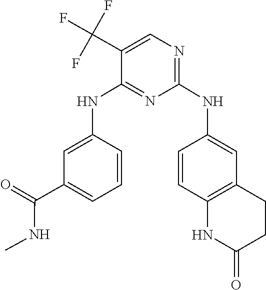 [SMILES: CNC(=O)c1cccc(Nc2nc(Nc3ccc4c(c3)CCC(=O)N4)ncc2C(F)(F)F)c1] | A |
| 129 | 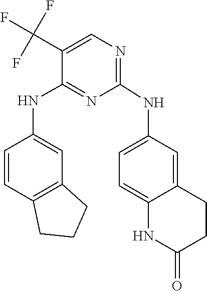 [SMILES: O=C1CCc2cc(Nc3ncc(C(F)(F)F)c(Nc4ccc5c(c4)CCC5)n3)ccc2N1] | A |
| 130a | 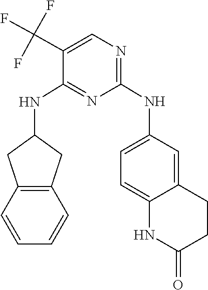 [SMILES: O=C1CCc2cc(Nc3ncc(C(F)(F)F)c(NC4Cc5ccccc5C4)n3)ccc2N1] | A |
| 130b | 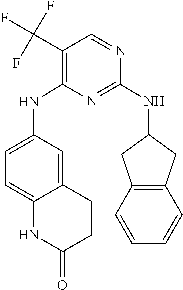 [SMILES: O=C1CCc2cc(Nc3nc(NC4Cc5ccccc5C4)ncc3C(F)(F)F)ccc2N1] | C |
| 131a | 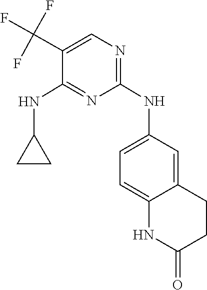 [SMILES: O=C1CCc2cc(Nc3ncc(C(F)(F)F)c(NC4CC4)n3)ccc2N1] | A |
| 131b | 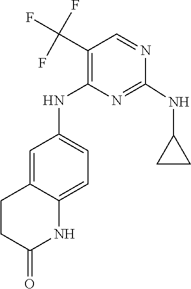 [SMILES: O=C1CCc2cc(Nc3nc(NC4CC4)ncc3C(F)(F)F)ccc2N1] | A |
| 132 | 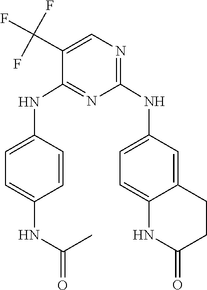 [SMILES: CC(=O)Nc1ccc(Nc2nc(Nc3ccc4c(c3)CCC(=O)N4)ncc2C(F)(F)F)cc1] | A |
| 133 | 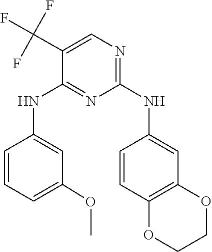 [SMILES: COc1cccc(Nc2nc(Nc3ccc4c(c3)OCCO4)ncc2C(F)(F)F)c1] | A |
| 134 | 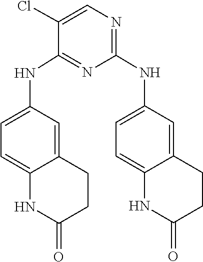 [SMILES: O=C1CCc2cc(Nc3ncc(Cl)c(Nc4ccc5c(c4)CCC(=O)N5)n3)ccc2N1] | A |
| 135 | 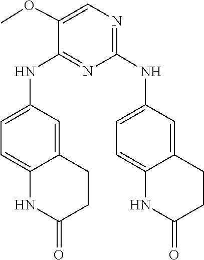 [SMILES: COc1cnc(Nc2ccc3c(c2)CCC(=O)N3)nc1Nc1ccc2c(c1)CCC(=O)N2] | C |
| 136* | 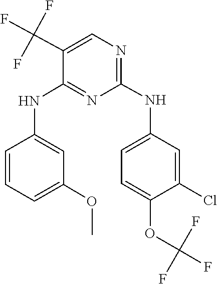 [SMILES: COc1cccc(Nc2nc(Nc3ccc(OC(F)(F)F)c(Cl)c3)ncc2C(F)(F)F)c1] | C |
| 137* | 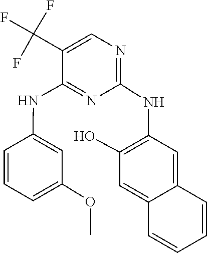 [SMILES: COc1cccc(Nc2nc(Nc3cc4ccccc4cc3O)ncc2C(F)(F)F)c1] | B |
| 138* | 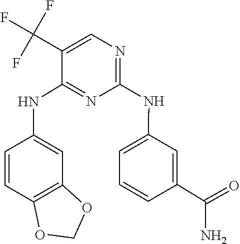 [SMILES: NC(=O)c1cccc(Nc2ncc(C(F)(F)F)c(Nc3ccc4c(c3)OCO4)n2)c1] | A |
| 139* | 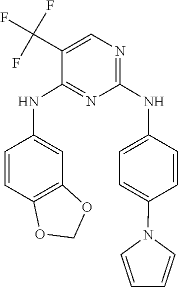 [SMILES: FC(F)(F)c1cnc(Nc2ccc(-n3cccc3)cc2)nc1Nc1ccc2c(c1)OCO2] | A |
| 140* | 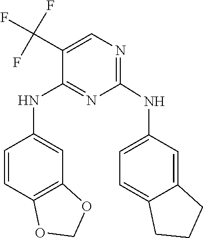 [SMILES: FC(F)(F)c1cnc(Nc2ccc3c(c2)CCC3)nc1Nc1ccc2c(c1)OCO2] | A |
| 141a | 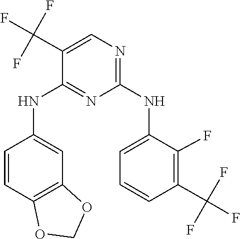 [SMILES: Fc1c(Nc2ncc(C(F)(F)F)c(Nc3ccc4c(c3)OCO4)n2)cccc1C(F)(F)F] | C |
| 141b | 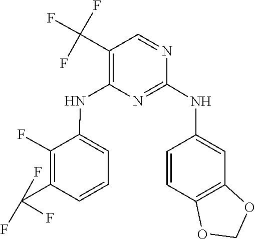 [SMILES: Fc1c(Nc2nc(Nc3ccc4c(c3)OCO4)ncc2C(F)(F)F)cccc1C(F)(F)F] | A |
| 142 | 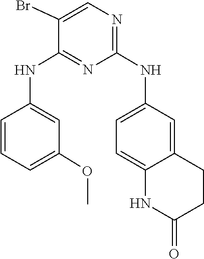 [SMILES: COc1cccc(Nc2nc(Nc3ccc4c(c3)CCC(=O)N4)ncc2Br)c1] | A |
| 143a | 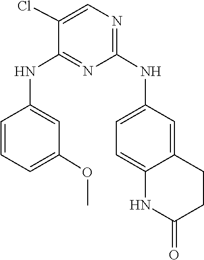 [SMILES: COc1cccc(Nc2nc(Nc3ccc4c(c3)CCC(=O)N4)ncc2Cl)c1] | A |
| 143b | 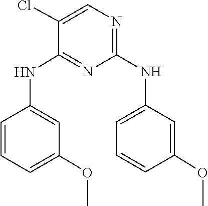 [SMILES: COc1cccc(Nc2ncc(Cl)c(Nc3cccc(OC)c3)n2)c1] | A |
| 144* | 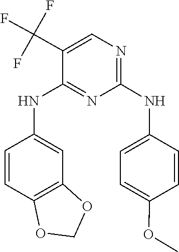 [SMILES: COc1ccc(Nc2ncc(C(F)(F)F)c(Nc3ccc4c(c3)OCO4)n2)cc1] | A |
| 145* | 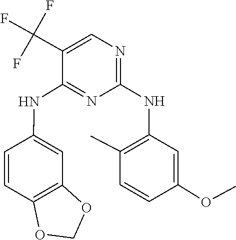 [SMILES: COc1ccc(C)c(Nc2ncc(C(F)(F)F)c(Nc3ccc4c(c3)OCO4)n2)c1] | B |
| 146* | 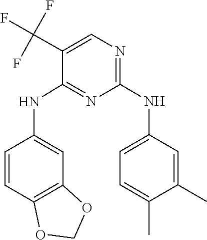 [SMILES: Cc1ccc(Nc2ncc(C(F)(F)F)c(Nc3ccc4c(c3)OCO4)n2)cc1C] | B |
| 147* | 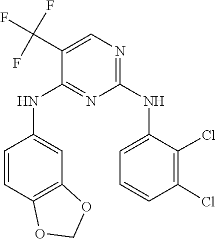 [SMILES: FC(F)(F)c1cnc(Nc2cccc(Cl)c2Cl)nc1Nc1ccc2c(c1)OCO2] | C |
| 148* | 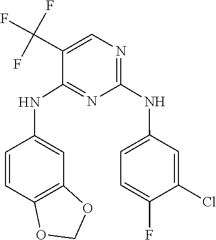 [SMILES: Fc1ccc(Nc2ncc(C(F)(F)F)c(Nc3ccc4c(c3)OCO4)n2)cc1Cl] | B |
| 149* | 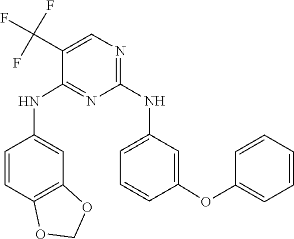 [SMILES: FC(F)(F)c1cnc(Nc2cccc(Oc3ccccc3)c2)nc1Nc1ccc2c(c1)OCO2] | C |
| 150a | 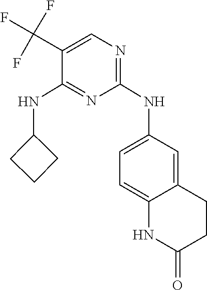 [SMILES: O=C1CCc2cc(Nc3ncc(C(F)(F)F)c(NC4CCC4)n3)ccc2N1] | A |
| 150b | 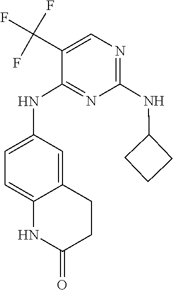 [SMILES: O=C1CCc2cc(Nc3nc(NC4CCC4)ncc3C(F)(F)F)ccc2N1] | A |
| 151a | 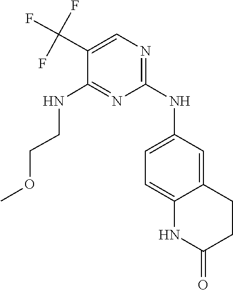 [SMILES: COCCNc1nc(Nc2ccc3c(c2)CCC(=O)N3)ncc1C(F)(F)F] | A |
| 151b | 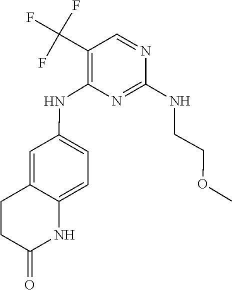 [SMILES: COCCNc1ncc(C(F)(F)F)c(Nc2ccc3c(c2)CCC(=O)N3)n1] | A |
| 152a | 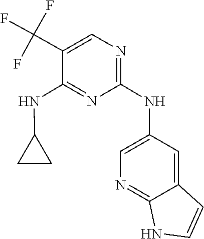 [SMILES: FC(F)(F)c1cnc(Nc2cnc3[nH]ccc3c2)nc1NC1CC1] | A |
| 152b | 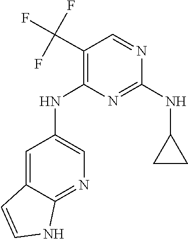 [SMILES: FC(F)(F)c1cnc(NC2CC2)nc1Nc1cnc2[nH]ccc2c1] | B |
| 153 | 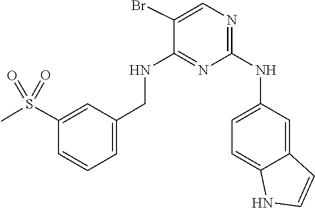 [SMILES: CS(=O)(=O)c1cccc(CNc2nc(Nc3ccc4[nH]ccc4c3)ncc2Br)c1] | C |
| 154 | 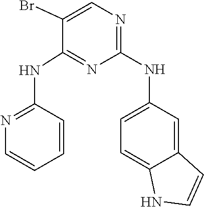 [SMILES: Brc1cnc(Nc2ccc3[nH]ccc3c2)nc1Nc1ccccn1] | A |
| 155 | 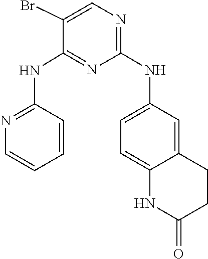 [SMILES: O=C1CCc2cc(Nc3ncc(Br)c(Nc4ccccn4)n3)ccc2N1] | A |
| 156 | 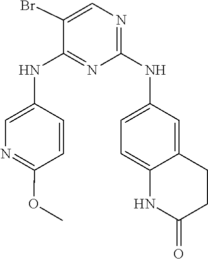 [SMILES: COc1ccc(Nc2nc(Nc3ccc4c(c3)CCC(=O)N4)ncc2Br)cn1] | A |
| 157* | 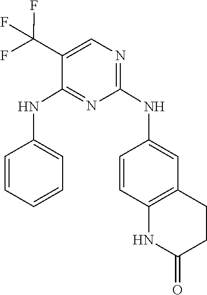 [SMILES: O=C1CCc2cc(Nc3ncc(C(F)(F)F)c(Nc4ccccc4)n3)ccc2N1] | A |
| 158a | 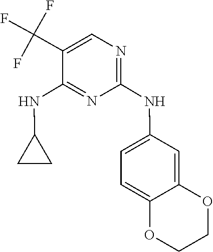 [SMILES: FC(F)(F)c1cnc(Nc2ccc3c(c2)OCCO3)nc1NC1CC1] | A |
| 158b | 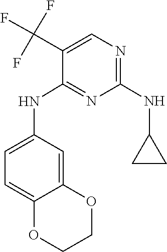 [SMILES: FC(F)(F)c1cnc(NC2CC2)nc1Nc1ccc2c(c1)OCCO2] | A |
| 159 | 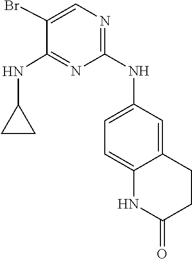 [SMILES: O=C1CCc2cc(Nc3ncc(Br)c(NC4CC4)n3)ccc2N1] | A |
| 160a | 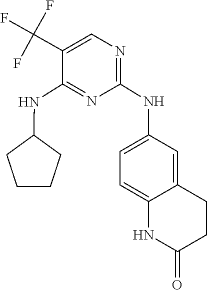 [SMILES: O=C1CCc2cc(Nc3ncc(C(F)(F)F)c(NC4CCCC4)n3)ccc2N1] | A |
| 160b | 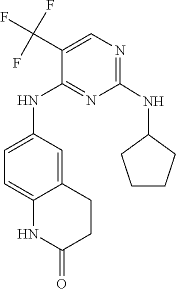 [SMILES: O=C1CCc2cc(Nc3nc(NC4CCCC4)ncc3C(F)(F)F)ccc2N1] | B |
| 161a | 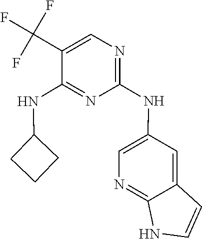 [SMILES: FC(F)(F)c1cnc(Nc2cnc3[nH]ccc3c2)nc1NC1CCC1] | A |
| 161b | 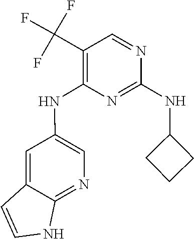 [SMILES: FC(F)(F)c1cnc(NC2CCC2)nc1Nc1cnc2[nH]ccc2c1] | A |
| 163a | 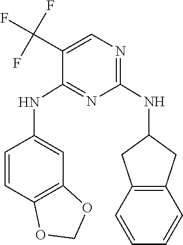 [SMILES: FC(F)(F)c1cnc(NC2Cc3ccccc3C2)nc1Nc1ccc2c(c1)OCO2] | C |
| 163b | 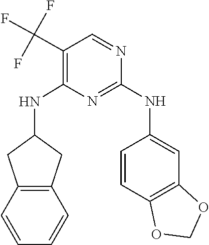 [SMILES: FC(F)(F)c1cnc(Nc2ccc3c(c2)OCO3)nc1NC1Cc2ccccc2C1] | C |
| 164a | 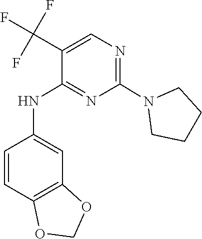 [SMILES: FC(F)(F)c1cnc(N2CCCC2)nc1Nc1ccc2c(c1)OCO2] | C |
| 164b | 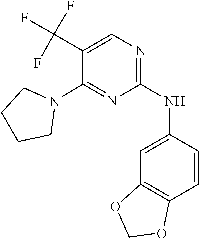 [SMILES: FC(F)(F)c1cnc(Nc2ccc3c(c2)OCO3)nc1N1CCCC1] | B |
| 165a | 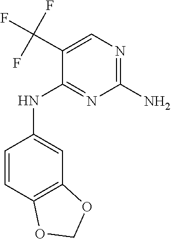 [SMILES: Nc1ncc(C(F)(F)F)c(Nc2ccc3c(c2)OCO3)n1] | B |
| 165b | 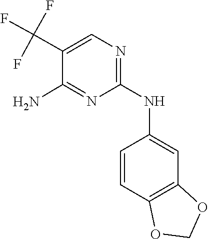 [SMILES: Nc1nc(Nc2ccc3c(c2)OCO3)ncc1C(F)(F)F] | B |
| 166a | 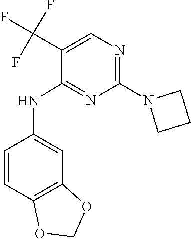 [SMILES: FC(F)(F)c1cnc(N2CCC2)nc1Nc1ccc2c(c1)OCO2] | C |
| 166b | 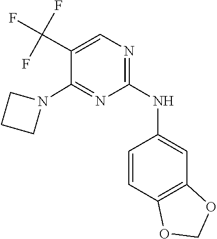 [SMILES: FC(F)(F)c1cnc(Nc2ccc3c(c2)OCO3)nc1N1CCC1] | A |
| 167a | 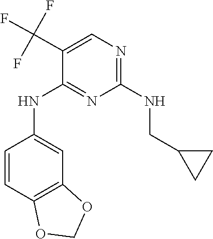 [SMILES: FC(F)(F)c1cnc(NCC2CC2)nc1Nc1ccc2c(c1)OCO2] | A |
| 167b | 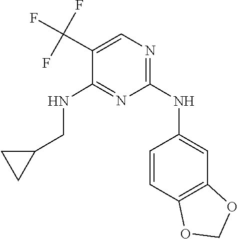 [SMILES: FC(F)(F)c1cnc(Nc2ccc3c(c2)OCO3)nc1NCC1CC1] | A |
| 168a | 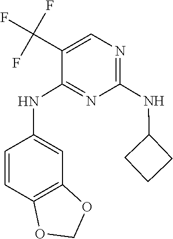 [SMILES: FC(F)(F)c1cnc(NC2CCC2)nc1Nc1ccc2c(c1)OCO2] | A |
| 168b | 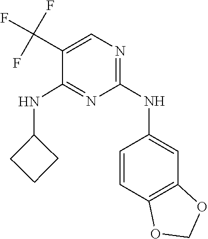 [SMILES: FC(F)(F)c1cnc(Nc2ccc3c(c2)OCO3)nc1NC1CCC1] | B |
| 168c | 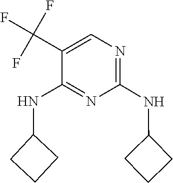 [SMILES: FC(F)(F)c1cnc(NC2CCC2)nc1NC1CCC1] | B |
| 169 | 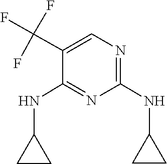 [SMILES: FC(F)(F)c1cnc(NC2CC2)nc1NC1CC1] | C |
| 170 | 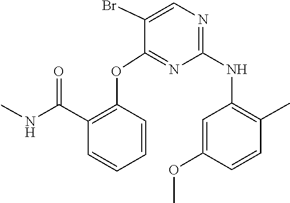 | C |
| 171a | 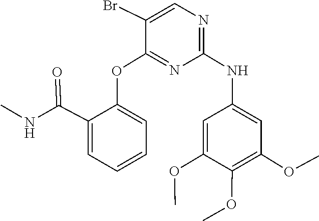 | A |
| 171b | 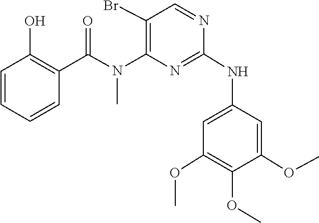 [SMILES: COc1cc(Nc2ncc(Br)c(N(C)C(=O)c3ccccc3O)n2)cc(OC)c1OC] | A |
| 172 | 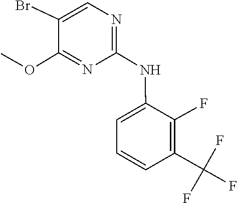 [SMILES: COc1nc(Nc2cccc(C(F)(F)F)c2F)ncc1Br] | B |
| 173 | 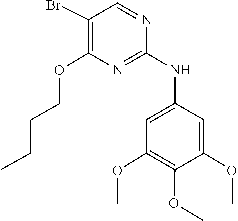 [SMILES: CCCCOc1nc(Nc2cc(OC)c(OC)c(OC)c2)ncc1Br] | A |
| 174 | 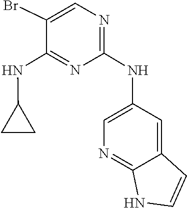 [SMILES: Brc1cnc(Nc2cnc3[nH]ccc3c2)nc1NC1CC1] | A |
| 175 | 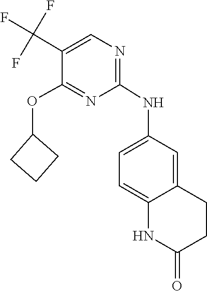 [SMILES: O=C1CCc2cc(Nc3ncc(C(F)(F)F)c(OC4CCC4)n3)ccc2N1] | A |
| 177 | 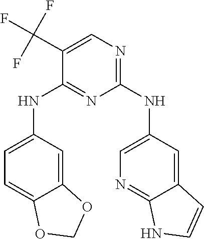 [SMILES: FC(F)(F)c1cnc(Nc2cnc3[nH]ccc3c2)nc1Nc1ccc2c(c1)OCO2] | A |
| 178a | 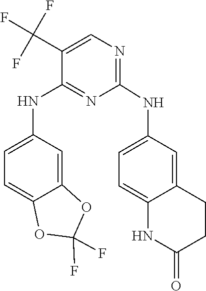 [SMILES: O=C1CCc2cc(Nc3ncc(C(F)(F)F)c(Nc4ccc5c(c4)OC(F)(F)O5)n3)ccc2N1] | A |
| 178b | 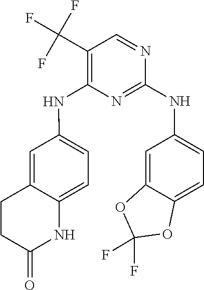 [SMILES: O=C1CCc2cc(Nc3nc(Nc4ccc5c(c4)OC(F)(F)O5)ncc3C(F)(F)F)ccc2N1] | A |
| 179 | 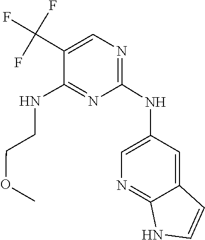 [SMILES: COCCNc1nc(Nc2cnc3[nH]ccc3c2)ncc1C(F)(F)F] | B |
| 180a | 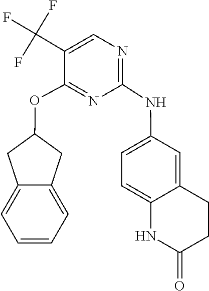 [SMILES: O=C1CCc2cc(Nc3ncc(C(F)(F)F)c(OC4Cc5ccccc5C4)n3)ccc2N1] | A |
| 180b | 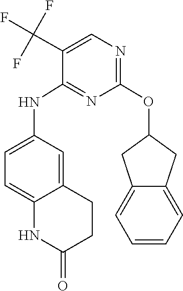 [SMILES: O=C1CCc2cc(Nc3nc(OC4Cc5ccccc5C4)ncc3C(F)(F)F)ccc2N1] | C |
| 181a | 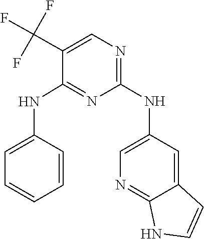 [SMILES: FC(F)(F)c1cnc(Nc2cnc3[nH]ccc3c2)nc1Nc1ccccc1] | A |
| 181b | 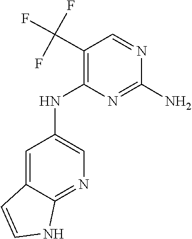 [SMILES: Nc1ncc(C(F)(F)F)c(Nc2cnc3[nH]ccc3c2)n1] | B |
| 182a | 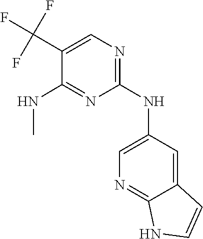 [SMILES: CNc1nc(Nc2cnc3[nH]ccc3c2)ncc1C(F)(F)F] | A |
| 182b | 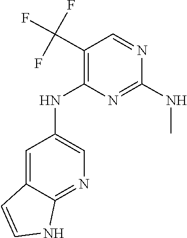 [SMILES: CNc1ncc(C(F)(F)F)c(Nc2cnc3[nH]ccc3c2)n1] | B |
| 183a | 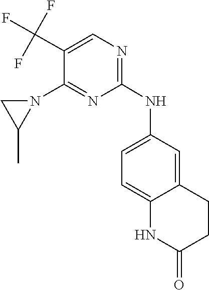 [SMILES: CC1CN1c1nc(Nc2ccc3c(c2)CCC(=O)N3)ncc1C(F)(F)F] | C |
| 183b | 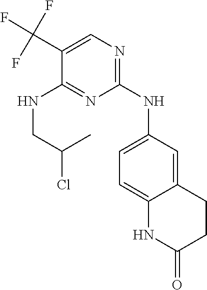 [SMILES: CC(Cl)CNc1nc(Nc2ccc3c(c2)CCC(=O)N3)ncc1C(F)(F)F] | A |
| 183c | 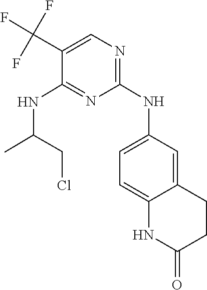 [SMILES: CC(CCl)Nc1nc(Nc2ccc3c(c2)CCC(=O)N3)ncc1C(F)(F)F] | C |
| 184a | 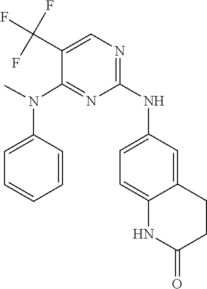 [SMILES: CN(c1ccccc1)c1nc(Nc2ccc3c(c2)CCC(=O)N3)ncc1C(F)(F)F] | B |
| 184b | 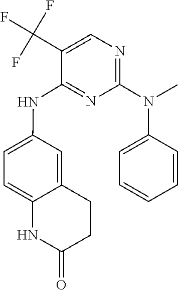 [SMILES: CN(c1ccccc1)c1ncc(C(F)(F)F)c(Nc2ccc3c(c2)CCC(=O)N3)n1] | A |
| 185 | 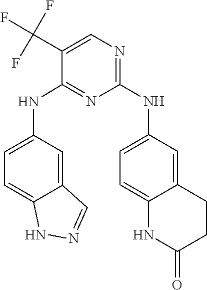 [SMILES: O=C1CCc2cc(Nc3ncc(C(F)(F)F)c(Nc4ccc5[nH]ncc5c4)n3)ccc2N1] | A |
| 186 | 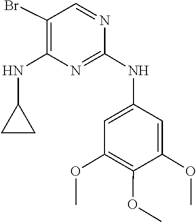 [SMILES: COc1cc(Nc2ncc(Br)c(NC3CC3)n2)cc(OC)c1OC] | A |
| 187 | 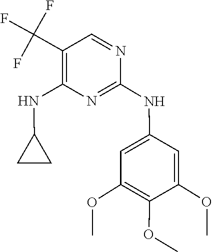 [SMILES: COc1cc(Nc2ncc(C(F)(F)F)c(NC3CC3)n2)cc(OC)c1OC] | A |
| 188 | 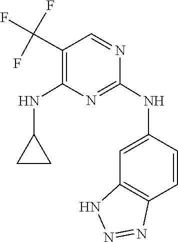 [SMILES: FC(F)(F)c1cnc(Nc2ccc3nn[nH]c3c2)nc1NC1CC1] | A |
| 189 | 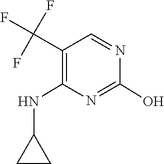 [SMILES: Oc1ncc(C(F)(F)F)c(NC2CC2)n1] | C |
| 190 | 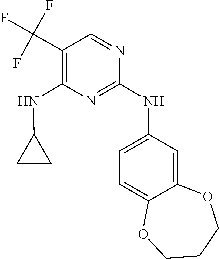 [SMILES: FC(F)(F)c1cnc(Nc2ccc3c(c2)OCCCO3)nc1NC1CC1] | A |
| 192 | 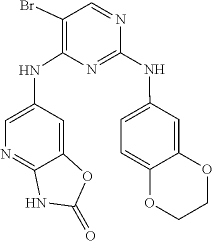 [SMILES: O=c1[nH]c2ncc(Nc3nc(Nc4ccc5c(c4)OCCO5)ncc3Br)cc2o1] | A |
| 193 | 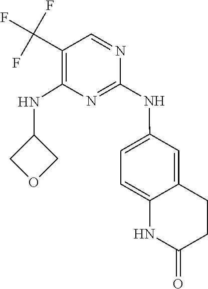 [SMILES: O=C1CCc2cc(Nc3ncc(C(F)(F)F)c(NC4COC4)n3)ccc2N1] | C |
| 194 | 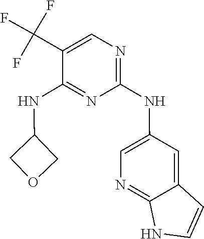 [SMILES: FC(F)(F)c1cnc(Nc2cnc3[nH]ccc3c2)nc1NC1COC1] | A |
| 195 | 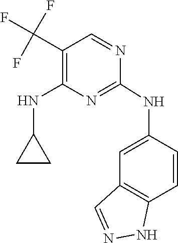 [SMILES: FC(F)(F)c1cnc(Nc2ccc3[nH]ncc3c2)nc1NC1CC1] | A |
| 196 | 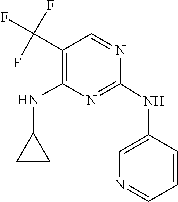 [SMILES: FC(F)(F)c1cnc(Nc2cccnc2)nc1NC1CC1] | C |
| 197 | 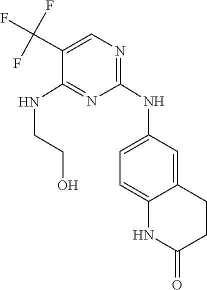 [SMILES: O=C1CCc2cc(Nc3ncc(C(F)(F)F)c(NCCO)n3)ccc2N1] | A |
| 198a | 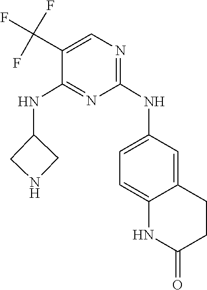 [SMILES: O=C1CCc2cc(Nc3ncc(C(F)(F)F)c(NC4CNC4)n3)ccc2N1] | B |
| 198b | 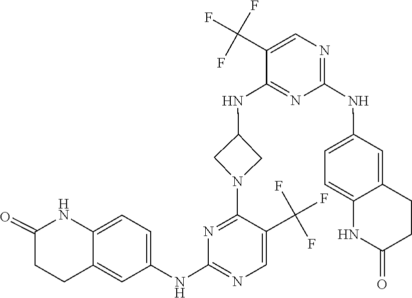 [SMILES: O=C1CCc2cc(Nc3ncc(C(F)(F)F)c(NC4CN(c5nc(Nc6ccc7c(c6)CCC(=O)N7)ncc5C(F)(F)F)C4)n3)ccc2N1] | C |
| 199 | 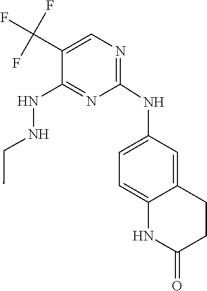 [SMILES: CCNNc1nc(Nc2ccc3c(c2)CCC(=O)N3)ncc1C(F)(F)F] | A |
| 200 | 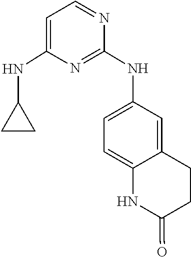 [SMILES: O=C1CCc2cc(Nc3nccc(NC4CC4)n3)ccc2N1] | C |
| 201 | 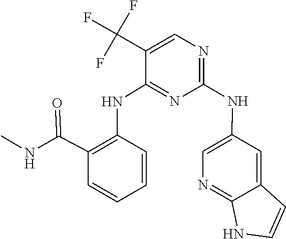 [SMILES: CNC(=O)c1ccccc1Nc1nc(Nc2cnc3[nH]ccc3c2)ncc1C(F)(F)F] | A |
| 202 | 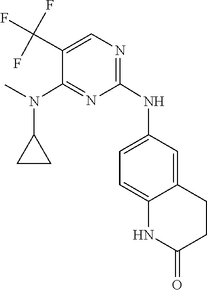 [SMILES: CN(c1nc(Nc2ccc3c(c2)CCC(=O)N3)ncc1C(F)(F)F)C1CC1] | A |
| 203 | 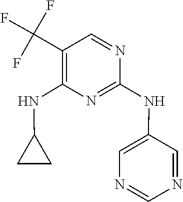 [SMILES: FC(F)(F)c1cnc(Nc2cncnc2)nc1NC1CC1] | A |
| 204 | 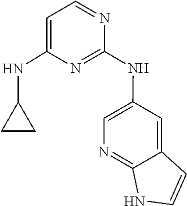 [SMILES: c1cc(NC2CC2)nc(Nc2cnc3[nH]ccc3c2)n1] | C |
| 205 | 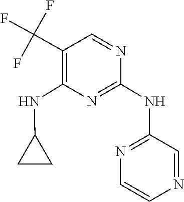 [SMILES: FC(F)(F)c1cnc(Nc2cnccn2)nc1NC1CC1] | C |
| 206 | 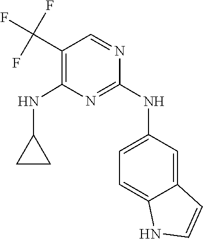 [SMILES: FC(F)(F)c1cnc(Nc2ccc3[nH]ccc3c2)nc1NC1CC1] | A |
| 207 | 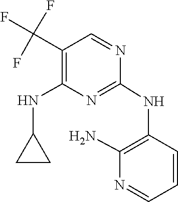 [SMILES: Nc1ncccc1Nc1ncc(C(F)(F)F)c(NC2CC2)n1] | C |
| 208a | 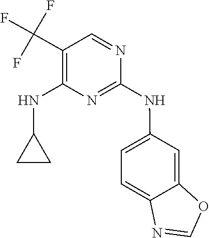 [SMILES: FC(F)(F)c1cnc(Nc2ccc3ncoc3c2)nc1NC1CC1] | A |
| 208b | 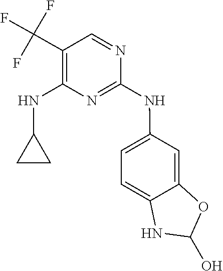 [SMILES: OC1Nc2ccc(Nc3ncc(C(F)(F)F)c(NC4CC4)n3)cc2O1] | A |
| 209 | 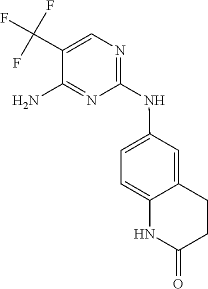 [SMILES: Nc1nc(Nc2ccc3c(c2)CCC(=O)N3)ncc1C(F)(F)F] | B |
| 210 | 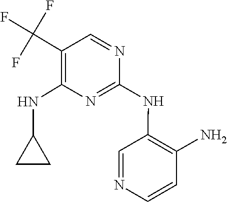 [SMILES: Nc1ccncc1Nc1ncc(C(F)(F)F)c(NC2CC2)n1] | B |
| 211 | 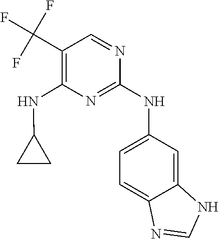 [SMILES: FC(F)(F)c1cnc(Nc2ccc3nc[nH]c3c2)nc1NC1CC1] | A |
| 212a | 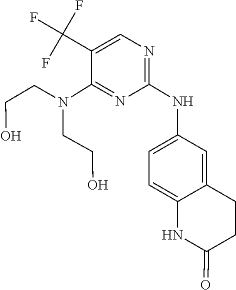 [SMILES: O=C1CCc2cc(Nc3ncc(C(F)(F)F)c(N(CCO)CCO)n3)ccc2N1] | C |
| 212b | 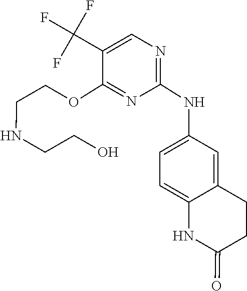 [SMILES: O=C1CCc2cc(Nc3ncc(C(F)(F)F)c(OCCNCCO)n3)ccc2N1] | B |
| 213 | 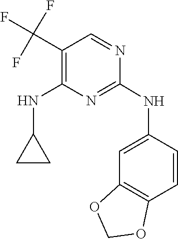 [SMILES: FC(F)(F)c1cnc(Nc2ccc3c(c2)OCO3)nc1NC1CC1] | A |
| 214a | 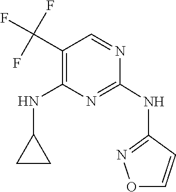 [SMILES: FC(F)(F)c1cnc(Nc2ccon2)nc1NC1CC1] | B |
| 214b | 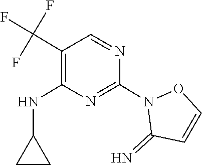 [SMILES: N=c1ccon1-c1ncc(C(F)(F)F)c(NC2CC2)n1] | C |
| 215 | 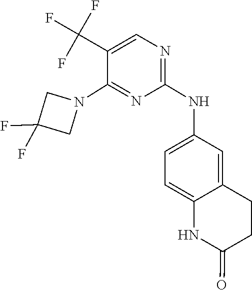 [SMILES: O=C1CCc2cc(Nc3ncc(C(F)(F)F)c(N4CC(F)(F)C4)n3)ccc2N1] | A |
| 216 | 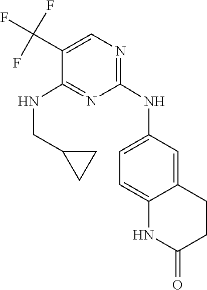 [SMILES: O=C1CCc2cc(Nc3ncc(C(F)(F)F)c(NCC4CC4)n3)ccc2N1] | A |
| 217 | 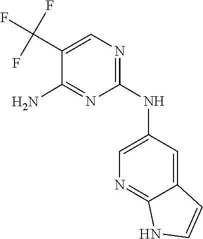 [SMILES: Nc1nc(Nc2cnc3[nH]ccc3c2)ncc1C(F)(F)F] | C |
| 218 | 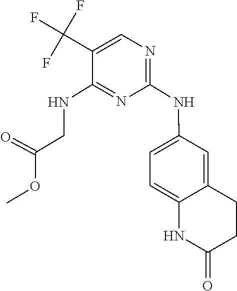 [SMILES: COC(=O)CNc1nc(Nc2ccc3c(c2)CCC(=O)N3)ncc1C(F)(F)F] | C |
| 219 | 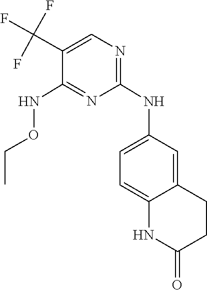 [SMILES: CCONc1nc(Nc2ccc3c(c2)CCC(=O)N3)ncc1C(F)(F)F] | C |
| 220a | 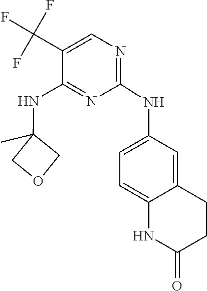 [SMILES: CC1(Nc2nc(Nc3ccc4c(c3)CCC(=O)N4)ncc2C(F)(F)F)COC1] | C |
| 220b | 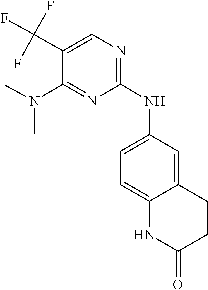 [SMILES: CN(C)c1nc(Nc2ccc3c(c2)CCC(=O)N3)ncc1C(F)(F)F] | A |
| 220c | 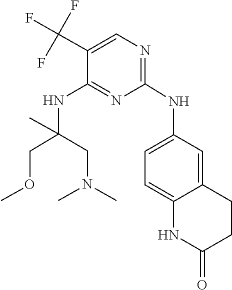 [SMILES: COCC(C)(CN(C)C)Nc1nc(Nc2ccc3c(c2)CCC(=O)N3)ncc1C(F)(F)F] | C |
| 221a | 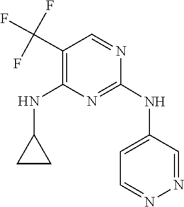 [SMILES: FC(F)(F)c1cnc(Nc2ccnnc2)nc1NC1CC1] | A |
| 222 | 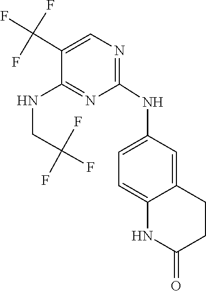 [SMILES: O=C1CCc2cc(Nc3ncc(C(F)(F)F)c(NCC(F)(F)F)n3)ccc2N1] | A |
| 223 | 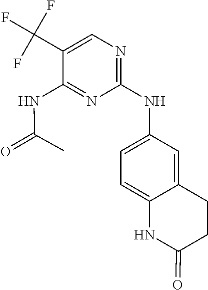 [SMILES: CC(=O)Nc1nc(Nc2ccc3c(c2)CCC(=O)N3)ncc1C(F)(F)F] | C |
| 224 | 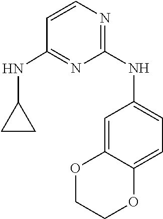 [SMILES: c1cc(NC2CC2)nc(Nc2ccc3c(c2)OCCO3)n1] | C |
| 225 | 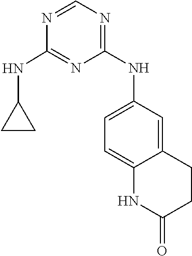 [SMILES: O=C1CCc2cc(Nc3ncnc(NC4CC4)n3)ccc2N1] | C |
| 226 | 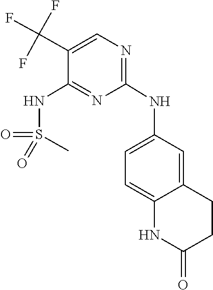 [SMILES: CS(=O)(=O)Nc1nc(Nc2ccc3c(c2)CCC(=O)N3)ncc1C(F)(F)F] | C |
| 227 | 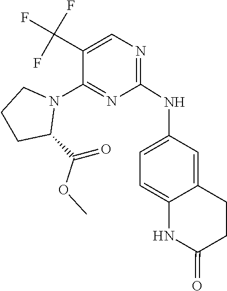 [SMILES: COC(=O)[C@@H]1CCCN1c1nc(Nc2ccc3c(c2)CCC(=O)N3)ncc1C(F)(F)F] | B |
| 228 | 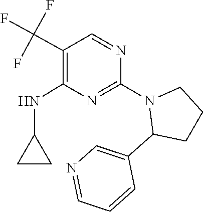 [SMILES: FC(F)(F)c1cnc(N2CCCC2c2cccnc2)nc1NC1CC1] | C |
| 229 | 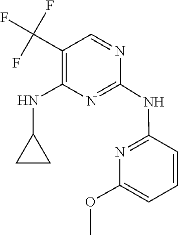 [SMILES: COc1cccc(Nc2ncc(C(F)(F)F)c(NC3CC3)n2)n1] | A |
| 230 | 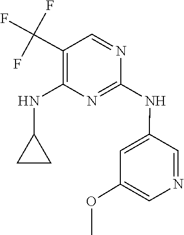 [SMILES: COc1cncc(Nc2ncc(C(F)(F)F)c(NC3CC3)n2)c1] | A |
| 231a | 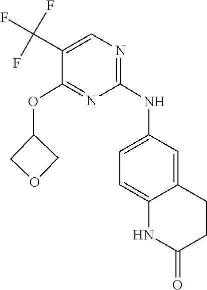 [SMILES: O=C1CCc2cc(Nc3ncc(C(F)(F)F)c(OC4COC4)n3)ccc2N1] | A |
| 231b | 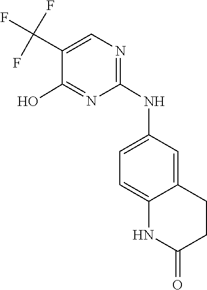 [SMILES: O=C1CCc2cc(Nc3ncc(C(F)(F)F)c(O)n3)ccc2N1] | C |
| 232 | 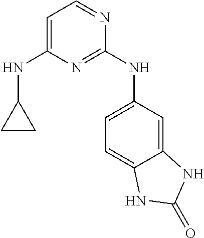 [SMILES: O=c1[nH]c2ccc(Nc3nccc(NC4CC4)n3)cc2[nH]1] | C |
| 233 | 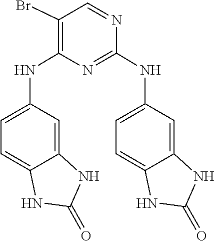 [SMILES: O=c1[nH]c2ccc(Nc3ncc(Br)c(Nc4ccc5[nH]c(=O)[nH]c5c4)n3)cc2[nH]1] | A |
| 234a | 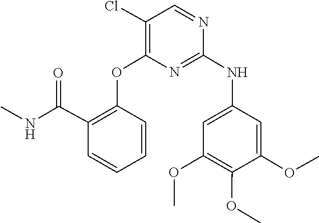 | C |
| 234b | 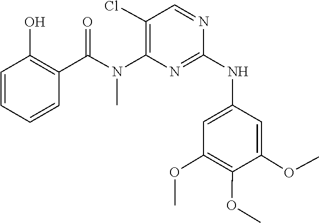 [SMILES: COc1cc(Nc2ncc(Cl)c(N(C)C(=O)c3ccccc3O)n2)cc(OC)c1OC] | B |
| 235 | 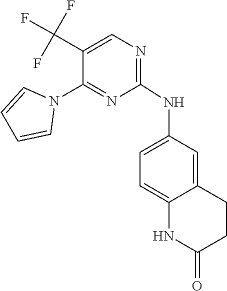 [SMILES: O=C1CCc2cc(Nc3ncc(C(F)(F)F)c(-n4cccc4)n3)ccc2N1] | C |
| 236 | 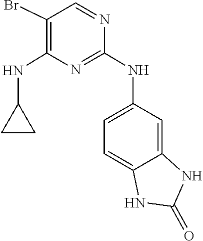 [SMILES: O=c1[nH]c2ccc(Nc3ncc(Br)c(NC4CC4)n3)cc2[nH]1] | A |
| 237 | 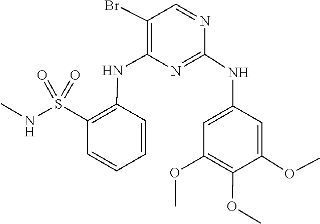 | B |
| 239 | 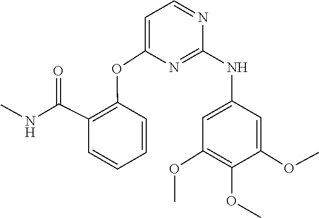 | C |
| 240 | 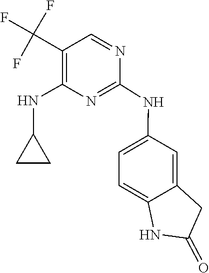 [SMILES: O=C1Cc2cc(Nc3ncc(C(F)(F)F)c(NC4CC4)n3)ccc2N1] | A |
| 242 | 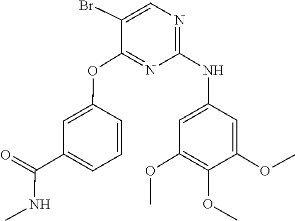 | B |
| 243 | 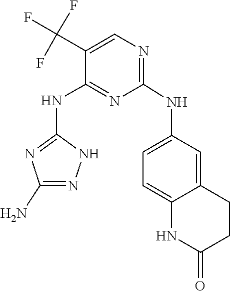 [SMILES: Nc1n[nH]c(Nc2nc(Nc3ccc4c(c3)CCC(=O)N4)ncc2C(F)(F)F)n1] | B |
| 245 | 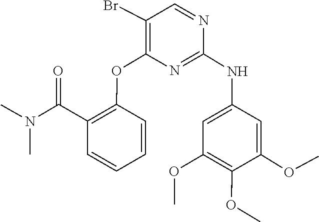 | A |
| 246 | 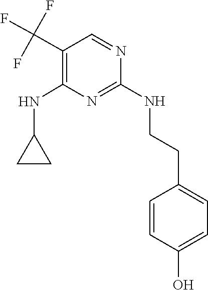 [SMILES: Oc1ccc(CCNc2ncc(C(F)(F)F)c(NC3CC3)n2)cc1] | B |
| 247 | 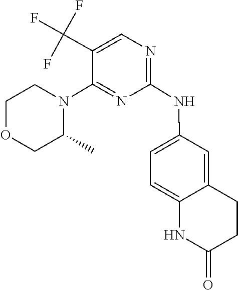 [SMILES: C[C@@H]1COCCN1c1nc(Nc2ccc3c(c2)CCC(=O)N3)ncc1C(F)(F)F] | A |
| 249 | 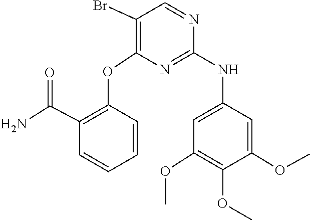 [SMILES: COc1cc(Nc2ncc(Br)c(Oc3ccccc3C(N)=O)n2)cc(OC)c1OC] | A |
| 250 | 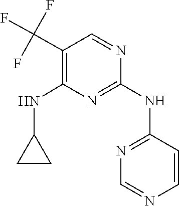 [SMILES: FC(F)(F)c1cnc(Nc2ccncn2)nc1NC1CC1] | C |
| 252 | 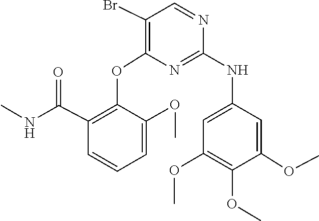 | A |
| 254a | 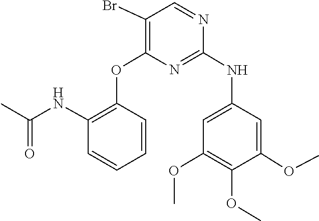 [SMILES: COc1cc(Nc2ncc(Br)c(Oc3ccccc3NC(C)=O)n2)cc(OC)c1OC] | A |
| 254b | 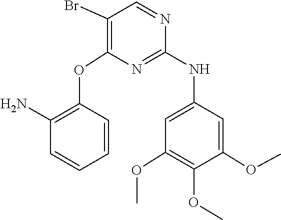 [SMILES: COc1cc(Nc2ncc(Br)c(Oc3ccccc3N)n2)cc(OC)c1OC] | A |
| 255 | 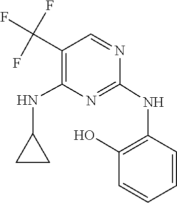 [SMILES: Oc1ccccc1Nc1ncc(C(F)(F)F)c(NC2CC2)n1] | A |
| 257 | 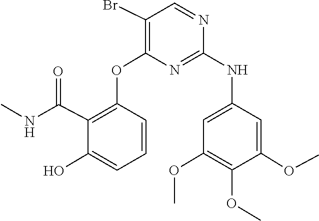 | A |
| 258 | 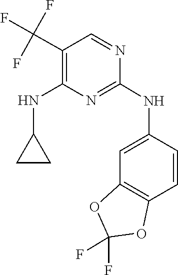 [SMILES: FC1(F)Oc2ccc(Nc3ncc(C(F)(F)F)c(NC4CC4)n3)cc2O1] | C |
| 260 | 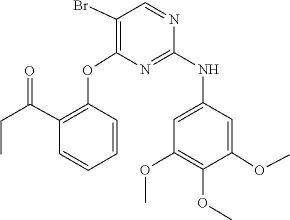 [SMILES: CCC(=O)c1ccccc1Oc1nc(Nc2cc(OC)c(OC)c(OC)c2)ncc1Br] | A |
| 261 | 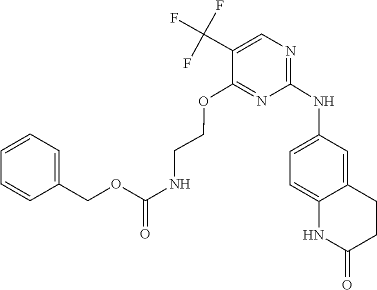 [SMILES: O=C1CCc2cc(Nc3ncc(C(F)(F)F)c(OCCNC(=O)OCc4ccccc4)n3)ccc2N1] | C |
| 262 | 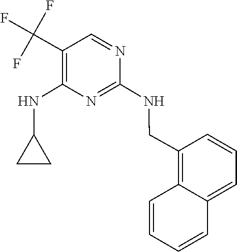 [SMILES: FC(F)(F)c1cnc(NCc2cccc3ccccc23)nc1NC1CC1] | C |
| 263 | 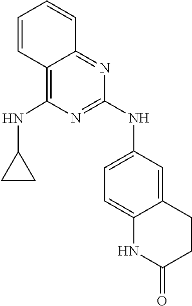 [SMILES: O=C1CCc2cc(Nc3nc(NC4CC4)c4ccccc4n3)ccc2N1] | C |
| 264 | 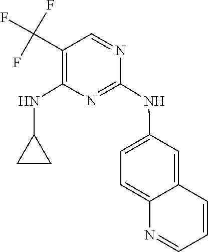 [SMILES: FC(F)(F)c1cnc(Nc2ccc3ncccc3c2)nc1NC1CC1] | A |
| 266 | 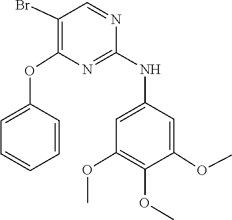 [SMILES: COc1cc(Nc2ncc(Br)c(Oc3ccccc3)n2)cc(OC)c1OC] | A |
| 268 | 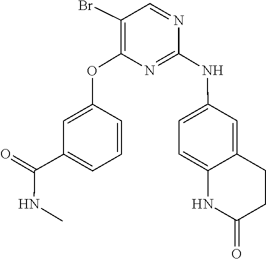 [SMILES: CNC(=O)c1cccc(Oc2nc(Nc3ccc4c(c3)CCC(=O)N4)ncc2Br)c1] | B |
| 270 | 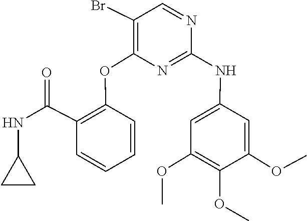 [SMILES: COc1cc(Nc2ncc(Br)c(Oc3ccccc3C(=O)NC3CC3)n2)cc(OC)c1OC] | B |
| 271 | 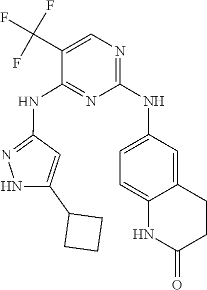 [SMILES: O=C1CCc2cc(Nc3ncc(C(F)(F)F)c(Nc4cc(C5CCC5)[nH]n4)n3)ccc2N1] | A |
| 274 | 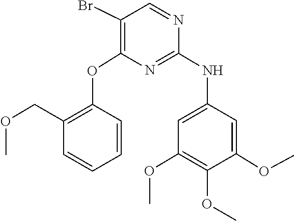 [SMILES: COCc1ccccc1Oc1nc(Nc2cc(OC)c(OC)c(OC)c2)ncc1Br] | A |
| 275 | 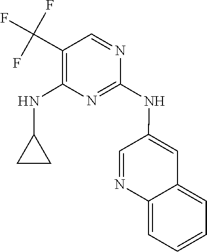 [SMILES: FC(F)(F)c1cnc(Nc2cnc3ccccc3c2)nc1NC1CC1] | A |
| 277 | 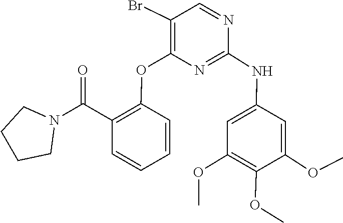 [SMILES: COc1cc(Nc2ncc(Br)c(Oc3ccccc3C(=O)N3CCCC3)n2)cc(OC)c1OC] | A |
| 278 | 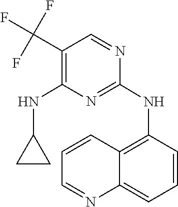 [SMILES: FC(F)(F)c1cnc(Nc2cccc3ncccc23)nc1NC1CC1] | C |
| 281 | 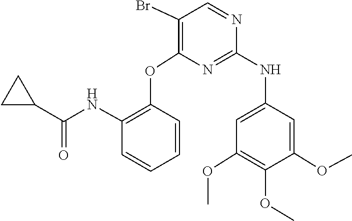 [SMILES: COc1cc(Nc2ncc(Br)c(Oc3ccccc3NC(=O)C3CC3)n2)cc(OC)c1OC] | A |
| 283 | 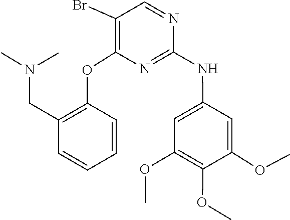 | C |
| 286 | 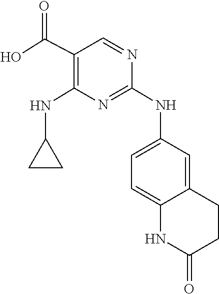 [SMILES: O=C1CCc2cc(Nc3ncc(C(=O)O)c(NC4CC4)n3)ccc2N1] | C |
| 287 | 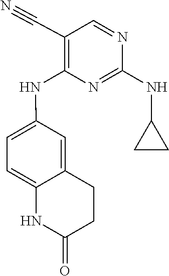 [SMILES: N#Cc1cnc(NC2CC2)nc1Nc1ccc2c(c1)CCC(=O)N2] | C |
| 288 | 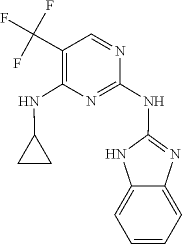 [SMILES: FC(F)(F)c1cnc(Nc2nc3ccccc3[nH]2)nc1NC1CC1] | C |
| 290 | 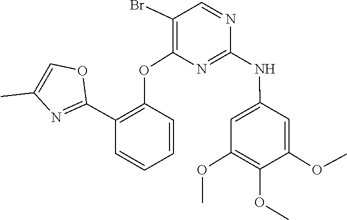 | C |
| 291b | 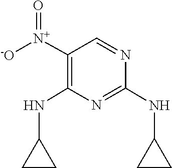 | C |
| 293 | 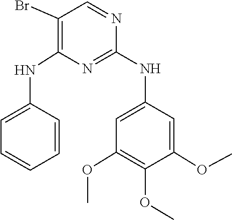 [SMILES: COc1cc(Nc2ncc(Br)c(Nc3ccccc3)n2)cc(OC)c1OC] | A |
| 294 | 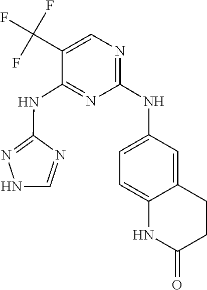 [SMILES: O=C1CCc2cc(Nc3ncc(C(F)(F)F)c(Nc4nc[nH]n4)n3)ccc2N1] | C |
| 296 | 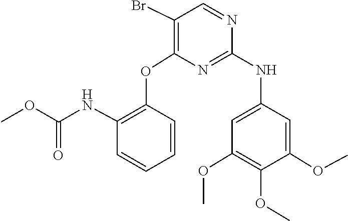 | A |
| 297 | 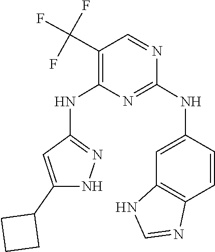 [SMILES: FC(F)(F)c1cnc(Nc2ccc3nc[nH]c3c2)nc1Nc1cc(C2CCC2)[nH]n1] | A |
| 299 | 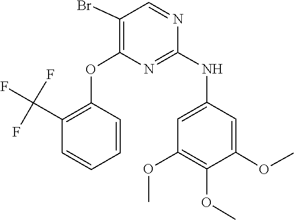 [SMILES: COc1cc(Nc2ncc(Br)c(Oc3ccccc3C(F)(F)F)n2)cc(OC)c1OC] | A |
| 300a | 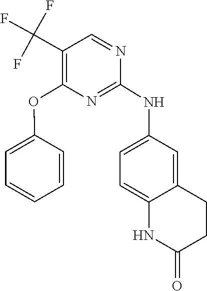 [SMILES: O=C1CCc2cc(Nc3ncc(C(F)(F)F)c(Oc4ccccc4)n3)ccc2N1] | C |
| 300b | 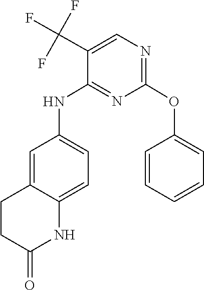 [SMILES: O=C1CCc2cc(Nc3nc(Oc4ccccc4)ncc3C(F)(F)F)ccc2N1] | C |
| 301 | 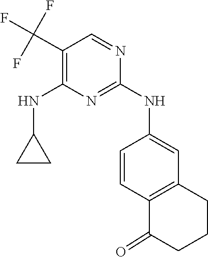 [SMILES: O=C1CCCc2cc(Nc3ncc(C(F)(F)F)c(NC4CC4)n3)ccc21] | A |
| 303 | 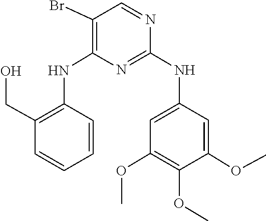 [SMILES: COc1cc(Nc2ncc(Br)c(Nc3ccccc3CO)n2)cc(OC)c1OC] | A |
| 304a | 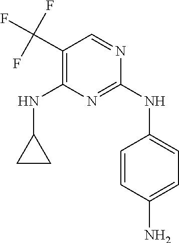 [SMILES: Nc1ccc(Nc2ncc(C(F)(F)F)c(NC3CC3)n2)cc1] | A |
| 304b | 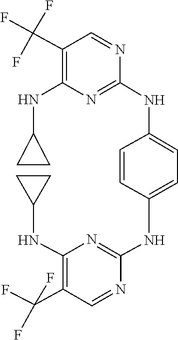 [SMILES: FC(F)(F)c1cnc(Nc2ccc(Nc3ncc(C(F)(F)F)c(NC4CC4)n3)cc2)nc1NC1CC1] | C |
| 305 | 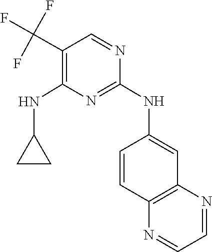 [SMILES: FC(F)(F)c1cnc(Nc2ccc3nccnc3c2)nc1NC1CC1] | A |
| 307 | 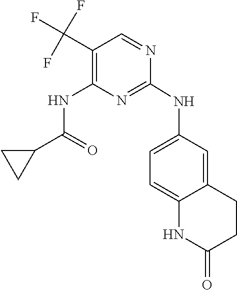 [SMILES: O=C1CCc2cc(Nc3ncc(C(F)(F)F)c(NC(=O)C4CC4)n3)ccc2N1] | C |
| 308 | 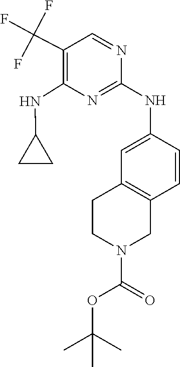 [SMILES: CC(C)(C)OC(=O)N1CCc2cc(Nc3ncc(C(F)(F)F)c(NC4CC4)n3)ccc2C1] | A |
| 309 | 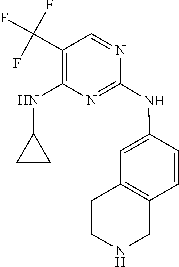 [SMILES: FC(F)(F)c1cnc(Nc2ccc3c(c2)CCNC3)nc1NC1CC1] | A |
| 310 | 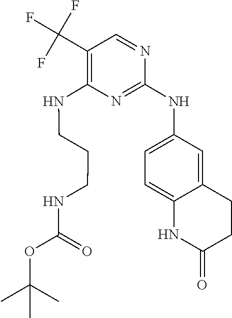 [SMILES: CC(C)(C)OC(=O)NCCCNc1nc(Nc2ccc3c(c2)CCC(=O)N3)ncc1C(F)(F)F] | A |
| 311 | 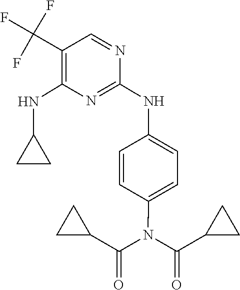 [SMILES: O=C(C1CC1)N(C(=O)C1CC1)c1ccc(Nc2ncc(C(F)(F)F)c(NC3CC3)n2)cc1] | A |
| 312 | 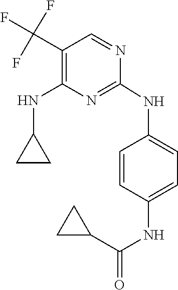 [SMILES: O=C(Nc1ccc(Nc2ncc(C(F)(F)F)c(NC3CC3)n2)cc1)C1CC1] | A |
| 313 | 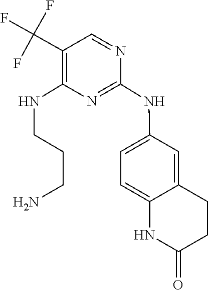 [SMILES: NCCCNc1nc(Nc2ccc3c(c2)CCC(=O)N3)ncc1C(F)(F)F] | A |
| 314 | 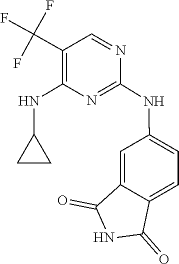 [SMILES: O=C1NC(=O)c2cc(Nc3ncc(C(F)(F)F)c(NC4CC4)n3)ccc21] | A |
| 316 | 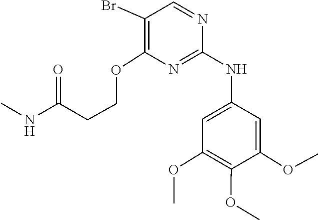 | A |
| 317 | 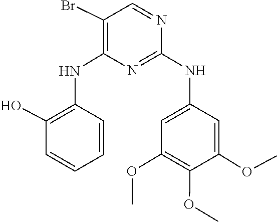 [SMILES: COc1cc(Nc2ncc(Br)c(Nc3ccccc3O)n2)cc(OC)c1OC] | A |
| 318a | 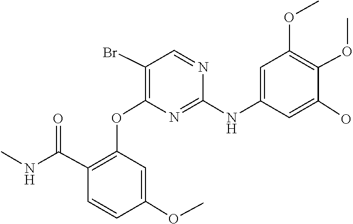 | C |
| 318b | 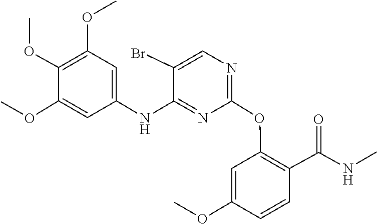 | C |
| 320 | 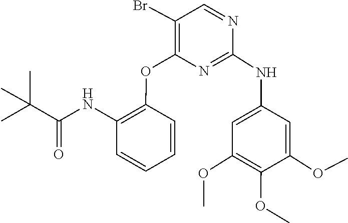 [SMILES: COc1cc(Nc2ncc(Br)c(Oc3ccccc3NC(=O)C(C)(C)C)n2)cc(OC)c1OC] | C |
| 321a | 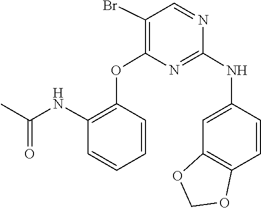 [SMILES: CC(=O)Nc1ccccc1Oc1nc(Nc2ccc3c(c2)OCO3)ncc1Br] | C |
| 321b | 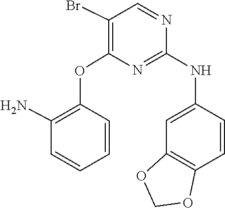 [SMILES: Nc1ccccc1Oc1nc(Nc2ccc3c(c2)OCO3)ncc1Br] | A |
| 323 | 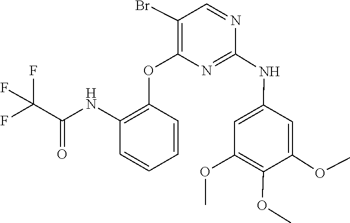 [SMILES: COc1cc(Nc2ncc(Br)c(Oc3ccccc3NC(=O)C(F)(F)F)n2)cc(OC)c1OC] | A |
| 324a | 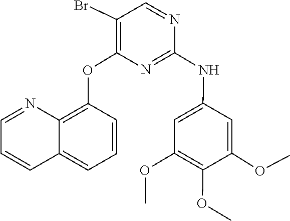 [SMILES: COc1cc(Nc2ncc(Br)c(Oc3cccc4cccnc34)n2)cc(OC)c1OC] | C |
| 324b | 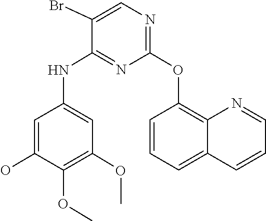 | A |
| 325 | 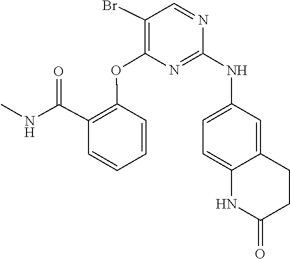 [SMILES: CNC(=O)c1ccccc1Oc1nc(Nc2ccc3c(c2)CCC(=O)N3)ncc1Br] | C |
| 326 | 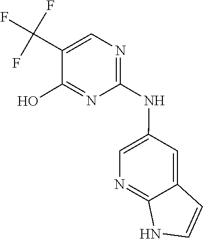 [SMILES: Oc1nc(Nc2cnc3[nH]ccc3c2)ncc1C(F)(F)F] | C |
| 327 | 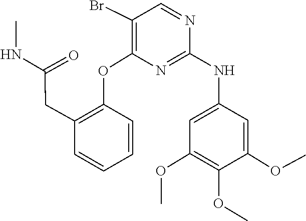 | C |
| 328 | 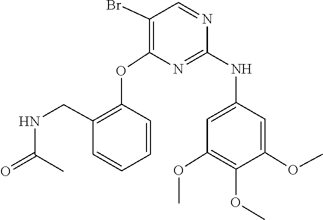 [SMILES: COc1cc(Nc2ncc(Br)c(Oc3ccccc3CNC(C)=O)n2)cc(OC)c1OC] | C |
| 329a | 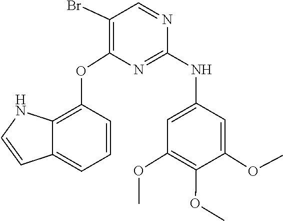 [SMILES: COc1cc(Nc2ncc(Br)c(Oc3cccc4cc[nH]c34)n2)cc(OC)c1OC] | C |
| 329b | 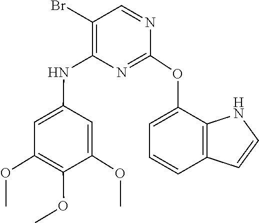 [SMILES: COc1cc(Nc2nc(Oc3cccc4cc[nH]c34)ncc2Br)cc(OC)c1OC] | C |
| 330a | 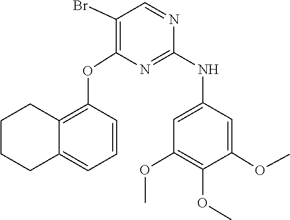 [SMILES: COc1cc(Nc2ncc(Br)c(Oc3cccc4c3CCCC4)n2)cc(OC)c1OC] | C |
| 330b | 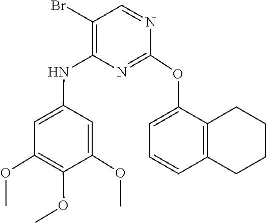 [SMILES: COc1cc(Nc2nc(Oc3cccc4c3CCCC4)ncc2Br)cc(OC)c1OC] | C |
| 331a | 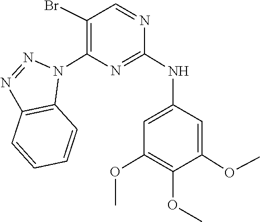 [SMILES: COc1cc(Nc2ncc(Br)c(-n3nnc4ccccc43)n2)cc(OC)c1OC] | C |
| 331b | 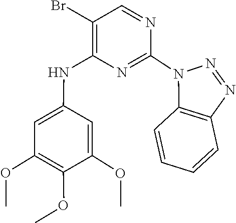 [SMILES: COc1cc(Nc2nc(-n3nnc4ccccc43)ncc2Br)cc(OC)c1OC] | B |
| 332 | 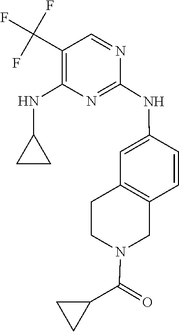 [SMILES: O=C(C1CC1)N1CCc2cc(Nc3ncc(C(F)(F)F)c(NC4CC4)n3)ccc2C1] | A |
| 333 | 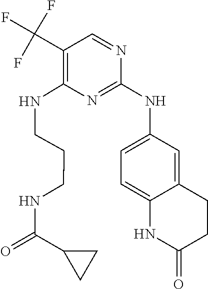 [SMILES: O=C1CCc2cc(Nc3ncc(C(F)(F)F)c(NCCCNC(=O)C4CC4)n3)ccc2N1] | A |
| 334 | 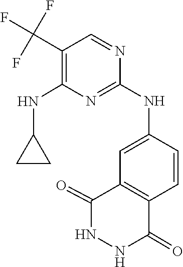 [SMILES: O=c1[nH][nH]c(=O)c2cc(Nc3ncc(C(F)(F)F)c(NC4CC4)n3)ccc12] | A |
| 335a | 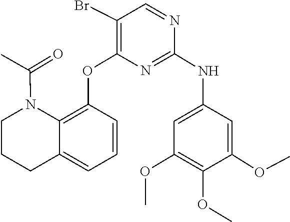 [SMILES: COc1cc(Nc2ncc(Br)c(Oc3cccc4c3N(C(C)=O)CCC4)n2)cc(OC)c1OC] | C |
| 335b | 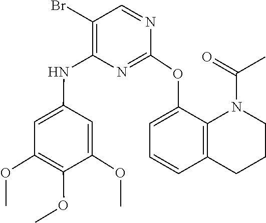 [SMILES: COc1cc(Nc2nc(Oc3cccc4c3N(C(C)=O)CCC4)ncc2Br)cc(OC)c1OC] | A |
| 336 | 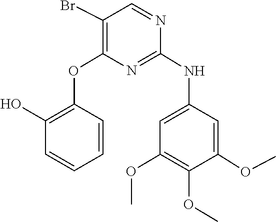 [SMILES: COc1cc(Nc2ncc(Br)c(Oc3ccccc3O)n2)cc(OC)c1OC] | C |
| 338 | 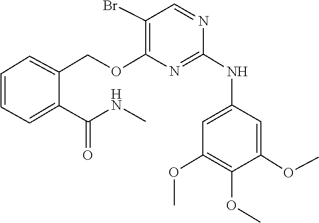 [SMILES: CNC(=O)c1ccccc1COc1nc(Nc2cc(OC)c(OC)c(OC)c2)ncc1Br] | C |
| 343 | 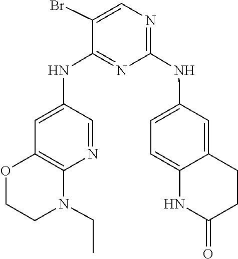 [SMILES: CCN1CCOc2cc(Nc3nc(Nc4ccc5c(c4)CCC(=O)N5)ncc3Br)cnc21] | A |
| 344 | 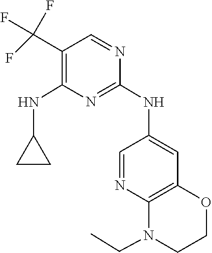 [SMILES: CCN1CCOc2cc(Nc3ncc(C(F)(F)F)c(NC4CC4)n3)cnc21] | A |
| 345 | 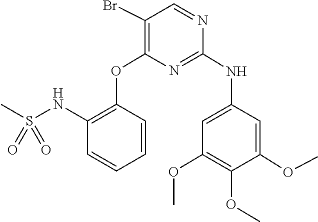 | C |
| 346 | 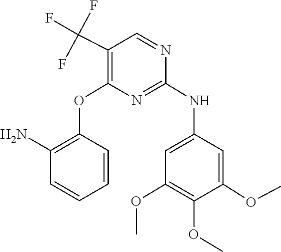 [SMILES: COc1cc(Nc2ncc(C(F)(F)F)c(Oc3ccccc3N)n2)cc(OC)c1OC] | A |
| 347 | 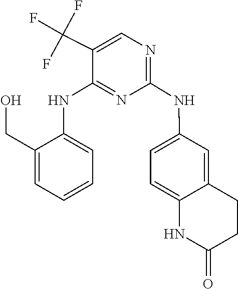 [SMILES: O=C1CCc2cc(Nc3ncc(C(F)(F)F)c(Nc4ccccc4CO)n3)ccc2N1] | A |
| 348 | 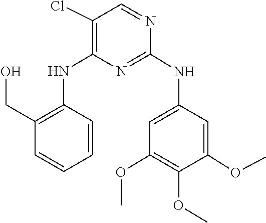 [SMILES: COc1cc(Nc2ncc(Cl)c(Nc3ccccc3CO)n2)cc(OC)c1OC] | C |
| 349 | 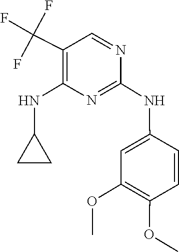 [SMILES: COc1ccc(Nc2ncc(C(F)(F)F)c(NC3CC3)n2)cc1OC] | A |
| 350 | 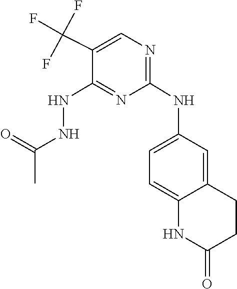 [SMILES: CC(=O)NNc1nc(Nc2ccc3c(c2)CCC(=O)N3)ncc1C(F)(F)F] | C |
| 351 | 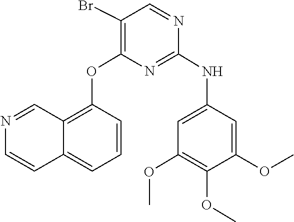 [SMILES: COc1cc(Nc2ncc(Br)c(Oc3cccc4ccncc34)n2)cc(OC)c1OC] | C |
| 352 | 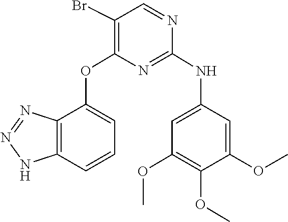 [SMILES: COc1cc(Nc2ncc(Br)c(Oc3cccc4[nH]nnc34)n2)cc(OC)c1OC] | C |
| 353 | 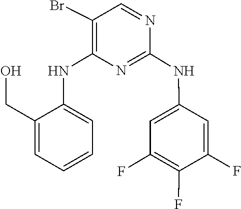 [SMILES: OCc1ccccc1Nc1nc(Nc2cc(F)c(F)c(F)c2)ncc1Br] | C |
| 354 | 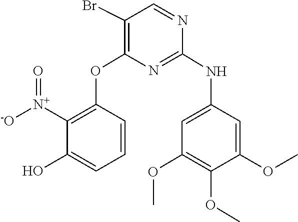 | C |
| 355 | 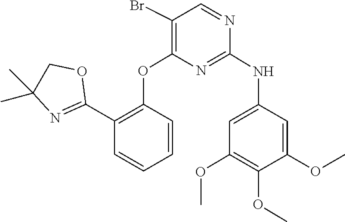 | C |
| 356 | 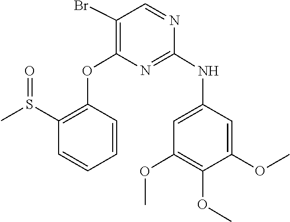 | C |
| 357 | 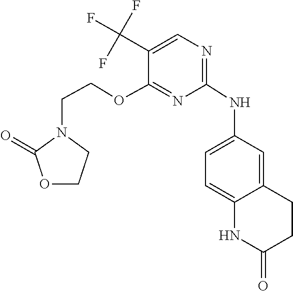 [SMILES: O=C1CCc2cc(Nc3ncc(C(F)(F)F)c(OCCN4CCOC4=O)n3)ccc2N1] | B |
| 358 | 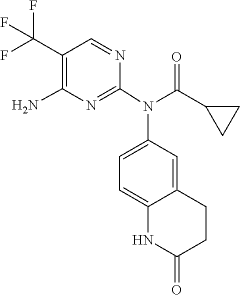 [SMILES: Nc1nc(N(C(=O)C2CC2)c2ccc3c(c2)CCC(=O)N3)ncc1C(F)(F)F] | C |

Exemplary compounds are illustrated in a non-limiting manner below:

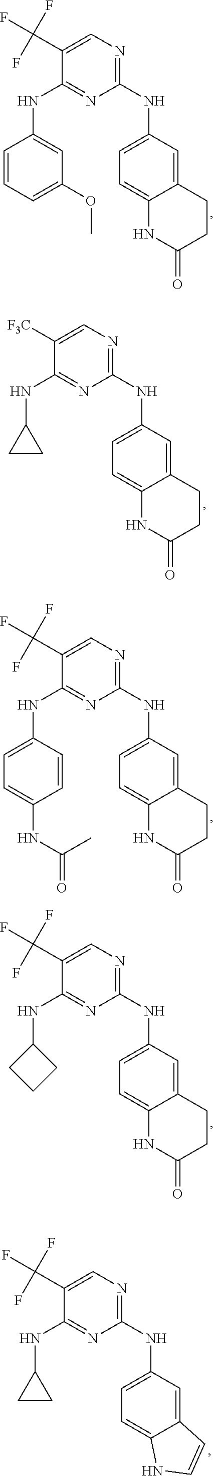

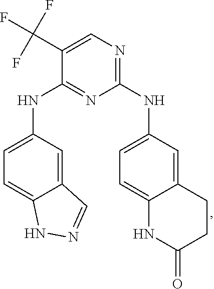 [SMILES: O=C1CCc2cc(Nc3ncc(C(F)(F)F)c(Nc4ccc5[nH]ncc5c4)n3)ccc2N1]

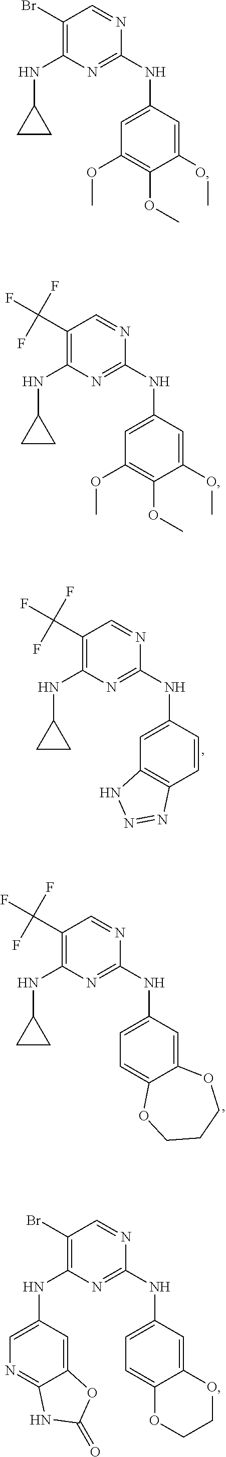 [SMILES: COc1cc(Nc2ncc(Br)c(NC3CC3)n2)cc(OC)c1OC ; COc1cc(Nc2ncc(C(F)(F)F)c(NC3CC3)n2)cc(OC)c1OC ; FC(F)(F)c1cnc(Nc2ccc3nn[nH]c3c2)nc1NC1CC1 ; FC(F)(F)c1cnc(Nc2ccc3c(c2)OCCCO3)nc1NC1CC1 ; O=c1[nH]c2ncc(Nc3nc(Nc4ccc5c(c4)OCCO5)ncc3Br)cc2o1]

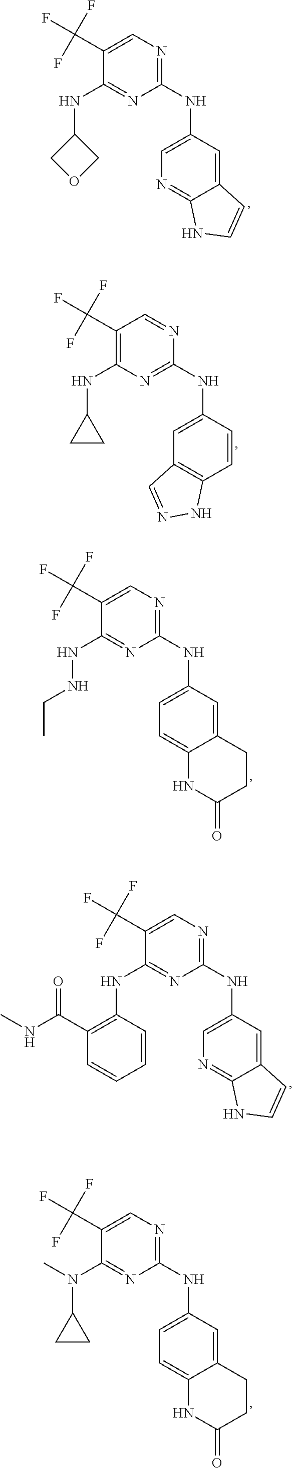 [SMILES: FC(F)(F)c1cnc(Nc2cnc3[nH]ccc3c2)nc1NC1COC1 ; FC(F)(F)c1cnc(Nc2ccc3[nH]ncc3c2)nc1NC1CC1 ; CCNNc1nc(Nc2ccc3c(c2)CCC(=O)N3)ncc1C(F)(F)F ; CNC(=O)c1ccccc1Nc1nc(Nc2cnc3[nH]ccc3c2)ncc1C(F)(F)F ; CN(c1nc(Nc2ccc3c(c2)CCC(=O)N3)ncc1C(F)(F)F)C1CC1]

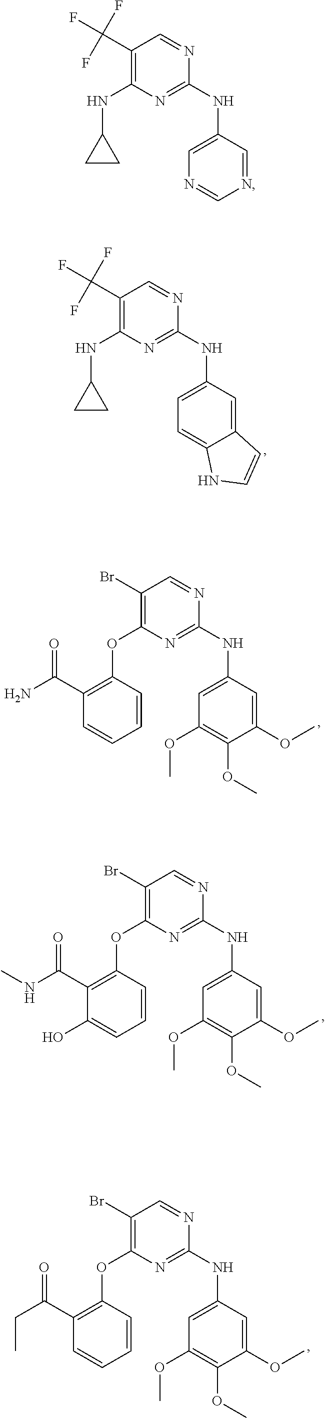

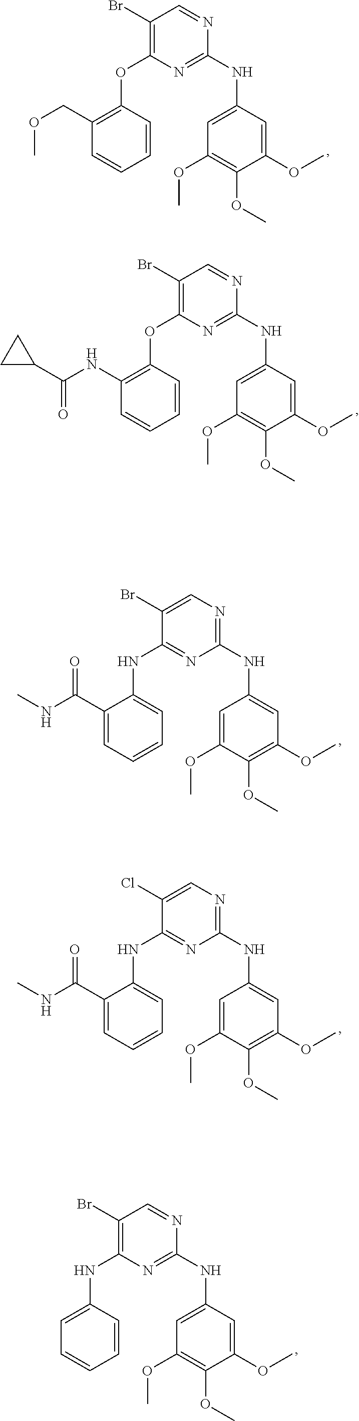

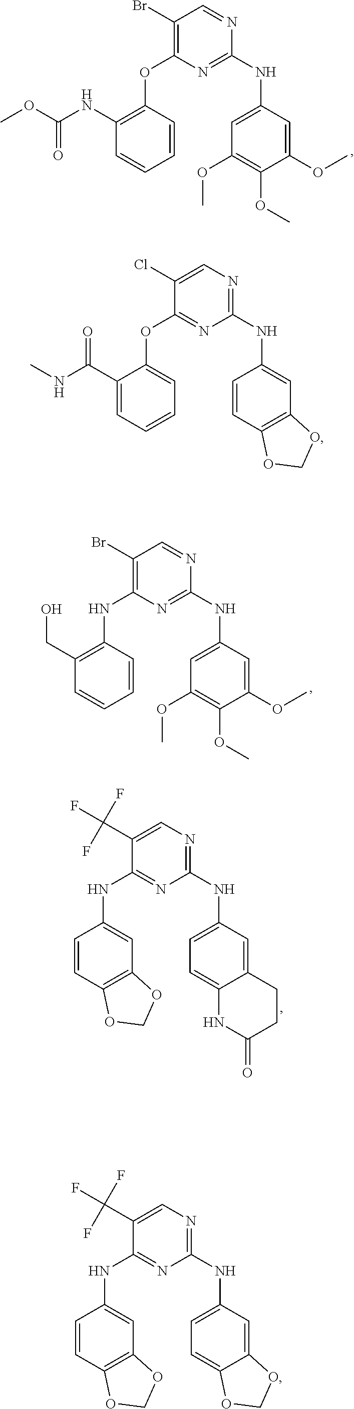

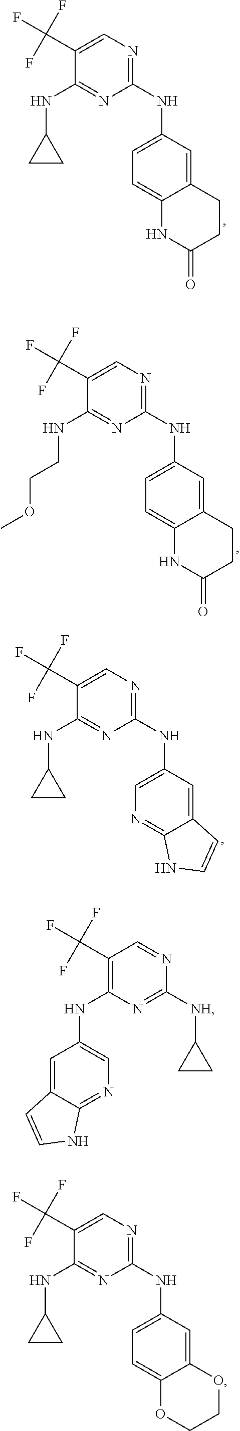

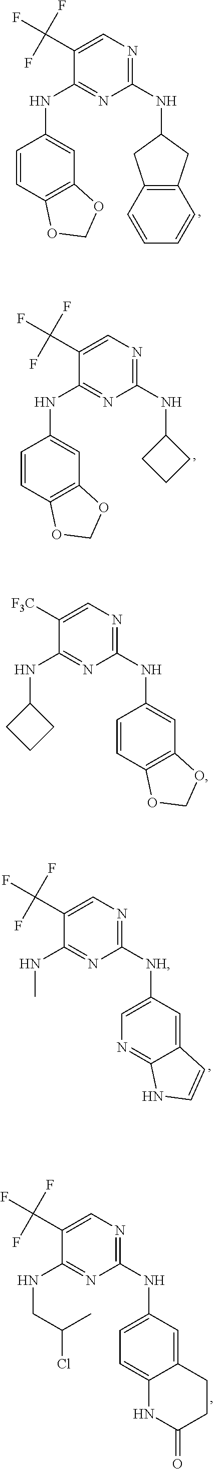 [SMILES: FC(F)(F)c1cnc(NC2Cc3ccccc3C2)nc1Nc1ccc2c(c1)OCO2 ; FC(F)(F)c1cnc(NC2CCC2)nc1Nc1ccc2c(c1)OCO2 ; FC(F)(F)c1cnc(Nc2ccc3c(c2)OCO3)nc1NC1CCC1 ; CNc1nc(Nc2cnc3[nH]ccc3c2)ncc1C(F)(F)F ; CC(Cl)CNc1nc(Nc2ccc3c(c2)CCC(=O)N3)ncc1C(F)(F)F]

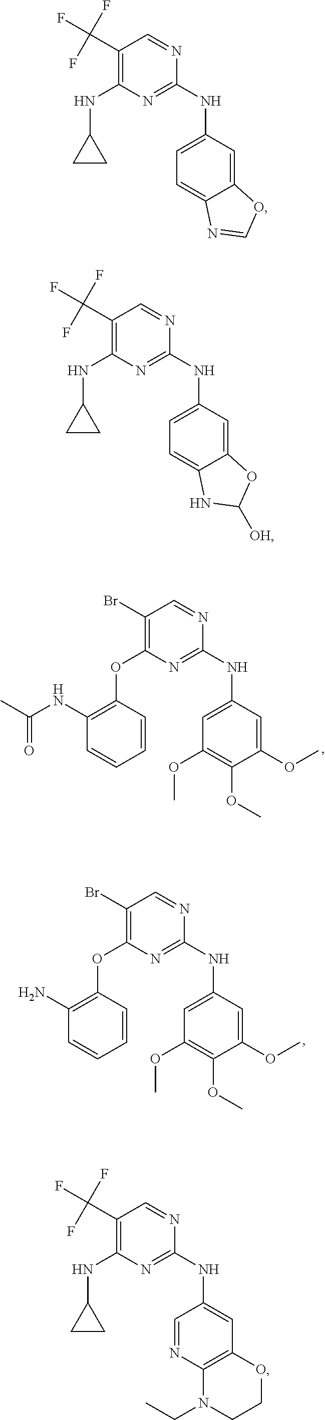 [SMILES: FC(F)(F)c1cnc(Nc2ccc3ncoc3c2)nc1NC1CC1 ; OC1Nc2ccc(Nc3ncc(C(F)(F)F)c(NC4CC4)n3)cc2O1 ; COc1cc(Nc2ncc(Br)c(Oc3ccccc3NC(C)=O)n2)cc(OC)c1OC ; COc1cc(Nc2ncc(Br)c(Oc3ccccc3N)n2)cc(OC)c1OC ; CCN1CCOc2cc(Nc3ncc(C(F)(F)F)c(NC4CC4)n3)cnc21]

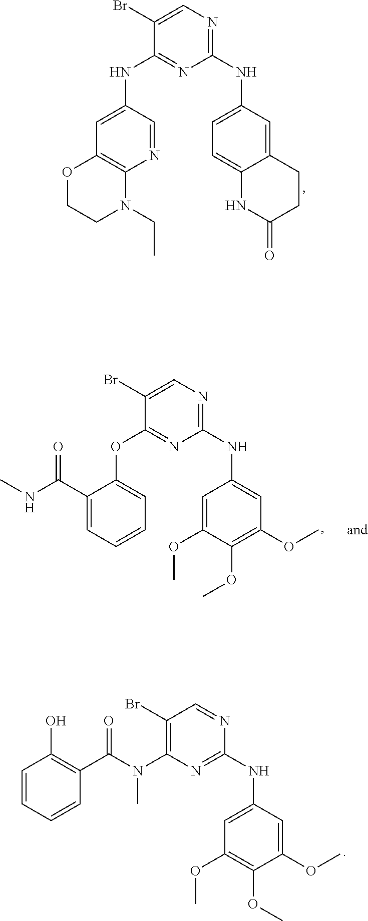

Particular examples of the presently disclosed compounds include one or more asymmetric centers; thus, these compounds exist in different stereoisomeric forms. Accordingly, compounds and compositions may be provided as individual pure enantiomers or as stereoisomeric mixtures, including racemic mixtures. In certain embodiments the compounds disclosed herein are synthesized, or are purified to be, in substantially enantiopure form, such as in a 90% enantiomeric excess, a 95% enantiomeric excess, a 97% enantiomeric excess or even in greater than a 99% enantiomeric excess, such as in enantiopure form.

Synthesis and measurements of ULK1 inhibitory activity of the compounds described herein was previously described in PCT International Application No. PCT/US2015/046777, which is hereby incorporated by reference in its entirety.

### Formulations

The compounds of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. In some embodiments, this combination therapy is sequential therapy where the patient is treated first with one drug and then the other or the two drugs are given simultaneously. In some embodiments, these are administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional agents. In some embodiments, the compounds are administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention may be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. In some embodiments, the compounds are administered alone or mixed with a pharmaceutically acceptable carrier. In some embodiments, this carrier is be a solid or liquid, and the type of carrier is generally chosen based on the type of administration being used. In some embodiments, the active agent is co-administered in the form of a tablet or capsule, liposome, as an agglomerated powder or in a liquid form. Examples of suitable solid carriers include lactose, sucrose, gelatin and agar. Capsule or tablets are easily formulated and made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Aspects of the invention include articles of manufacture, or kits, comprising the active agents described herein, and formulations thereof, as well as instructions for use. An article of manufacture, or kit, can further contain at least one additional reagent, e.g., a chemotherapeutic drug, etc. Articles of manufacture and kits typically include a label indicating the intended use of their contents. The term “label” as used herein includes any writing, or recorded material supplied on or with a kit, or which otherwise accompanies a kit.

Techniques and compositions for making dosage forms useful in the present invention are described in the following references: 7 Modem Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The compounds used in the method of the present invention may also be administered in the form of liposome delivery systems, such as small unilamellar vesicles, large unilamallar vesicles, and multilamellar vesicles. Liposomes may be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms include quantitative, qualitative or quantitative and qualitative determinations. Assessing may be relative or absolute. In some embodiments, “detecting the presence of” includes determining the amount of something present in addition to determining whether it is present or absent depending on the context.

The terms “subject,” “individual,” or “patient” are often used interchangeably herein. A “subject” is a biological entity containing expressed genetic materials. In some embodiments, the biological entity is a plant, animal, or microorganism, including, for example, bacteria, viruses, fungi, and protozoa. In some embodiments, the subject comprises tissues, cells and their progeny of a biological entity obtained in vivo or cultured in vitro. In some embodiments, the subject is a mammal. In some embodiments, the mammal is a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In some embodiments, in vitro assays encompass cell-based assays in which living or dead cells are employed. In some embodiments, in vitro assays also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. In some embodiments, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, “monotherapy” means a therapy that uses a single drug to treat a disease or condition. The single drug may be used in conjunction with various inactive ingredients, such as those used in a formulation to improve pharmaceutical properties. This is compared to the term “combination therapy,” wherein two or more therapeutic agents are administered concomitantly.

Except where otherwise specified, when the structure of a compound of this invention includes an asymmetric carbon atom, it is understood that the compound occurs as a racemate, racemic mixture, and isolated single enantiomer. All such isomeric forms of these compounds are expressly included in this invention, except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers may be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or 14C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds are generally be prepared by conventional techniques known to those skilled in the art using appropriate isotopically-labeled reagents in place of the non-labeled reagents employed.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl; n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy(phenylmethoxy) and p-trifluoromethylbenzyloxy(4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound may be independently substituted by one or more of the disclosed or claimed substituent moieties, singly or plurally. By independently substituted, it is meant that the (two or more) substituents may be the same or different.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups may be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention may be selected by one of ordinary skill in the art to provide compounds that are chemically stable and that may be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, and hexyl. Unless otherwise specified contains one to ten carbons. Alkyl groups may be unsubstituted or substituted with one or more substituents, including but not limited to halogen, alkoxy, alkylthio, trifluoromethyl, difluoromethyl, methoxy, and hydroxyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

As used herein, “heteroalkyl” includes both branched and straight-chain saturated aliphatic hydrocarbon groups having at least 1 heteroatom within the chain or branch.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “heterocycloalkyl” is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains nitrogen, it is understood that the corresponding N-oxides thereof are also encompassed by this definition.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl(4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl(phenylmethyl), p-trifluoromethylbenzyl(4-trifluoromethylphenylmethyl), 1-phenylethyl, 2-phenylethyl, 3-phenylpropyl, 2-phenylpropyl and the like.

The term “heteroaryl” as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include but are not limited to phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As used herein, “monocycle” includes any stable polycyclic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to phenyl. As used herein, “heteromonocycle” includes any monocycle containing at least one heteroatom.

As used herein, “bicycle” includes any stable polycyclic carbon ring of up to 10 atoms that is fused to a polycyclic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene. As used herein, “heterobicycle” includes any bicycle containing at least one heteroatom.

The term “phenyl” is intended to mean an aromatic six membered ring containing six carbons, and any substituted derivative thereof.

The term “benzyl” is intended to mean a methylene attached directly to a benzene ring. A benzyl group is a methyl group wherein a hydrogen is replaced with a phenyl group, and any substituted derivative thereof.

The term “pyridine” or “pyridyl” is intended to mean a heteroaryl having a six-membered ring containing 5 carbon atoms and 1 nitrogen atom, and any substituted derivative thereof.

The term “pyrimidine” or “pyrimidinyl” is intended to mean a heteroaryl having a six-membered ring containing 4 carbon atoms and 2 nitrogen atoms wherein the two nitrogen atoms are separated by one carbon atom, and any substituted derivative thereof.

The term “indole” or “indolyl” is intended to mean a heteroaryl having a five-membered ring fused to a phenyl ring with the five-membered ring containing 1 nitrogen atom directly attached to the phenyl ring.

The compounds of the present invention may be in a salt form. As used herein, a “salt” is a salt of the instant compounds which has been modified by making acid or base salts of the compounds. In the case of compounds used to treat a disease, the salt is pharmaceutically acceptable. Examples of pharmaceutically acceptable salts include, but are not limited to, mineral or organic acid salts of basic residues such as amines: alkali or organic salts of acidic residues such as phenols. The salts may be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts may be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The section headings used herein are for organizational purposes only and are not to be construed as limiting the subject matter described.

### II. EXAMPLES

The following examples are included for illustrative purposes only and are not intended to limit the scope of the invention.

### Example 1: Validation of ULK1 as a Target for Treatment of Non-Small Cell Lung Cancer

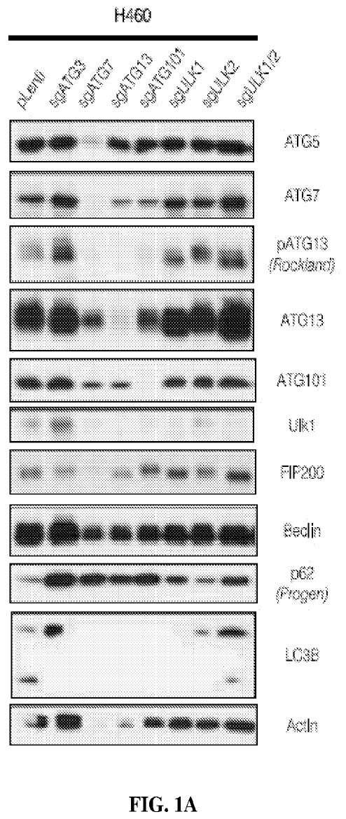

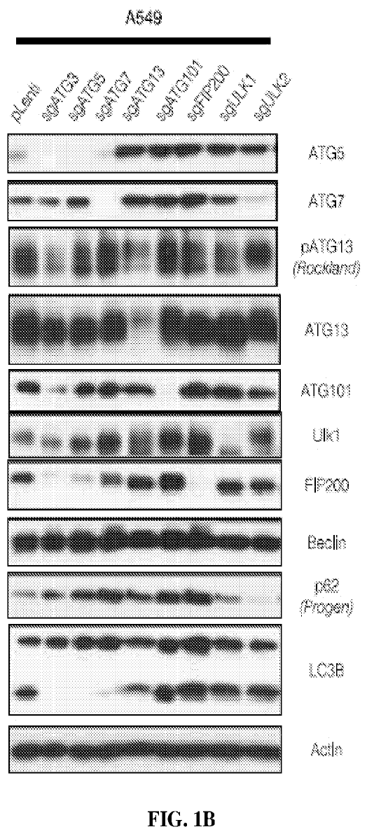

In order to assess ULK1 as a target for the treatment of non-small cell lung cancer, various genetic knockouts of NCI-H460 and A549 cell lines were prepared using sgRNA CRISPR techniques. The following genes were targeted in polyclonal pools: ATG3, ATG5, ATG13, ATG101, FIP200, ULK1, and ULK2. Lenti sgRNA was used as a control. ATG5 cell lines were generated as a positive control because ATG5 is an autophagy regulator known to impact lung cancer growth (Rao, S., Tortola, L., Perlot, T. et al. A dual role for autophagy in a murine model of lung cancer. Nat Commun 5, 3056 (2014). PMID: 24445999. https//doi.org/10.1038/ncomms4056). FIG. 1A shows Western blot analysis of isolated polyclonal pools for each sgRNA CRISPR gene knockout of the NCI-H460 cell line. FIG. 1B shows Western blot analysis of isolated polyclonal pools for each sgRNA CRISPR gene knockout of the A549 cell line.

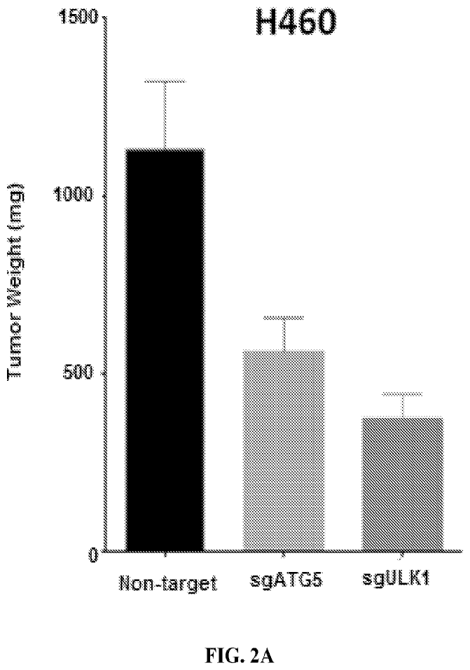

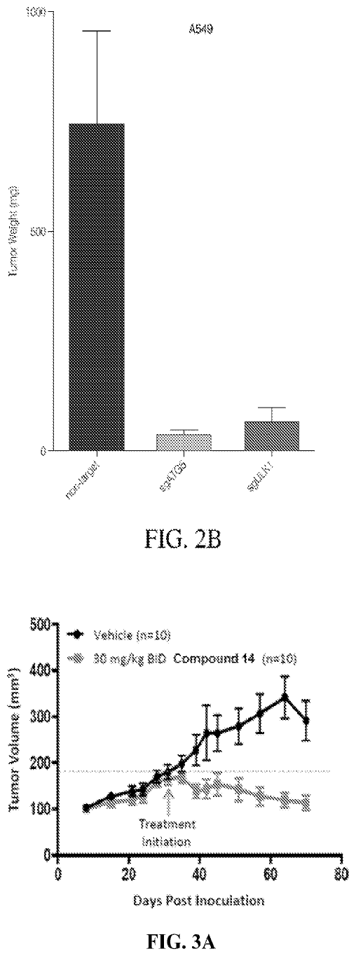

The polyclonal gene knockout cell lines prepared above were injected into the flanks of nude mice to create tumor xenografts for further analysis. Growth of the tumor cells was monitored for a number of days. The resulting tumor sizes at the end of the experiment for the sgATG5 and sgULK1 cell lines compared with control tumor cell lines are shown in FIG. 2A and FIG. 2B. FIG. 2A shows that control NCI-H460 derived tumors grew to an average size of approximately 1100 mg over the course of the experiment, while the sgATG5 knockout tumors grew to only about 600 mg in average size. Notably, the sgULK1 knockout tumors showed the most growth inhibition, only reaching an average size of approximately 400 mg over the course of the experiment. FIG. 2B shows that the control A549 cell derived tumors reached an average size of approximately 750 mg. Conversely, both the sgATG5 and sgULK1 knockout cell lines showed dramatically reduced growth. These experiments suggest that elimination or inhibition of ULK1 may provide a potential treatment for certain non-small cell lung cancers.

### Example 2: Treatment of H358 Xenografts with a ULK Inhibitor Alone

NCI-H358 cells (5M cells) were injected bilaterally into the flanks of twenty nude mice to create tumor xenografts in order to assess the efficacy of Compound 14 in treating non-small cell lung cancer tumors. After 32 days of letting tumor cells proliferate (at which point tumors had reach ˜200 mm³ in volume), one cohort of ten mice was administered 30 mg/kg twice daily doses of Compound 14, and the remaining mice administered vehicle alone. Treatment and monitoring was continued until day 72 post inoculation of tumor cells.

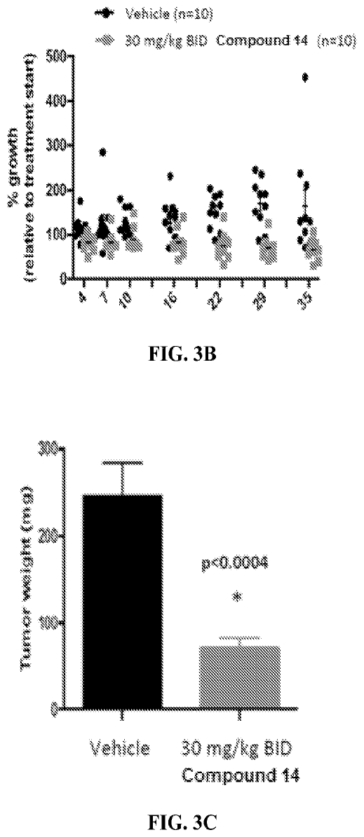

FIG. 3A shows the resulting average tumor volumes for each cohort as a function of the number of days since inoculation with the H358 cells. Post initiation of treatment, the control cohort showed substantial continued tumor growth, with tumor reaching a peak volume of over 300 mm³. Conversely, the cohort of mice receiving Compound 14 showed no growth during the monitoring period, and even a decrease in tumor size over the course of the study. FIG. 3B shows the % growth of tumor cells relative to the start of treatment for mice in the study, as well as the cohort average for the Compound 14 treated and vehicle treated mice. In agreement with the data from the previous figure, FIG. 3B shows a clear reduction in average tumor size over the treatment period for the Compound 14 treated mice, whereas the tumors continued to grow in the vehicle treated mice. FIG. 3C shows the average tumor mass at the end of the study (day 35 post treatment) for each cohort, with the Compound 14 treated mice showing substantially smaller overall tumor burden compared to control.

### Example 3: Treatment of NCI-H460 Xenografts with a ULK Inhibitor Alone

NCI-H460 cells (2.5M cells) were injected bilaterally into the flanks of twenty-four nude mice to create tumor xenografts in order to assess the efficacy of Compound 14 in treating non-small cell lung cancer tumors. After 14 days of letting tumor cells proliferate (at which point tumors had reach ˜200 mm³ in volume), one cohort of twelve mice was administered 30 mg/kg twice daily doses of Compound 14, and the remaining mice administered vehicle alone. Treatment and monitoring were continued until day 23 post inoculation of tumor cells (9 days of treatment).

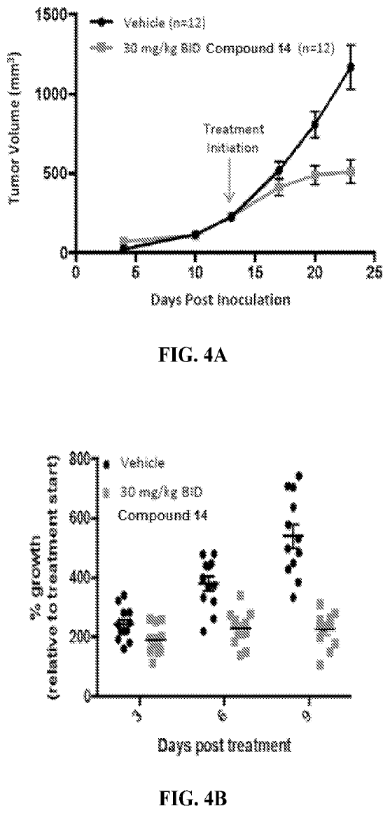

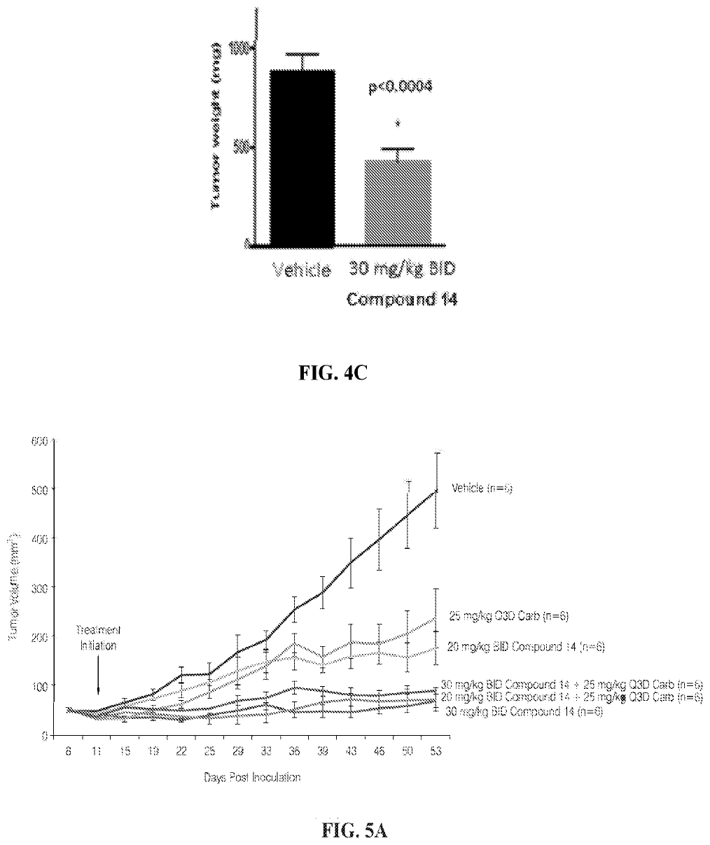

FIG. 4A shows the resulting average tumor volumes for each cohort as a function of the number of days since inoculation with the H460 cells. Post initiation of treatment, the control cohort showed substantial continued tumor growth, with tumor reaching a peak volume of over 1000 mm³. Conversely, the cohort of mice receiving Compound 14 showed substantially reduced growth during the monitoring period compared to the control. FIG. 4B shows the % growth of tumor relative to the start of treatment for each individual mouse in the study, as well as the cohort average for the Compound 14 treated and vehicle treated mice. FIG. 4B shows a clear reduction in tumor growth in the Compound 14 treated mice, whereas the tumors continued to grow in the vehicle treated mice. After 3 days of treatment with Compound 14, tumor growth had virtually stopped. FIG. 4C shows the average tumor mass at the end of the study (day 9 post treatment) for each cohort, with the Compound 14 treated mice showing ˜50% less tumor mass than vehicle treated mice.

### Example 4: Treatment of A549 Xenografts with a ULK Inhibitor Alone and in Combination with Carboplatin

A549 cells (5M cells) were injected bilaterally into the flanks of thirty-six nude mice to create tumor xenografts in order to assess the efficacy of Compound 14 in treating non-small cell lung cancer tumors both alone and in combination with carboplatin, the current standard of care therapy for non-small cell lung cancer. After 11 days of letting tumor cells proliferate (at which point tumors were ˜50 mm³ in volume), administration of carboplatin, Compound 14, or combinations began. One cohort of six mice received 20 mg/kg twice daily doses of Compound 14 alone, one cohort of six mice received 30 mg/kg twice daily doses of Compound 14 alone, one cohort of six mice received 25 mg/kg doses of carboplatin every three days, one cohort of six mice received 20 mg/kg twice daily doses of Compound 14 in conjunction with 25 mg/kg doses of carboplatin every three days, one cohort of six mice received 30 mg/kg twice daily doses of Compound 14 in conjunction with 25 mg/kg doses of carboplatin every three days, and the remaining six mice were treated with vehicle. Treatment and monitoring was continued until day 53 post inoculation of tumor cells (42 days of treatment).

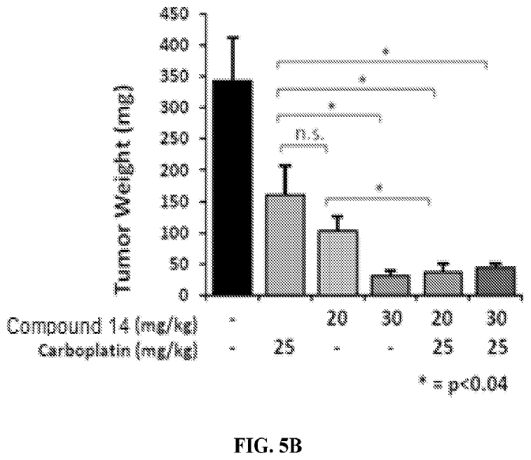

FIG. 5A shows the resulting tumor growth curves for the duration of the study. Mice in the control group receiving vehicle only showed continuous tumor growth throughout the study, reaching an average tumor volume of ˜500 mm³ by day 53. Mice administered carboplatin alone and 20 mg/kg Compound 14 alone showed similar tumor growth curves. Both showed markedly reduced growth compared to the control, but still displayed tumor growth, with final tumor average volumes of ˜200 mm³ for the carboplatin group and ˜150 mm³ for the 20 mg/kg Compound 14 cohort. The remaining groups all showed substantially diminished tumor growth, with the two combination therapy groups and the 30 mg/kg Compound 14 cohort showing tumor volumes of ˜50 mm³ at the end of the study, suggesting that these groups had successfully arrested the tumor growth. FIG. 5B shows the average tumor mass for each cohort at the end of the study, with both combination therapy groups and the 30 mg/kg Compound 14 cohort showing the smallest tumors.

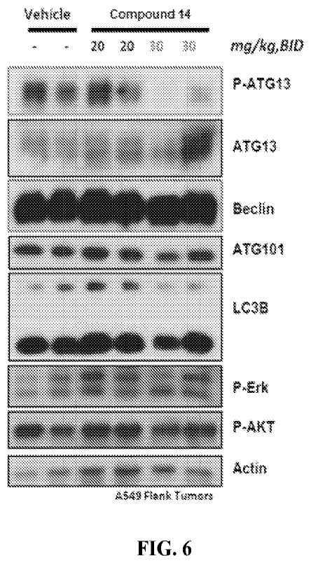

At the end of the study, Western blot analysis of the tumors from vehicle and Compound 14 monotherapy cohorts was analyzed. As can be seen in FIG. 6, the 30 mg/kg Compound 14 treated cohort showed almost total elimination of phosphorylated ATG13 (P-ATG). As ATG13 is a known substrate of ULK1, this data shows that the 30 mg/kg doses of Compound 14 were more effective at eliminating ULK1 activity in the tumors, as the 20 mg/kg cohort still showed some residual phosphorylated ATG13.

### Example 5: Treatment of A549 Xenograft Large Tumors with a ULK Inhibitor Alone

A549 cells (5M cells) were injected bilaterally into the flanks of forty-six nude mice to create tumor xenografts in order to assess the efficacy of Compound 14 in treating larger non-small cell lung cancer tumors compared to carboplatin, the current standard of care therapy for non-small cell lung cancer. After 40 days of letting tumor cells proliferate (at which point tumors were over 200 mm³ in volume), administration of carboplatin or Compound 14 was initiated. One cohort of twelve mice received 20 mg/kg twice daily doses of Compound 14, one cohort of ten mice received 30 mg/kg twice daily doses of Compound 14, one cohort of seven mice received 25 mg/kg doses of carboplatin every three days, one cohort of seven mice received 40 mg/kg once daily doses of Compound 14, and the remaining ten mice were treated with vehicle. Treatment and monitoring was continued until day 72 post inoculation of tumor cells (32 days of treatment).

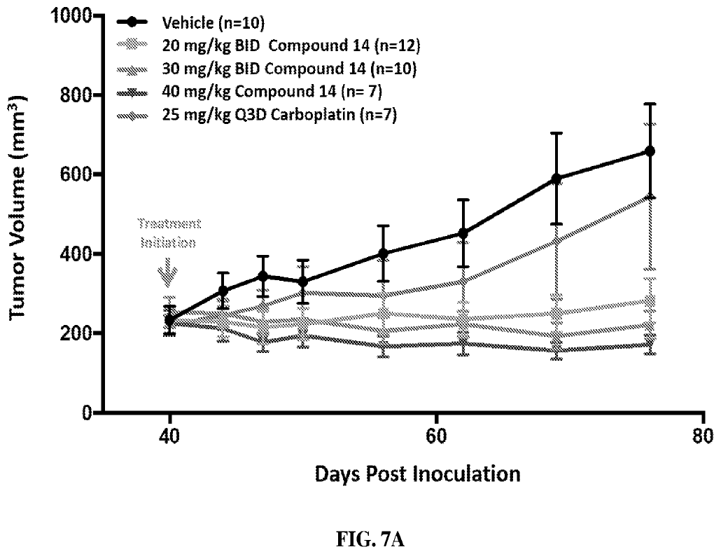

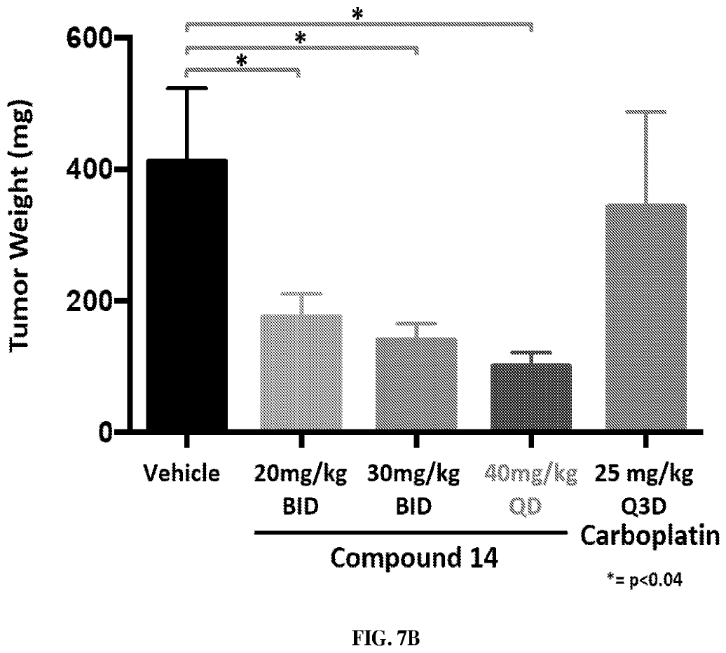

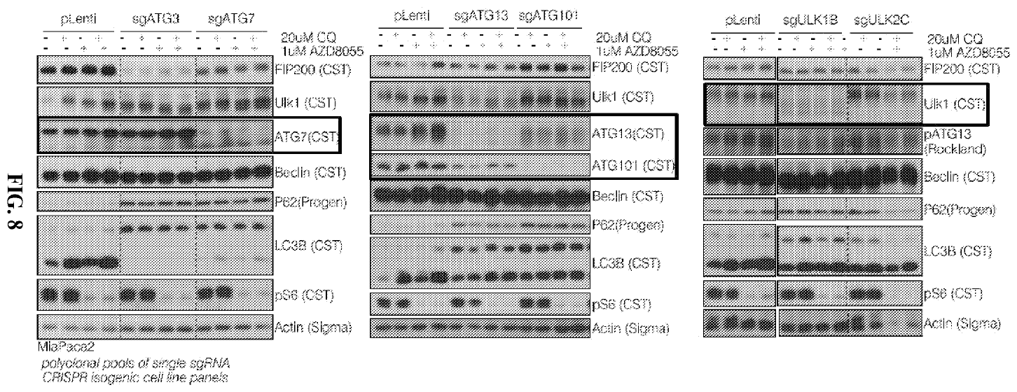

FIG. 7A shows the resulting tumor growth curves for the duration of the study. Vehicle treated mice showed continued tumor growth through the end of the study, with tumors reaching over 600 mm³ by day 72. The carboplatin treated mice showed slowed tumor growth compared to control, but still reached above 500 mm³ by the end of the study, suggesting ineffective treatment of the tumor. Conversely, each Compound 14 treated cohort showed little to no growth, or even a decrease in tumor volume compared with the start of treatment. This suggests that ULK inhibitor therapies may be more successful than carboplatin treatments in treating late-stage, large cancer tumors in patients. FIG. 7B shows the average tumor weight for each cohort at the end of the study. These results suggest a ULK inhibitor may be an effective treatment for advanced stage lung cancer.

### Example 6: Validation of ULK as a Target for Treatment of Pancreatic Cancer

In order to assess ULK1 as a target for the treatment of pancreatic cancer, various genetic knockouts of MIA PaCa-2 cell lines were prepared using sgRNA CRISPR techniques. The following genes were targeted in polyclonal pools: ATG3, ATG7, ATG13, ATG101, ULK1, and ULK2 (Mathias T Rosenfeldt, Jim O'Prey, Jennifer P Morton, Colin Nixon, Gillian MacKay, Agata Mrowinska, Amy Au, Taranjit Singh Rai, Liang Zheng, Rachel Ridgway, Peter D Adams, Kurt I Anderson, Eyal Gottlieb, Owen J Sansom, Kevin M Ryan. p53 status determines the role of autophagy in pancreatic tumour development. Nature. 2013 Dec. 12; 504(7479):296-300. doi: 10.1038/nature12865. Epub 2013 Dec. 4. PMID: 24305049 DOI: 10.1038/nature12865). Lenti sgRNA was used as a control. Each polyclonal pool was cultured in blank culture media, media containing 20 μM chloroquine (known autophagy inhibitor), media containing 1 μM AZD8055 (mTOR inhibitor), or media containing 20 μM chloroquine and 1 μM AZD8055. FIG. 8 shows Western blot analysis of isolated polyclonal pools for each sgRNA CRISPR gene knockout of the MIA PaCa-2 cell line cultured in the referenced media. Notably, the CRISPR process succeeding in attenuating ULK1 expression in the sgULK1 pool, and concomitant drop in phosphorylated ATG13 was observed in this sample.

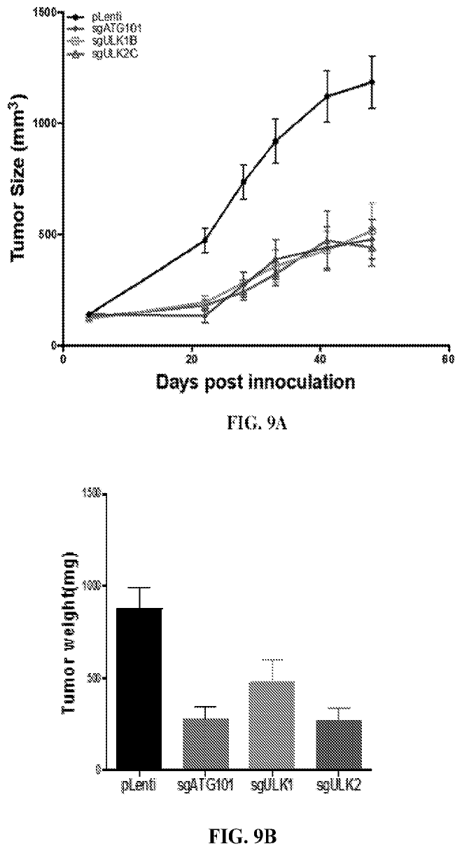

The polyclonal gene knockout cell lines prepared above were injected into the flanks of nude mice to create tumor xenografts for further analysis. Growth of the tumor cells was monitored for 49 days. The resulting tumor sizes at the end of the experiment for the sgULK1, sgULK2, and sgATG101 cell lines compared with control tumor cell lines are shown in FIG. 9A and FIG. 9B. FIG. 9A shows that control MIA PaCa-2 derived tumors grew to an average size of approximately 1200 mm³ over the course of the experiment, while the sgULK1, sgULK2, and sgATG101 knockout tumors grew to only about 500 mm³ in average size. FIG. 9B shows that the control sgULK1, sgULK2, and sgATG101 cell derived tumors reached an average size of approximately 900 mg. Conversely, both the sgULK1, sgULK2, and sgATG101 knockout cell lines showed dramatically reduced growth. These experiments suggest that elimination or inhibition of ULK1 may provide a potential treatment for certain pancreatic cancers.

### Example 7: Treatment of MIA PaCa-2 Xenografts with an ULK Inhibitor Alone

MIA PaCa-2 cells were injected bilaterally into the flanks of nude mice to create tumor xenografts in order to assess the efficacy of Compound 14 in treating pancreatic cancer tumors compared to gemcitabine, a current standard of care therapy for pancreatic cancer. After 3 days of letting tumor cells proliferate (at which point tumors were ˜100 mm³ in volume), administration of gemcitabine or Compound 14 was initiated. One cohort received 20 mg/kg twice daily doses of Compound 14, one cohort received gemcitabine, and the remaining cohort was treated with vehicle. Treatment and monitoring were continued until day 42 post inoculation of tumor cells (30 days of treatment).

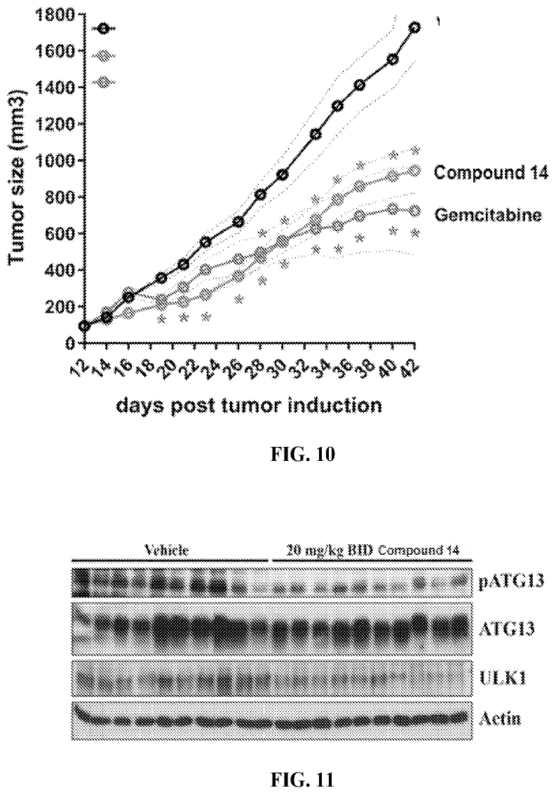

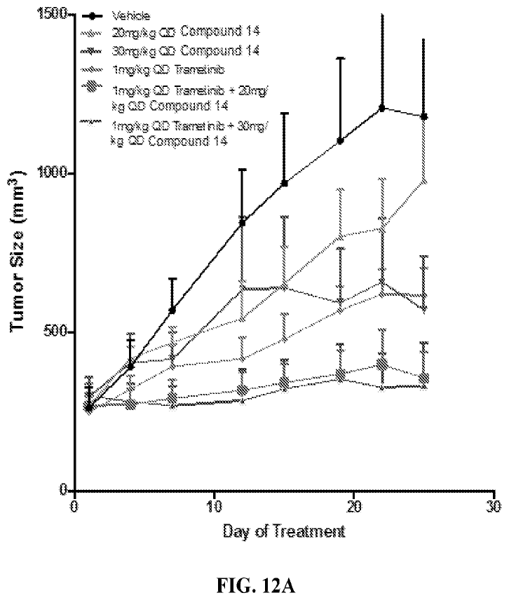

FIG. 10 shows the resulting tumor growth curves for the duration of the study. Vehicle treated mice showed continued tumor growth through the end of the study, with tumors reaching nearly 1800 mm³ by day 42. The gemcitabine and Compound 14 treated mice showed slowed tumor growth compared to control and similar efficacy to each other, with tumors reaching a final volume of ˜800 mm³ for each compound. This suggests that ULK inhibitor therapies may demonstrate similar efficacy compared to standard of care therapies for pancreatic cancer. FIG. 11 shows Western blot analysis of tumor cells at the end of the experiment. The mice that received treatment with Compound 14 showed reduced ULK1 levels compared to vehicle treated and concomitantly reduced levels of phosphorylated ATG13. Total levels of ATG13 were comparable between vehicle treated and control.

### Example 8: Treatment of MIA PaCa-2 Xenografts with a ULK Inhibitor Alone and in Combination with Trametinib

MIA PaCa-2 cells were injected bilaterally into the flanks nude mice to create tumor xenografts in order to assess the efficacy of Compound 14 in treating pancreatic cancer tumors alone and in combination with trametinib, an MEK inhibitor and current standard of care therapy for the treatment of pancreatic cancer. After letting tumor cells proliferate to the point where tumors had reach ˜300 mm³ in volume, one cohort of mice was administered 20 mg/kg daily doses of Compound 14, one cohort of mice was administered 30 mg/kg daily doses of Compound 14, one cohort of mice was administered 1 mg/kg daily doses of trametinib, one cohort of mice was administered 1 mg/kg daily doses of trametinib in combination with 20 mg/kg daily doses of Compound 14, one cohort of mice was administered 1 mg/kg daily doses of trametinib in combination with 30 mg/kg daily doses of Compound 14, and the remaining cohort of mice was administered vehicle alone. Treatment and monitoring were continued for 25 days.

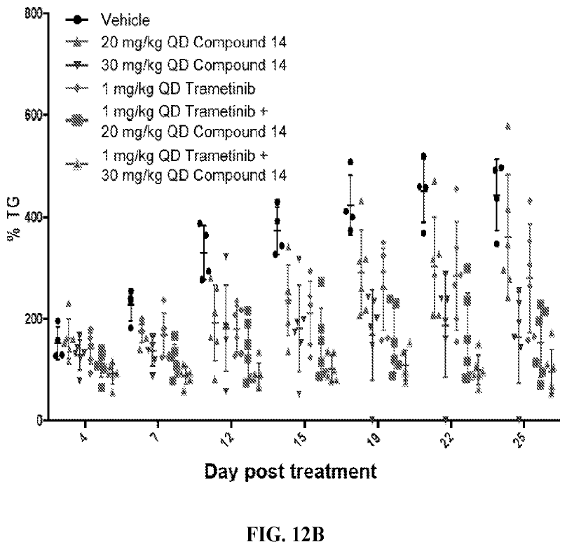

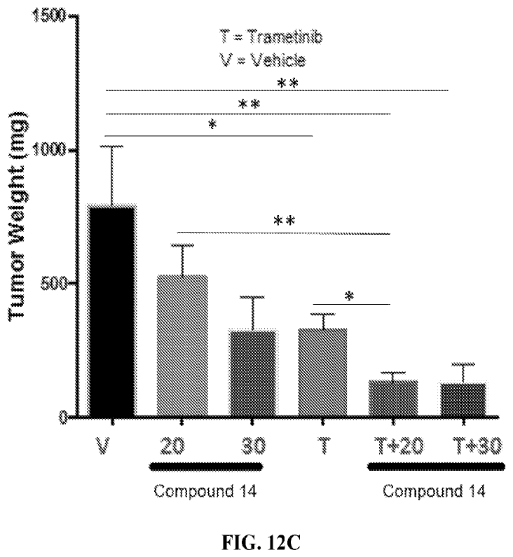

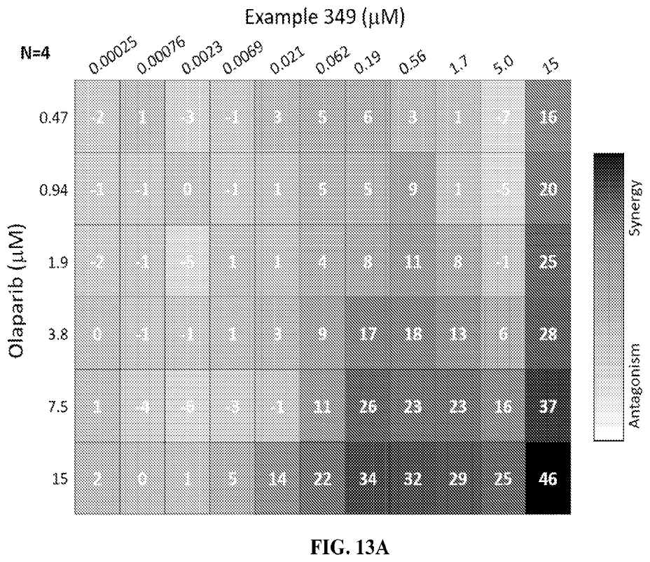

FIG. 12A shows the resulting average tumor volumes for each cohort as a function of the number of days of treatment. Post initiation of treatment, the control cohort showed substantial continued tumor growth, with tumor reaching a peak volume of over ˜1200 mm³. Each of the cohorts receiving treatment showed reduced tumor growth. The 20 mg/kg Compound 14 dosed cohort showed slightly reduced tumor growth, with the final tumor size averaging ˜1000 mm³. The 30 mg/kg Compound 14 cohort and the trametinib alone cohort showed similar tumor growth reduction, with tumors reaching only slightly above 500 mm³ at the end of the study, with substantial growth arresting after ˜day 12. Both combination therapy cohorts showed virtually no growth throughout the treatment period, with day 25 tumor sizes remaining at about −300 mm³. FIG. 12B shows the % growth of tumor cells relative to the start of treatment for mice in the study, as well as the cohort average for each cohort. FIG. 12C shows the average tumor mass at the end of the study (day 25 post treatment) for each cohort, with the both of the combination therapy treated mice showing substantially less tumor mass compared to control and each mono-therapy. This data suggests there is a synergistic effect of ULK and MEK inhibitors for the treatment of pancreatic cancer.

### Example 9: Evaluation of Syneristic Cytotoxicity of an ULK Inhibitor with Poly (ADP-Ribose) Polymerase (PARP) Inhibitors Against Triple Negative Breast Cancer (TNBC) Cells

PARPs are a family of proteins that maintain genome stability and are responsible for DNA-damage repair in cells. Since DNA damage can induce cancer cell death, PARP inhibition, which leads to the accumulation of single-stranded breaks, is a viable strategy to treat cancer. Several PARP inhibitors, including olaparib, niraparib, rucaparib, and talazoparib, have received regulatory approval for the treatment of cancer. However, the emergence of resistance has led to interest in combination therapies that can resensitize tumor cells to the effects of PARP inhibitors. Recent evidence has emerged that PARP inhibitors activate autophagy in cancer cells, and that this may lead to resistance (Ganesh Pai Bellare, Bhaskar Saha, Birija Sankar Patro. Targeting autophagy reverses de novo resistance in homologous recombination repair proficient breast cancers to PARP inhibition. Br J Cancer. 2021 Jan. 21. doi: 10.1038/s41416-020-01238-0. Online ahead of print. PMID: 33473172 DOI: 10.1038/s41416-020-01238-0 and Janice M Santiago-O'Farrill, S John Weroha, Xiaonan Hou, Ann L Oberg, Ethan P Heinzen, Matthew J Maurer, Lan Pang, Philip Rask, Ravi K Amaravadi, Sarah E Becker, Ignacio Romero, Ma Jesns Rubio, Xavier Matias-Guiu, Maria Santacana, Antonio Llombart-Cussac, Andrés Poveda, Zhen Lu, Robert C Bast Jr. Poly(adenosine diphosphate ribose) polymerase inhibitors induce autophagy-mediated drug resistance in ovarian cancer cells, xenografts, and patient-derived xenograft models. Cancer. 2020 Feb. 15; 126(4):894-907. doi: 10.1002/cncr.32600. Epub 2019 Nov. 12. PMID: 31714594 PMCID: PMC6992526 (available on 2021-02-15) DOI: 10.1002/cncr.32600). Because PARP inhibitors have been approved for the treatment of triple-negative breast cancer (TNBC), we investigated whether combining Compound 349 with PARP inhibitors would increase cytotoxicity against TNBC cells.

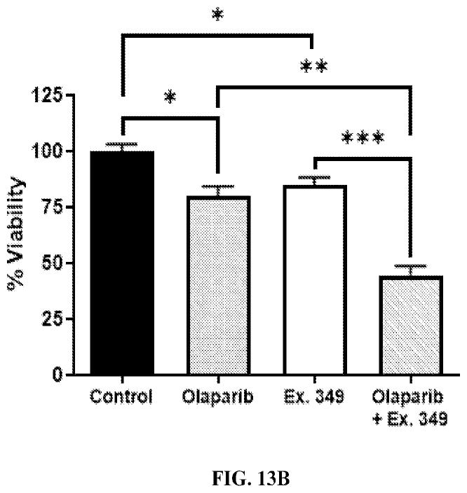

Different concentrations of Compound 349 with olaparib (FIG. 13A) were tested to measure the combined effect of inhibiting both ULK1/2 and PARP. For these experiments, MDA-MB-468 cells were treated with Compound 349 (0-15 μM) and olaparib (0-15 μM) in a dose-response matrix for 72 h, and cell viability was assessed by a CellTiter-Glo assay, and the synergistic effect was analyzed with Combenefit using the Loewe model. We observed strong antiproliferative synergy between Compound 349 and olaparib (FIG. 13A and FIG. 13B), suggesting that autophagy inhibition enhances the cytotoxic effects of PARP inhibitors toward TNBC cells. Specifically, significant synergy was observed with concentrations of Compound 349 at 0.021-15 μM and olaparib at 3.8-15 μM (FIG. 13A). The synergistic effect with olaparib demonstrated a maximum synergy score of 34. These data support the hypothesis that an ULK1/2 inhibitor in combination with a PARP inhibitor may have therapeutic utility for the treatment of TNBC.

### Example 10: Evaluation of Olaparib-Induced Upregulation of Autophaic Flux in TNBC Cells in the Absence and Presence of an ULK Inhibitor

To investigate the mechanism by which Compound 349 potentiates the cytotoxic effects of PARP inhibitors, MDA-MB-468 cells expressing mCherry-EGFP-LC3 were treated with olaparib (30 μM) and/or Compound 349 (10 μM) for 48 h and then quantified for autophagic flux. For these experiments, MDA-MB-468 cells stably expressing the mCherry-GFP-LC3 fusion protein were plated in 6-well plates at a density of 6×105 cells/well in growth media DMEM/F12+GlutaMAX-1 (Gibco) supplemented with 10% FBS (Gibco) and 1× Anti-Anti (Gibco) and allowed to grow overnight. Cells were treated, trypsinized, and then resuspended in PBS, and analytical cytometry was performed using an LSRFortessa 14-color (BD Biosciences) with a 488 nm and 610 nM laser. For flow cytometry, wild type MDA-MB-468 cells were used as the unstained control, and MDA-MB-468 cells expressing either mCherry or GFP were used for comparison. Events of 10,000 were collected for each sample.

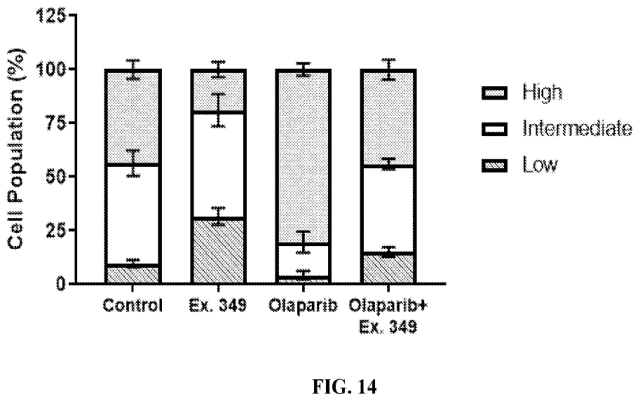

Olaparib alone was a strong inducer of autophagy, as demonstrated by an approximately 30% increase in autophagic flux compared with control cells (FIG. 14). However, concomitant treatment with Compound 349 significantly suppressed the olaparib-induced increase in autophagic flux to a level similar to that observed in untreated control cells (FIG. 14). These data suggest that Compound 349 potentiates olaparib activity, at least in part, by abrogating the ability of olaparib to induce autophagy.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating cancer in a subject in need thereof, the method comprising:
administering to the subject a therapeutically effective amount of a ULK inhibitor having a structure of Formula A: 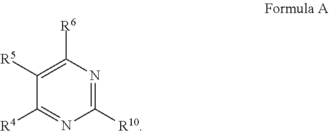
wherein in Formula A:
R¹⁰ is halogen; —OR¹¹, wherein R¹¹ is selected from the group consisting of H, optionally substituted aryl and optionally substituted heteroaryl; —NR¹R², wherein R¹ is H, —C(═O)R¹², where R¹² is C₁-C₆ alkyl or C₁-C₆ cycloalkyl, or optionally substituted alkyl and R² is H, optionally substituted aryl, optionally substituted heteroaryl, optionally substituted cycloalkyl, or optionally substituted alkyl, or NR¹R² together form a heterocycle;
R⁴ is optionally substituted amino, optionally substituted aryloxy, optionally substituted heteroaryloxy, optionally substituted alkoxy, N-heterocyclic, optionally substituted thiol, optionally substituted alkyl, hydroxyl or halogen;
or R⁴ and R¹⁰ together form a cyclic structure;
R⁵ is H, hydroxyl, optionally substituted alkyl, halo, optionally substituted alkoxy, or optionally substituted aryl, optionally substituted carboxyl, cyano, or nitro;
or R⁵ and R⁶ together form a cyclic structure; and
R⁶ is H, halogen, or haloalkyl.

2. A method for treating cancer in a subject in need thereof, the method comprising administering to the subject a therapeutically effective amount of a ULK inhibitor.

3. The method of any one of the preceding claims, wherein the ULK inhibitor is administered as a monotherapy.

4. The method of claim 1 or claim 2, wherein the subject is not simultaneously administered an mTOR inhibitor.

5. The method of any one of claims 1, 2, or 4 further comprising administering to the subject an additional therapeutic agent.

6. The method of claim 5, wherein the additional therapeutic agent is not an mTOR inhibitor.

7. The method of claim 5 or 6, wherein the additional therapeutic agent comprises carboplatin or a carboplatin analog, or an MEK inhibitor.

8. The method of claim 5, 6, or 7, wherein the additional therapeutic agent comprises trametinib, cobimetinib, binimetinib, or selumetinib.

9. The method of claim 5 or 6, wherein the additional therapeutic agent comprises erlotinib, gefitinib, osimertinib, crizotinib, pemetrexed, docetaxol, or pembroluzimab.

10. The method of claim 5 or 6, wherein the additional therapeutic agent comprises a PARP inhibitor.

11. The method of claim 5 or 6 or 10, wherein the additional therapeutic agent comprises olaparib, rucaparib, niraparib, or talazoparib.

12. The method of claim 5 or 6, wherein the additional therapeutic agent comprises FOLFIRINOX, gemcitabine, gemcitabine/abraxane, everolimus, erlotinib, or sunitinib.

13. The method of claim 5 or 6, wherein the additional therapeutic agent comprises anastrozole, exemestane, letrozole, or tamoxifen.

14. The method of any one of the preceding claims, wherein the cancer is lung cancer, breast cancer, or pancreatic cancer.

15. The method of any one of the preceding claims, wherein the cancer is non-small cell lung cancer.

16. The method of claim 14, wherein the pancreatic cancer is PDAC.

17. The method of claim 14, wherein the breast cancer is TNBC.

18. The method of any one of the preceding claims, wherein the cancer is refractory to a prior treatment.

19. The method of any one of claims 5-18, wherein the subject was treated with at least one first therapeutic agent prior to administration of the ULK inhibitor.

20. The method of any one of claims 5-19, wherein the compound and the at least one additional therapeutic agent are administered concomitantly.

21. The method of claim 20, wherein the compound and the at least one additional therapeutic agent are administered concomitantly at the start of treatment using the ULK inhibitor.

22. The method of claim 19, wherein the prior treatment is ceased prior to administration of the ULK inhibitor.

23. The method of any one of claims 5-22, wherein treatment with the additional therapeutic agent induces a cytostatic response.

24. The method of anyone of claims 5-23, wherein administering a ULK inhibitor enhances the efficacy of the additional therapeutic agent by at least 10%, at least 20%, at least 30%, at least 40%, at least 50%, at least 60%, at least 70%, at least 80%, at least 90%, or at least 95% as measured by tumor growth.

25. A method for treating refractory cancer in a subject in need thereof, the method comprising administering to the subject a ULK inhibitor.

26. The method of claim 25, wherein the ULK inhibitor has a structure of Formula A: 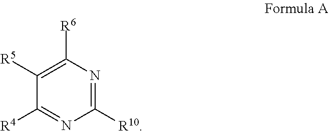
wherein in Formula A:
R¹⁰ is halogen; —OR¹¹, wherein R¹¹ is selected from the group consisting of H, optionally substituted aryl and optionally substituted heteroaryl; —NR¹R², wherein R¹ is H, —C(═O)R¹², where R¹² is C₁-C₆ alkyl or C₁-C₆ cycloalkyl, or optionally substituted alkyl and R² is H, optionally substituted aryl, optionally substituted heteroaryl, optionally substituted cycloalkyl, or optionally substituted alkyl, or NR¹R² together form a heterocycle;
R⁴ is optionally substituted amino, optionally substituted aryloxy, optionally substituted heteroaryloxy, optionally substituted alkoxy, N-heterocyclic, optionally substituted thiol, optionally substituted alkyl, hydroxyl or halogen;
or R⁴ and R¹⁰ together form a cyclic structure;
R⁵ is H, hydroxyl, optionally substituted alkyl, halo, optionally substituted alkoxy, or optionally substituted aryl, optionally substituted carboxyl, cyano, or nitro;
or R⁵ and R⁶ together form a cyclic structure; and
R⁶ is H, halogen, or haloalkyl.

27. The method of claim 25 or 26, wherein the refractory cancer is lung cancer.

28. The method of any one of claims 25-27, wherein the refractory cancer is non-small cell lung cancer.

29. The method of claim 25-28, wherein the cancer is refractory to treatment with carboplatin, a carboplatin analog, erlotinib, gefitinib, osimertinib, crizotinib, pemetrexed, docetaxol, or pembroluzimab.

30. The method of claim 29, further comprising co-administering to the subject carboplatin, a carboplatin analog, erlotinib, gefitinib, osimertinib, crizotinib, pemetrexed, docetaxol, or pembroluzimab in combination with the ULK inhibitor.

31. The method of claim 25 or 30, wherein the refractory cancer is pancreatic cancer.

32. The method of any one of claims 25, 26, or 31, wherein the cancer is refractory to an MEK inhibitor, FOLFIRINOX, gemcitabine, gemcitabine/abraxane, everolimus, erlotinib, or sunitinib.

33. The method of any one of claims 25, 26, 31, or 32, wherein the cancer is refractory to trametinib, cobimetinib, binimetinib, or selumetinib.

34. The method of claim 33, further comprising co-administering to the subject an MEK inhibitor, FOLFIRINOX, gemcitabine, gemcitabine/abraxane, everolimus, erlotinib, or sunitinib in combination with the ULK inhibitor.

35. The method of any one of claims 31-34, wherein the pancreatic cancer is PDAC.

36. The method of claim 25 or 26, wherein the refractory cancer is breast cancer.

37. The method of claim 36, wherein the breast cancer is refractory to anastrozole, exemestane, letrozole, tamoxifen, or a PARP inhibitor.

38. The method of claim 36 or 37, further comprising co-administering to the subject anastrozole, exemestane, letrozole, tamoxifen, or a PARP inhibitor.

39. The method of any one of claims 36-38, wherein the breast cancer is TNBC.

40. The method of any one of the preceding claims, wherein the cancer is characterized by abnormal autophagy.

41. The method of claim 40, wherein the abnormal autophagy has been therapeutically induced.

42. The method of any one of claims 25-41, wherein the cancer is refractory to a standard of care therapy.

43. The method of any one of claims 25-41, wherein the cancer is resistant to treatment with a standard of care therapy.

44. A method for treating a disease in a subject in need thereof, the method comprising administering to the subject a therapeutically effective amount of a ULK inhibitor.

45. The method of claim 44, wherein the ULK inhibitor has a structure of Formula A: 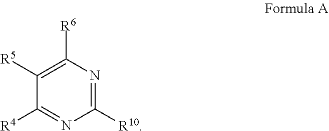
wherein in Formula A:
R¹⁰ is halogen; —OR¹¹, wherein R¹¹ is selected from the group consisting of H, optionally substituted aryl and optionally substituted heteroaryl; —NR¹R², wherein R¹ is H, —C(═O)R¹², where R¹² is C₁-C₆ alkyl or C₁-C₆ cycloalkyl, or optionally substituted alkyl and R² is H, optionally substituted aryl, optionally substituted heteroaryl, optionally substituted cycloalkyl, or optionally substituted alkyl, or NR¹R² together form a heterocycle;
R⁴ is optionally substituted amino, optionally substituted aryloxy, optionally substituted heteroaryloxy, optionally substituted alkoxy, N-heterocyclic, optionally substituted thiol, optionally substituted alkyl, hydroxyl or halogen;
or R⁴ and R¹⁰ together form a cyclic structure;
R⁵ is H, hydroxyl, optionally substituted alkyl, halo, optionally substituted alkoxy, or optionally substituted aryl, optionally substituted carboxyl, cyano, or nitro;
or R⁵ and R⁶ together form a cyclic structure; and
R⁶ is H, halogen, or haloalkyl.

46. The method of claim 44 or 45, wherein the disease is characterized by abnormal autophagy.

47. The method of any one of claims 44-46, wherein the disease is TSC.

48. The method of any one of claims 44-46, wherein the disease is LAM.

49. The method of any one of claims 44-48, wherein the subject is not administered an mTOR inhibitor.

50. The method of any of one of the preceding claims, wherein the ULK inhibitor has a structure of Formula I: 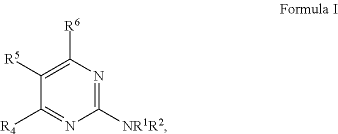
wherein in Formula I:
R¹ and R² are each individually selected from the group consisting of H, optionally substituted aryl, optionally substituted heteroaryl, optionally substituted cycloalkyl, and optionally substituted alkyl, or NR¹R² together form a heterocycle;
R⁴ is selected from the group consisting of optionally substituted amino, optionally substituted aryloxy, optionally substituted heteroaryloxy, optionally substituted alkoxy, N-heterocyclic, optionally substituted thiol, and optionally substituted alkyl;
R⁵ is selected from the group consisting of H, hydroxyl, optionally substituted alkyl, halo, optionally substituted alkoxy, and optionally substituted aryl; and
R⁶ is H.

51. The method of any one of the preceding claims, wherein the ULK inhibitor is selected from the group consisting of a 2-(substituted)amino-4-(substituted)amino-5-halo-pyrimidine, 2-(substituted)amino-4-(substituted)amino-5-(halo)alkyl-pyrimidine, 2-(substituted)amino-4-(substituted)oxo-5-halo-pyrimidine, 2-(substituted)amino-4-(substituted)oxo-5-(halo)alkyl-pyrimidine, 2-(substituted)amino-4-(substituted)thio-5-halo-pyrimidine, and 2-(substituted)amino-4-(substituted)thio-5-(halo)alkyl-pyrimidine.

52. The method of any one of the preceding claims, wherein the ULK inhibitor is selected from the group consisting of:

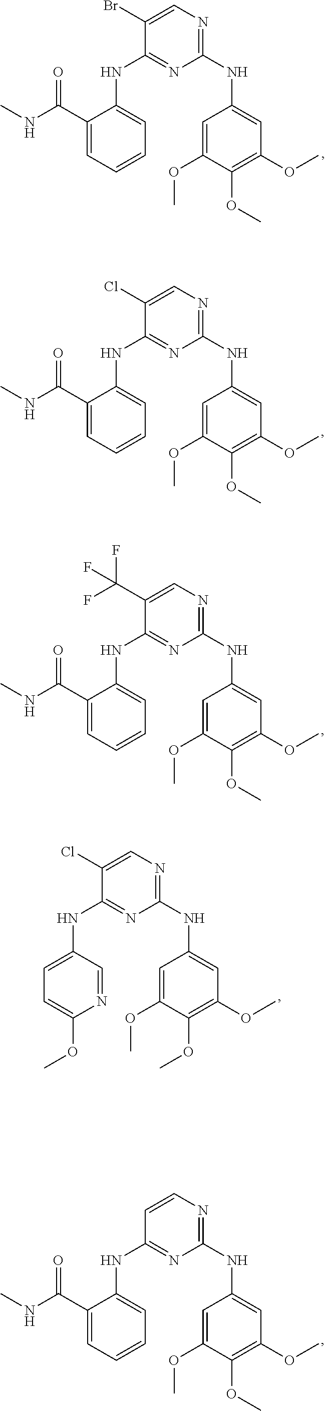

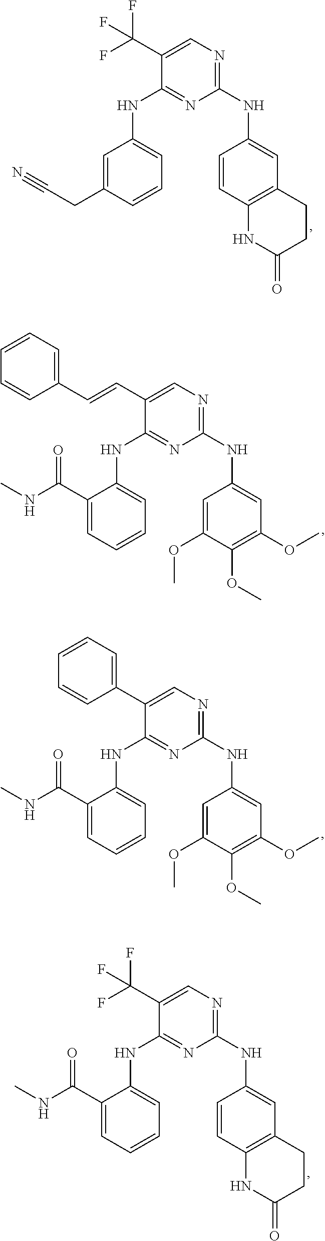

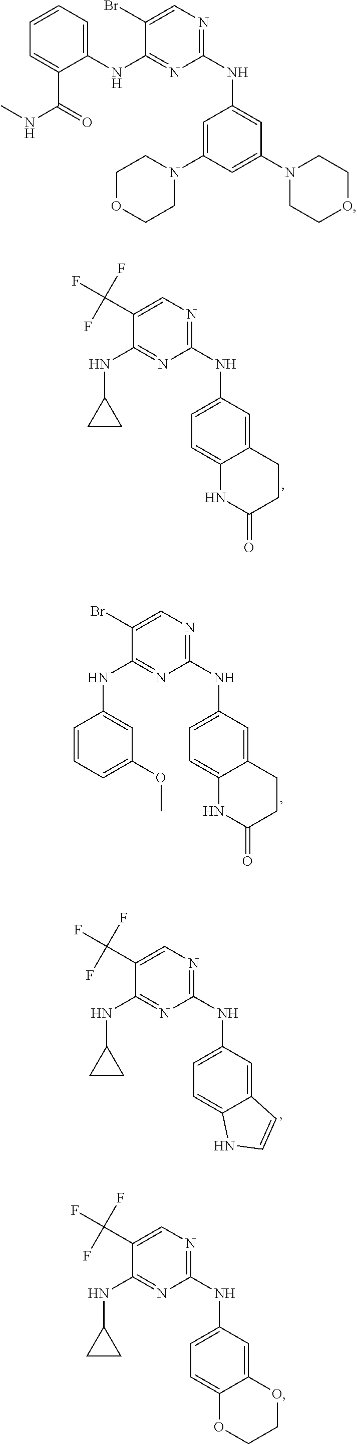 [SMILES: CNC(=O)c1ccccc1Nc1nc(Nc2cc(N3CCOCC3)cc(N3CCOCC3)c2)ncc1Br ; O=C1CCc2cc(Nc3ncc(C(F)(F)F)c(NC4CC4)n3)ccc2N1 ; COc1cccc(Nc2nc(Nc3ccc4c(c3)CCC(=O)N4)ncc2Br)c1 ; FC(F)(F)c1cnc(Nc2ccc3[nH]ccc3c2)nc1NC1CC1 ; FC(F)(F)c1cnc(Nc2ccc3c(c2)OCCO3)nc1NC1CC1]

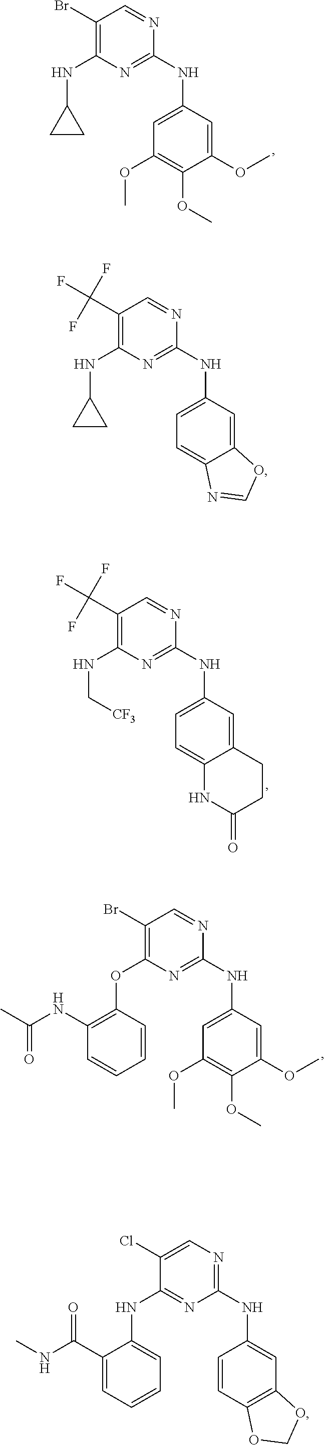

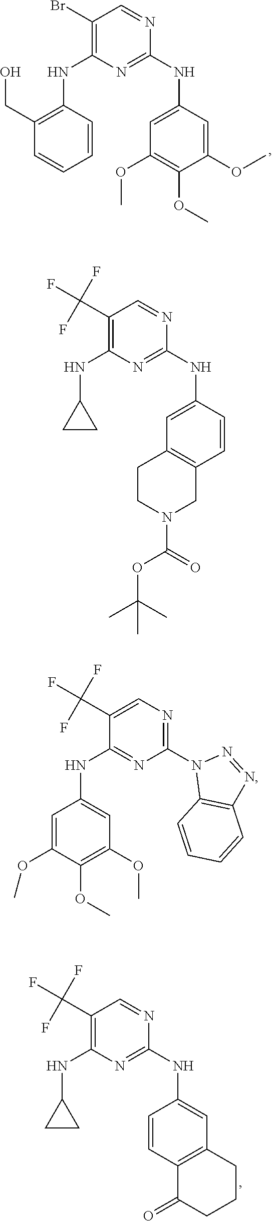

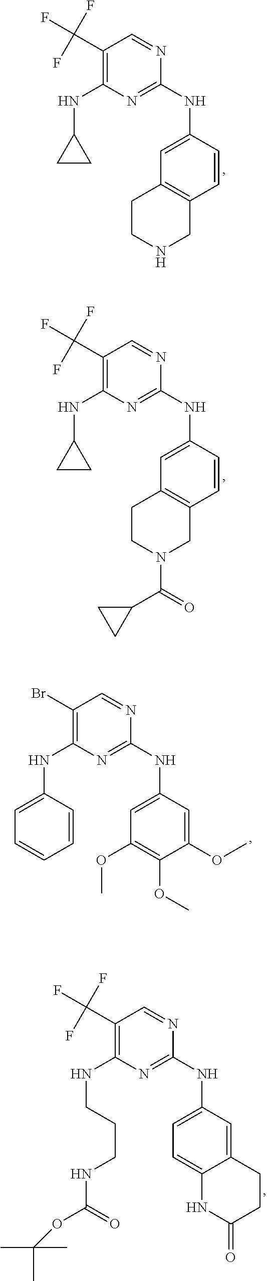

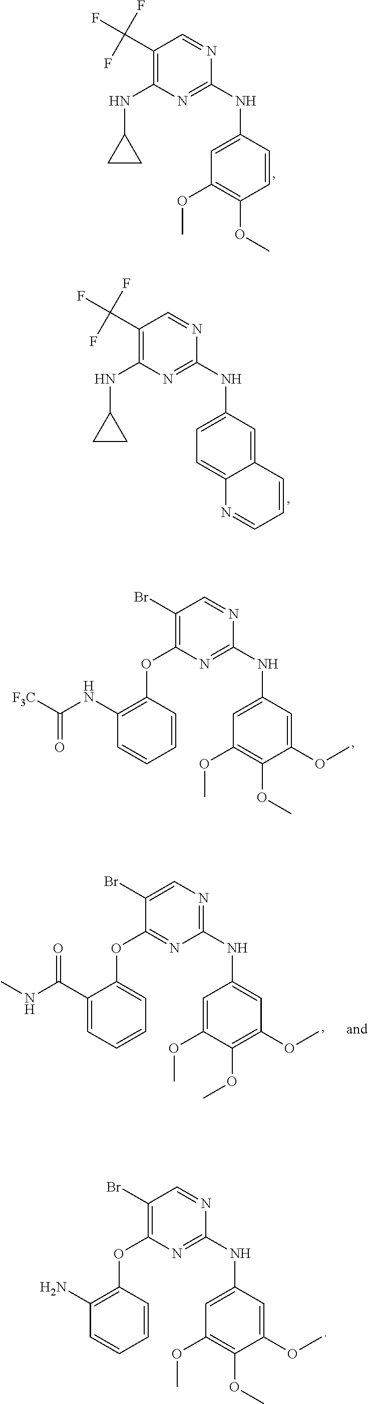

53. The method of any one of the preceding claims, wherein the method comprises inhibiting ULK1.

54. The method of any one of the preceding claims, further comprising decreasing phosphorylation of ATG13 in the subject.

55. The method of any one of the preceding claims, further comprising decreasing an amount of ATG13 in the subject.

56. The method of any one of the preceding claims, further comprising degrading ATG13 in diseased tissue of the subject.

57. The method of any one of the preceding claims, wherein the subject comprises a mutation in at least one of KRAS, PTEN, TSC1, TSC2, PIk3CA, P53, STK11 (a.k.a. LKB1), KEAP1, NRF2, EGFR, SMAD4, p16/CDKM2A, BRCA2, ALK4, or GNAS.

58. The method of any one of the preceding claims, wherein the compound is administered as a pharmaceutical composition comprising the compound and a pharmaceutically acceptable carrier.

59. Use of a ULK inhibitor having a structure of Formula A: 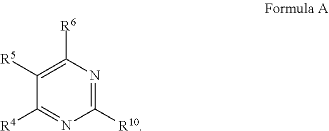
wherein in Formula A:
R¹⁰ is halogen; —OR¹¹, wherein R¹¹ is selected from the group consisting of H, optionally substituted aryl and optionally substituted heteroaryl; —NR¹R², wherein R¹ is H, —C(═O)R¹², where R¹² is C₁-C₆ alkyl or C₁-C₆ cycloalkyl, or optionally substituted alkyl and R² is H, optionally substituted aryl, optionally substituted heteroaryl, optionally substituted cycloalkyl, or optionally substituted alkyl, or NR¹R² together form a heterocycle;
R⁴ is optionally substituted amino, optionally substituted aryloxy, optionally substituted heteroaryloxy, optionally substituted alkoxy, N-heterocyclic, optionally substituted thiol, optionally substituted alkyl, hydroxyl or halogen;
or R⁴ and R¹⁰ together form a cyclic structure;
R⁵ is H, hydroxyl, optionally substituted alkyl, halo, optionally substituted alkoxy, or optionally substituted aryl, optionally substituted carboxyl, cyano, or nitro;
or R⁵ and R⁶ together form a cyclic structure; and
R⁶ is H, halogen, or haloalkyl,
in the preparation of a medicament for the treatment of cancer or refractory cancer.

60. Use of a ULK inhibitor having a structure of Formula I: 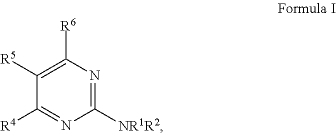
wherein in Formula I:
R¹ and R² are each individually selected from the group consisting of H, optionally substituted aryl, optionally substituted heteroaryl, optionally substituted cycloalkyl, and optionally substituted alkyl, or NR¹R² together form a heterocycle;
R⁴ is selected from the group consisting of optionally substituted amino, optionally substituted aryloxy, optionally substituted heteroaryloxy, optionally substituted alkoxy, N-heterocyclic, optionally substituted thiol, and optionally substituted alkyl;
R⁵ is selected from the group consisting of H, hydroxyl, optionally substituted alkyl, halo, optionally substituted alkoxy, and optionally substituted aryl; and
R⁶ is H,
in the preparation of a medicament for the treatment of cancer or refractory cancer.

61. A ULK inhibitor having a structure of Formula A: 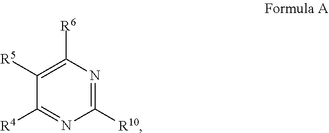
wherein in Formula A:
R¹⁰ is halogen; —OR¹¹, wherein R¹¹ is selected from the group consisting of H, optionally substituted aryl and optionally substituted heteroaryl; —NR¹R², wherein R¹ is H, —C(═O)R¹², where R¹² is C₁-C₆ alkyl or C₁-C₆ cycloalkyl, or optionally substituted alkyl and R² is H, optionally substituted aryl, optionally substituted heteroaryl, optionally substituted cycloalkyl, or optionally substituted alkyl, or NR¹R² together form a heterocycle;
R⁴ is optionally substituted amino, optionally substituted aryloxy, optionally substituted heteroaryloxy, optionally substituted alkoxy, N-heterocyclic, optionally substituted thiol, optionally substituted alkyl, hydroxyl or halogen;
or R⁴ and R¹⁰ together form a cyclic structure;
R⁵ is H, hydroxyl, optionally substituted alkyl, halo, optionally substituted alkoxy, or optionally substituted aryl, optionally substituted carboxyl, cyano, or nitro;
or R⁵ and R⁶ together form a cyclic structure; and
R⁶ is H, halogen, or haloalkyl,
for use in the treatment of cancer or refractory cancer.

62. A ULK inhibitor having a structure of Formula I: 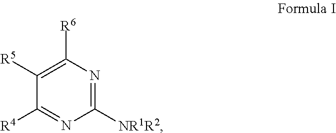
wherein in Formula I:
R¹ and R² are each individually selected from the group consisting of H, optionally substituted aryl, optionally substituted heteroaryl, optionally substituted cycloalkyl, and optionally substituted alkyl, or NR¹R² together form a heterocycle;
R⁴ is selected from the group consisting of optionally substituted amino, optionally substituted aryloxy, optionally substituted heteroaryloxy, optionally substituted alkoxy, N-heterocyclic, optionally substituted thiol, and optionally substituted alkyl;
R⁵ is selected from the group consisting of H, hydroxyl, optionally substituted alkyl, halo, optionally substituted alkoxy, and optionally substituted aryl; and
R⁶ is H,
for use in the treatment of cancer or refractory cancer.
